SEQUENCE LISTING (10 sequences)

SEQ ID NO: 1 - DNA, 78056 bp, Bos taurus
taggaataatcaatattgtgaaatgaccatataccaaatgcaacctacagattcaatgagatctccatctaacttccaatagcatttttcacagaagtagaacaaaaaatttcacaattcatatggaaacacaaaaggccctgaatagccaatgcagtcctgagaaagaagaatggagttggaggattcaaccatcctgactttagattatactacaaagctacagtcatcaagacagtatggtattggcataaaaacagaaatatagacaaatggaacaagacagaaagcccagaaataagcccatgaacctatgggtaccttattcctgacaaaggaagcaagaatatacaatggggcagacagcctcttcaataaatggtgctgggaaaactggacagctacatgtaaaagaatgaaattagaacacttcctaacaccaacagttcagttcagttcagctggtcagtcgtatcgactctttgcaaccccatggactgcagcatgccaggcttcccttgtccatcaccaactcctagagcttactcaaactcatgtccattgagttggtgatgccatccaaccatctcatcctctgtcgtccccttctcctcccaccttcaatcattctcagcatcagggttttttccaatgaggcagttctttgcatcaggtggccaaagtattggactttcagcttcagcattagtccttccgatgaatattcaggactgatttcctttaggatggactggtttgatcttgcagtccaaatgactctcaagagtgttctccaacaccacagttcaaaagcatcaattcttcagcactcagctttctttatagtccaactctcacaaccatacatgactactggaaaaaccatagctttgactagatggagctttgttggcaaagtaatgtctctgctttttaatatgctgtctaggttggtcataacttttcttccaaggagcaagcatctttaatttcatggctgcagtcaccatatgcagtgattttggagcccccaaaataaagtctgtcactgtttccactgtttccccatctatttgccatgaagtgatgggaacagatgccatgatcttagtttcctgaatgttgagttttaagtcaactttttcactctcctctttcactttcatcaaaaggctctttaggtcttcttctcttaaccataaggatggtgtcatctgcatatctgaggttattgatatttctcctggcaaacttaattccagcttgtgctttatccagtccagcatttctcgtgatgtactctgcatataaattaaataagcagggtgacaatatacagcctcaatgtactcctttcctgatttggaaccagtatgttgttccatgtctagttctaactgttgcttcctaacttgcatacagatttctcaggaggcaggtcaggcgttctggtattcccatctctttaagaatttcccacagtttgttgtgatccacacagtcaaaggctttggcacagtcaataaagcaaaaatggatgtttttctggaacgctcttactttttcgatgatccaatggatgttggcaatttgatctctgattcctgtgccttttctaaatccagcttgaacatctggaagttcatggttcatgtacttttgaagtctggcttggagaatttgagcattactttgctagtgtgtgagatgagtgtaatcatgcagtagtttgagcattctttggcattgcctttctttgggattggaatgaaaactgaccttttccagtcctgtggccactgctgagttttctaaatttgctgccatattgagtgcatcactttcacagcatcatcttttaggatgtgaaatagctcaactggaattccatcacctcccctagctttgttcataatgatgcttcctaaggcccacttgacttcacattctaggatgtctggttctaggtgagtgatcacaccatcatggttatctgggtcatgaagttctttcttgtagagttcttctgtgtattcttgccacctcttcttaatatcttctgcttttgttaggtccataccatttctgtcctttattgtgcccatctttgcatgaaatgttcccttggtatctgtaattttcttgaagagatctctagttcttcccattctattgtctccagggtgaccacccccagaccctgtacctggggtcatataaccacagtttccacagtgacatcccttccaaaccctgaacttggggttgcacgtccatgttctccagcgtgacacaccctcttggaccatgcactggggtcacatgtccaggttccaggctaacacccccccccatactctatacctggcatcaacgtcctcatacagcagggtgaccgaaccctcaacatcatgtacctggtgttgaaactccacagtttctgcacctcatcagaccttgtacctggagtcacatgtccacagtctctagggtgacagcacttcaccagacctttgaccagtgttcacacatccagtctccagggtgatgcccacttccagaccctgtacctggggtacatgttcacagtctccagggtgacagccccccagactctgtagctgggttaacagacccacctttccagggtgactacaccactccatattgtgcacctggggtcacacatccacaatctctggggtgacctctcccagaccctgtacgtgggtcacacatccacagtcccagggtgaccccacttcccagactgtggaactgggttcacatgtccacagtttccaggtgaatccccctccccaaagcctgtacctggggtcacacatccacagtctccagagtgaccctagcctccagaccctctccctggggtcacatgtccatggtctacagggagatacccctcccagaacctgcacctggggtcacatggccacagtctccagggtgaacccctaccagaccctgtacctggggtcacatgttcagagtctccagggttacctgcctcccagaccctgcaccttggttcacatgtctgcagtctccagtgtgaccccactcctgtacctgtggtcacatgtgcagattccagggtgacacccctcccagaccctgcacctggggtcacacgtatgccatctccagggtgaccccgcctcaccagaccttttacctggggtcacaccttcacagtctccagggtaacccccccacccagactctgcacttggggtacaaatccacagtttccagggtgacaccccctcagaccttctacctgaattcagagtttgatagcctcctgggagaccccaccacaccagcgagtgcacctggcttcacacgtccacagcgtccaggatgacatgcccccagaccctgtacctagggtcacatatctctagttccctggtgacccctccaagaccctgaacctggggtcatatgtctgcagtctccagggtgaccaaccacagacactctacctggggtaatatattcacagtctacagggtgacaacccactcagaccctgaacctggggacacatgtccacggtctccagggtgatcacacactccagaccctgtacctggggtcacatatccacagtctccagggaaacccaactgcccatactgtgcacctgggggtcacacatccagtctccagggtgaccccccgccccatatcctgtaccttgggccacatgacctcagcctccagggtgaccccaccctcaacatcatttacctggggccaaatctccacactctccagggtgacctcctcccagaccctgcacgtggggtcacatgtccacagtctccagggtgaccccatgtcacagatcctgcacctgagtcacatgtcaaccgtctccatggtgacccctcccagactgcacctggggtcacatatccatagttcccatggtgatcccaccctggccctgtacctgtggtcacatgtccacagtttcagggtgagttccctcccacgttctgtacctgaggtcacatgtccatagtctccagggtgaccccatcttctagacattgtaccagggttcacagatccacagtctccagggtgatctccctctccataccctgtacctggggtcaacatcctcagcttccaggatgacccaatcctcaacatcgtgtaccgtgggtcaaacgtccacagtctccagggtcactgcacctcactagaccttgtacctggggtcacatgtgcacagtctctagggtgacattacctcaacataccttttaaatgggttcacacgtccacagtctcccagggtgactcccctctcagcctcctgcacctgagatacacattcacagtctccagggtgacatcccccccagacactgtacctgggttcacaggtccacctcctccagggtgacctcaccacaccagaccacgcacctgtgatcacacatccacagtgtccagggtgacaccctcccagatcctgtacctaaggtcacatatctacagttccctgagagaccctcccaaaccctgtacctgggtcacacatccacagtcccagggtgaccccacttcccagactgtgaaaccgggttcacatatcaatagtttccaggtgaattcccctccccaaaccctgtacctagggtcacacgtccacagtcccagggtgaccctagcctctacctggggtcacatgtccacattctacagggtgacccccctcccaggccctgctcctagggtcatatggccagtttccacagtaaacccttcccagaccctgtacctggggtcacatgtccagagtctccagggtgatccacatcccaaactcttcacctggcatcacacgtccatagtctcaagggtgacaccctcccagactctgaacctggggtcacatgtccacagtctccagggtgacccccacccgaccctgcccctggggtcacacgtcttcagtctccagggtgacacccctccccagacactgtaactagagccacatgtccacagtctacaagggtgaaccccgccccccccccataatctgcatgtgggttcacatatccacagtctccttggtaaccttgcttcccagtaacggcacctggattgcacatccacagtcttcatggtgaccccctcccagactctgcacctgagttcaaatgtctacagtctccagggtgacccctcccaaaccctgcagagggcctcacatgtccacagtctccaggctgaacccccctcccagaatctatacctggagtcacatgtccacagtctccagggtgacaccccctcccccagaccttgggggtcacattgaaacagtctccaggtgaccatcttcccagaccctgcacctagggtcacaagcccacatctccaatgtgacccctctcctgctccaggggtcacatgtccacaaattccaaggtgacacctctcccagacactgcacctgggttcacatgtcccactgtctccagggtgacaacccccatactctgtacctgggttcacaggtccacagtctctagggtgactctgccacatcagactgcacagctgtgggcacatgtccacagtttccagggtggcaccctcccagatcctgtacccagggtcacatatttacagtcccctgggttaacactcccagaccctgtacctgaggtcatttgtccacgttgtccagggtcaacccttcccagaccctacacctcggctcacatgtccacagtctccagggtgacctcctcccaaccctgaacttggagtcacatgtccagtctctagggtgatcacccccataccctgtacctgggatcacaaaactacagtctccagggtgaccctgtgcccagacactgaatctggggtcacacatccacagtctccagtgtgatgcccactcccagaccatgcaccaggggtcacacatccacagtctccagggtgacagcctctgagaccctgaacctggggtcacatgcccacagtctacagggtgacgacccctcccagaccacatacatgggttcacaggttcacagtctccaggataacaccctcccagaccctgtacctatggacacctatctacaatccccttggtgtcccctcccagacactatacctgggttcacatgtccacagtcttcagggtgacaccctccagaccctgtacctagggacacctatctacagtccccttggtgtcccctcccagacactatatctggattcaaatgtccacagtctccaggttgaccccaccctcaagaacctctacctgtagtcatatgaccacagtctccagtgtaaacccacctcccatatcctgcacctggagtacatgtccagtctccagggtgaccccacagtccagaccctgtacatagggtcacagacctcagcctcaagagtgatactctcccagagtttttacctggggccacatgttcatagtctccagggtgatcctctctcaactctgcacctggggtcacacatacaaagtctccaggtaacaccccccaataccctgtgcctggtgttgcacatccacagatttcacagtgaccccacctcccggaccctgcatctgaagtcaaatgcccacagtctccagtgtaaactcacatcccatgctgttcctggggtaaatgtccacattatccaggatgaccccacctcccagaccctgttccttgggtctcacttacacagtctccagagtgtccccacctctcagaccctccatctgggatcgaacattgccagtatccagggtgaccccctcccagactctacacttggggtcacatgtccagagtttccagggtgactgcctcccgaacctgtagctgtggtcacaggtccacagtcttcagggtgacacccctctccagacaatgtatctggagtcacacattcacagtctgctaggcgaacccagcccccaaaccctgcacatgggaccccatgttcagtggggagtgttgactaccagcctacaacaaccagctggctgttgctgtcagcagcctaattgggtgaaaaatggactgggtgatgacttcccaataagaagtggcaagtagcatgttcctttcagaaaactcaaataatgaaccaaagttgttgtcataatgtacagacaaatgacctggtgtgtttcttcatggttggttgagtctgcaactatccacctttcccaggatgatcatatagattttgccatattactttgattccagcctcaacataacatgtttccctgtacatttagagctgggtaaagacactcctggagaaggcaatggcaccccactccagtactcttgcctggaaaatcccatggatggaggagcctggtaggctgtagtccatggggtcacgaagagtcagacacaattgagtgactttactttcacttttcactttcatgcattggagaaggaaatggcaacccactccagtgttcttgcctggagagtcccagggacgggggagcctggtgagctgccatctatggggtcgcagagtcggacacgactgaagcaacttagcagcagccgcagcaaagacactcctagtgtacaaacactgtacagtttgaggagtatagacagcagtggagagtgctctatgaatgtggatggccaggtctgtttttaccctgagtaggtgaaacgtactgtcaggtgacctcacagcaagaagtggcaagctgcctgtcatgagaagtgaagttgcccactcatatgcgactctttgcaatcccatggactgtagcctatggaggtcttctgtccatggaattttccagtcaagagtactaaagtgggttgccaattctttttccagggaatcttcgcgacctgagtatcaaacctgggtctcccacatcgcaggcagacactaccctcttagctaccacggaagcccaccagattcattgaaaactcaactgtttacccgaagttgttctgggattgaagagtagaatgagcctgctgtgtttttctgccaggtgggatgctgtaactacacaggtttcacagggggaaatgactgctttggaacgtccgatcccaattccaggttcacaaaaagttgtctccctgcagttttaaagctgtgtaaatgccttcctaggacacaatgggctctctgtgaatgttgacttctgtatgtgttcttaccctattcagcgaaaacagtactgtcagaggacttcccatcaagaagtggcaagcagagtgtttctctcaggtaacagaagtacttgatgagacttgcagtcctaaagaagggaaaaacgacccgtgtgcttttccgtcaggctggagattgcaagtattccaggtttgctaggtgaaaatactgctttctcttcaggccccgagttctacgttcagagaaacatttccacacaggttcagagctgtgccaagattccaagaacacactgtacaatttctcagaaaagacttcagtgaagaatgtcctgcaagtggttctgccataattgttaccctgagtgggtggaattacactgtcagatgactttttagcattaaaaagcaagtgcctatgctttcagaaaaggcagatactaaagctgcgctcgtaagaaacaggcaaatgaacccgtgtgcatttctttcaggcttaagactgcaaccaccctgcagttacacgaaaaagtgctgcttctgccattaggctccaaatcccatgtgaacagaaataaatccccctgtcattagaaagctgtgtaagtgagcccaggccagaatgcaccataccatgagtaaagaggtcagtggggagtgttctgagagttcaatagtgagtgttctggaagtgttgactgccatacctgtagtcagcagcctaatttggtgaaactggactgggtgatgactttccagtaagaaggagcaagctgcaattacctttcagaagactcataatggcccaaagctgttgtaataatgcccagacaaatgagtcactgcgtttcttcctagctggttggtggctgcaactatccaggtttaaggggaggaatatactgactttgccatattaccttatttgcagcttcaggtaacatatttccctgcacatttagagctgtgtaaagacactcctagagaacaaactgtaccatttgtggagcaaagatagcggtggagagtgctctgtgaatgtggatggtcaggcctgtttttagcctgatgaggtgagatgtaatatcaggtgacctcacagccagagtggcaagccacctgattcatgagaagtgaagttgctcagttgtatgtgactctttgcactcccatgcaatgtagcttaccaggctcctttttcgatggaattttccaggcaagaagtactggtatggcttgccacttccttttccaggagatattcctgacccagggatcacacccgggtcttcaatgttgcaggcagatgctttaccctctgggtcaccagaatccggctggattcattgaaaattcactgatttaacaaaagctgtcctggaatggaagagtagaatgagcctgttgtatttctctggcaggtggtatgctgtaactacaaggcttcacaaggacgaatgctttgggacatcagtccccaattccacgtgcacaaaatgacatctacctatagtttcaaagctgtgtaaatccattcctaggacacaatgggctatccgtgaaggttgacttttgcatctgtttttacactgatcagctaaaactgtactgtcagtggacttcccatcaggaagtggtaagcagaatgtttctctcagaaaacacaagtccttgatgagacttgcggccctaaagaagggaaaatgcccccgtgtgttttctgtcagtctggagactgcaattattccaggttccttaggcacagattttgctttcatttcaggccctgagttctaggttcagagaaacatttccccacagcttcagagctgtgcaaaacactcctagaacacactgtatcattcccttagacaagagaccagggaagagcattctgtgagcgcttctgcgatatcagttgttaccctgagtgggaagaattacactgtcaggtgacttggtagcattaataagctagagtctgtgctttcagaaaaagcagacacttttagcaaagttgccttcgtaaggaagtggaaaatgaacccgtgtgcctttctttcaggcttaagactgcaatcaccctgtggtcaaaaaagaaatactgcttctgacattaggctaaaaagcccacatgaaccaaacaaatgcccctgtcgttagaaagctgtgtaagagacccaaggacagactgcaccagtccatgagtaaagagttctggggggagtgttctctgggtgttgactgccatacctgtagtcagcagcctaattcggtgaaactggactgggtgatgacttcccagtaagaagtggcaagctacatgttccttttggaagactcaaagaatggccaaaagctgttgtcataatgcccagacaaacgagccagtgcatttcttgctggttgaaggctgcaactatccagctttcagcagaggaatattctgattttgccatattacctggatttgcagcttcaagataacacatttccctgcacaagtagaggtgtgtaaagacactcctagtgtacaaactctgtaccacctggggaacaaagatagcgtggacagtgctctgtgaatgtggatggccaggcctgttttcaccctgaagaggtgaaaagtactgtcgggtgaccttgcagccagaagcggcaagacgcctgcttcatgagaagtaaagttgttcagtcattgcgactctttacaatcccgtggactgtagcttaccaggctcctccatccatggaattttccaagcaagactactggagtggcttgccatttaattctcaatggatcttcctgatccaaggatcaaacccaggtcacccatgttgcaggcagatgctttaccctttgagccaccagggaagcctgccagattcattgaaaattcaactacttaactaaagctgttctggaatggaacagcagaatgagcctgttgtgtttttctggcaggtgagctgctgcaaccacacagtttcccaaggggaaatgactgttttgggatgtcagaccccatttccaggtgcacaacaggacgtctccctgaagattcaaagctgtgtaaacgcattcctaggatacaatgtgctctctgtgaatgttgacttttgtatctgtttttctactgattagctaaaactgtactgccaagggacttcgcatcaaaaagtggcaagcagacccatggtcctaaagaagagaaaaatgaccccatgaacttttccatcaggctggagattgtaagtattccaagttcattagtaacaaatgctgctttcatttcaggccaccatttctaggttcagagaaacatttccccgcagattcagagctgtgcaaagacactcctagaacacactgtatcattccctaagaaaagagttcagggaagagtgttctgtgagtacttctgccatacctgttgttaccctgagtgggctgaattacagtgtcaagtgactacgtagcattaaaaagcaagggcgtgttctttttgaaaacacagatacgtaagtaaaactaccatcataaggaagaggcaaatgaatgcaagtgcatctctttcaggcttaggactgaaaccacgctgcagtcacaccaagtgctgcttctgccattagtctctgaatcctacgtgagcataaaaaaaaaaaaaaaaaaatccctgttgtgagaaagctgtgtaagagaccccaaggacagattgcatcattccatgggtaaagagttcagtgaggagtgttctgggactgttgactgccatacctgttgtgagcagcctaattttggtgaaactggaccagttgatgacttcccagtaagaagcggcaagctgcacattccttttggaagactcaaagaatggccgaaagctgttgtcataatgcccagacaaatgagccattgtgtttcttcttggctgtttggaggctgcaactatacatctttcacaggaggaatatactgactttgccatattacccagatttgcagcttcaagataacacatttccctgcacatttattgctgtgtaaagacacttctagtgtacaaattctgtgccgtttgtggagcaaagatagcagtggagagtgctctgtgaatgtggatggccaggcctgttttcaccctgatgaggtgaaaaatactgtcagatgaccttgaaccaagaagtggcaagccgcccgcttcatgagaagtgaagtttctcagtcatgtttgactctttgcaatcccatggactgtaccttaacaggctcctcagtccacggaattttccaggtaacagtactagagtggcttgccatttccttctccagggcatcttcctgacccaggttttgaatgcgggtgttccactttgcaggcagccgttttaccctctgagccaccaggaaagcctgctggggtttacagaaaattcaacttcttaaccaaagctgttctggaatgaaaaagtagaatgagcctgctgtgtttttctggcaggtgggatgctgaaaccacacaggttacccaaggggaaatgactgctttgggacatcagacaccaattccaggtgcacaaaaagatgtctccttgcagagtcaaagctatgtaaatgcattcctaggacacaacgtgttctccgtgaatgttgacttttgcatccctttttgcactgatcctctaaaactgtactgtcagaggaattcccatcaagaagtggcaagcagacttgtagtgctaaagaaggggaaaatgcccccgagagctttctgtcaggctggagattacaagtattccaagttcattagtaacatatgctgctttcatttcagggcctgagttctaaggttcagagaaacatttccccatagcttcagagctgtgcaaagcgactcctatcacacactgtatcattgccttagaaaagagttagggaagaatgctatgtgagtgcctctggcatacctgctgtcaccctcatgggctgaattacactgtcaactgactatgtagcattaatcagcaagtgcctgttctttcaccaaacgcagatatataagctaagctgcggtcatgaggaaaaggcaaatgaacccatgagtgtttctttcagggttaaaagtgcaagcaccctgcagtcacacgaagaaatgctgtgtctgcctttaggcaaaaaatcacaggtgaacataaacaaatatccctgtccttagaaagcagtgtaagacagcccagggacatattgcaccattccatgagtaaagagttcagtgaggagtgttctgggatacctgttgtcagtagcctagtcggtgaaactggactgggtgacgacttcccagtaagaagtggcaagccgcaggttccttttggaagactcaaagaatttcccaaagctgttttcataatgcccaggcaaatgagccagtgcgtttcttcatggctgattgcaggctgcaactatccagctttcacagtagaaatatactgattttgccatataacccagatttccagcttcaagagaacacatttccctgctcatttagtgctatgtaaagacacccctagtgtataaattctgtaccatttgcagaggaaagagcagtggagaatgctctgtggatggccaggcctgtttttaccctgaagaggtgaaaagtactgtcaggtgacctcgaatcaagaagtggcaagcctcccgcttcatgagaagtgaaggtgctcagtcgtgttggctttatgcaatcccatggactgtagctgaccaggctcctctgtccatggaatcttctaagcaagaagactggagtgacttgtcatttctttctccagggaatcttcccgacacaggatcgatccctggtctccctcattgcaggcagacgctttaccctctgagccaccagaggaacccgccagattcactgaaaagtcaaccacttactgaaagctgctctagaatggaagagcagaatgagcctgctgtgtatttctggtaggtgggatgctgcaactacaaaggtttaccaggggaaatgactgctttgggacattagtctccatttccaggtggacaggacgatgtctctgcacttttcaaagctgcgtaaatgcattcctaggacacaatgtgctctccaagaatgttgacttttgcatctgtttttgcactgatcagctaaatctgtaccgtcagaggacttcccatcaagaagtgacaagcagactttggtcttaaagtagggaaaaatgcccctgtgagcttttccggcaggatggagattgcaagtattccaggttctttaggtgcaaatgctgctttcatttcaggccccaagttctaggttcagagaaacggtttgcccagcttcagagctgtgcaaagacactcctagaacacactgtatcattcacttagaaaagagtataaggaatagtgttctgtgattcatctgccatacctattgttaccctgagtgggccaaaatacactatcaggtgactttgtaacattaacaggcaagcacctgtgctttcagaaaatgcacatacttaagcaaagctgtggtcgtaaagaagaggcaatgaacccatggatgtttctttcagggttaaggttgtaaccaccctgcagtgacacaaagaagtgctgcttctgccattaggctaaaaatccctggtgaaccaaacaaaagtccccctgtcattagacagctatgcgactgagtccaaggacagattgcaccattccatgtgtaaagagttcagtggggagtgttctgggactgttgactgccatacatgttgtcagcagcctaattcagtgaaactggactggatgatgacttcccaataggaagtggcaagctgcatgttcctttcagaagactcaaataatggtacaaagctgttgtcataatgctcaaatgagacagtgcatttcttccttgctggttagaggctacaatccaggtttcacaggaggtatatactgattttggcatattacccggatgtgcagcttcaagataatacatttcccagcatttagagctgtgtaagacaccctagtatacaaactctgtaccatttgtggagcaaagacggtagtggagagtgctctgtgcatgtggatggccaggcctgtttttaccctgatgaggtggaaagtactgttgggtgaccttgcagccagaagtggcaagctgcctgcttcatgagaagtgaaattactcagtcgtgtggctttctgcaatcccatggactgcaccttaccagactcctccatccatggaattctccatgcaagagtactggaatggcttgccatttccttctccagagggtcttcctgacccagggatagaacccaagtcccccatgtggcaggcagacactttaccctctgagccactagggaagtgtgccagattcagtgaaaactcaactacttaaccaaagatgttctggaatggaagagcagaatgagcctgttgtgtttttctggcaggtgggatgctgcaaccacacaggtttccccaaggggaaatgactactttgggacttgagaccccaattccagttgcaccagaagacatctccctgcagtttcaaagctatgtaaatgcattcctaggacacagtgtactctccatgaatgttgacttttgtatctctttttgcactgataagctaaaactgtactgttagaaactttccatcatgaaagggtaagccaacttctggtcctaaagaaaggaaatgtgcccccgtgagcttttctgttaggctggagaaacaagtactccatgtttgttaggcgcaaatgctgctttcatttcaggccctgagttctaagttcagagaaacatttccccacagcttcaaagctctgcaaagacactcctagaacaaacagtatcattcacttaaacaagagaccagggaggagtgttctgtgagtgcttctgccataccagttgtttccttgagtgggcagaattacactgtcacatgacttcgtagcaacaataggcaagaggctgtgctttcagaaaatgcagacacggtctgcattttagcaaagctgtgatcctaaggaagaagcaaatgaacccatgtgcatttctttcaggcttaaggctgcaaacaccctgcagtcacatgaagaagtgctgcttctgccattagactccgaaccccatgggagcattaaaaaatgtccctgctgtgagaaagctgtgtaagagaccccaggacacactgcaccattccatggaaaaagagttcagtgggatatgttctcagagttcagtgtggagtgttctgggactgttaactgccatacctgttgtcagcagcctaatttcataaaattggactgggtgatgacttcccagtaagaagtggaaagctgcacgttcctttcggaagactcaaagaatggccgaaagctgttgtcataacgcccagacaaatgagccagtgcatttattcctggctggttggaggctgcaactattcagctttcgcaggacaaatatactgatttttccattttatctggatttgcaccttcaagataacacattcccctgcacatttagtgctgtgtaaagacactcctaatgtacaaactctgtaccgatgtggagcaaagacagtagtggacagtgctctattaaggtggatggccaacctgtttttaccatgatgaggtggaaagtactatctgatgacctcacagtcagaagtggcaagccgcctgtttcatgagaagtgaagtttctcagtcgtgtgcaactctttgcaatcccatggattgtagctaaccaggctcctctgtccatggaattttccaagtagactagagtggcttgccatttccttctccaggggatcttcctgacccagggatcgaacccgggtctcccacaatgcagacagacactttaccctctgaaccatgggtccattgaaaattcagctacttaaccaaagctgttctggaatggaagagtagaatgagcctgttgtctttttctggcaggtgggatgctgcaactacacaggtttcccaaggggaaatgactgctttgggacatcagatcccaattccaggtgcacaagacatttccctgcagtttcaaagctgtgcaaatgcatcctaggacacaatgtgctctctgtaatgctgacttttttatctgctttttcactgatcagctaaaactgtactatcggaggacttcccatcaagaaatggcaagtagcgtgtttctctcagaaaacataagtacttgatgagacttgtggtcctacagtgggggaaaatgcccccgagtgcttttccatcctgatggtgattccaaatatttcaggtttgatgggcacaaacgctgctttcatttcaggccccgagttctaggttcagagaaacgtttccccacagcttcagatctgtgcaaagacactcttagaacacactgttaccattcccttagaaaacagaccagggaagagtgttctgtgagtgcttctgccataccagttgttatcctgagtgggcagaattacactgtcacgtgactttgtgacattaataagcaagcgcctgtatttcagaaaatgcagacactttagcaaagttgcctttgtaaggaagaggcaaatgaacccatgtgcgtttctttcaggcttaagattgcaaccaccctgcagtacacgaagaaatgctgcttctccctaggctccaaatcccatgtgaaccaaatgtccctgcaaatagggaaaaatcagtatgttcctcctgggaaagcaggagagttgtagcctccagccagccacgaaaacaccccagggcattccattgctcattatgacaacagctttggtcaattatatgagtttgctgagaggaacatgctacttgccactgcttactgggagttatcacccagtccactctcaaagaaataggctaaccacaggtatggcagtcagcaggcacagaaccctccccactgaactctcagaaccctccccactcaactcttaactcttggaatggtgcagtctgtccctgggctctgtgacacagctctctaactacagggacgtttgtcttcttaaaacgggatttggagtctaatagcagaagcagcatttcttcgtgtgactgcagggtgcttgcggtccttagcctgaaagaaacacacatgagttcatttttctcttcctgaagaccggagctttgctaaagtatctgcgttttctgaaaacataggcacttgcatattaatgctacaaagtcattggacagggtcctttggcccactcagggtagcaagaggtatggcagaagggctcacagaacactcttccctgaactcttttctaagggaatggtacagtgtattctaggagtgtctttccccagctctgaagctgtgggaaaaacatcttactgaacctagaactcggagcctgaagagaaagcaaccttttcccctaacaaaacttgaatacttgcagtctccaacctgacagaacaccacactggtgcatttcccccttcttcatgacagcaagtttcatcaagtacttgtgttttctgggagaatcacgcttcttgccttttttttgtgggaaagtcctctgacagtacagttttaactgttcgggataaaaataggcagagaagtcaacattcacggatagtacattgtgtcctaggaacggactcactcagctttgaaactgcagggagacgtgtgttttactacctgggattggcttagggcatcaaacagcagtcatcttctgtgaaacctgtatcattgcagcaaccaacctgccagcaaacccacaggctcattctattcttcagtcctagaacagcgttggttaaggagctgagtttttagtgaatcaggcgggcttcccggtggctcagggggtagaacatctgcctgcaatgcgggacacctgggtttgatccctgggtcgggaagttccacccaccagagaaggaaatggcaacccatttcagtactcttgcctggaagctacagtccatgggaaagagtcgaacacaacttagcagcttcatatctcatgaatcaggctgcttgccacttcttgctgtgagctcatctgacagtacatttcacctaatcagagtaaaacagccttggccatccacattcacagagccctctccatttatatctttgctcagcacatggtacagtgtatgtacactaggagtgttaacatgggatttggagcctaatggcagaagcagcatttctttgtgtgactgcaggtggcttgcaactccttgcatcaaggtcctttccattggaatgtcaactcatctaggtaacaacactatggcactcagcactctcaaatactctcccacagattttcctcatgggcagtcccagtatgaccaaggaatctgttcactagtctcagaaagtggagggaaacatactggctgtgactctcaaaattagggcctgtgtgcaaatgtggtgttctcacatcataaaatctagacagctgctgagagagagatgcctgaaaatcactcttttttcgcctttcatgcatcaaagcactgtacgtttcccagcgttctgtttgctgagagtaacccactacttgtgaatccttgcttggaggtaacaatttcccctcacctgtttcacagtacagaaggaaagcataatcgctgctgcggccgctaagtcgcttcagtcatgtctgactgtgcaaccccatagacggcagcccaccaggctcctctgtccctgggattctccaggcaagaatattggagtgggttgccacttcttctccaaagcataatcacagggagaaacaagacaaaaaccccaagttccaacaacttttctctgtaggaaggatccagtgatcctaggtctgtcttgaaaattcttggatactgcagggatacctactgagtgtgaacctggatattgcaccttgatgacaaaattagcattgtctcttggttgactctagaaggctggggtttagcaactgcctgaaaatctatccaatgttgttttcgtactttgacagcaatagctttggttaaaaacattgggatttctgaagcaagtaggcagcttataagttcttgtgtcaaggtcctttccaccagaatgtcaatgaatctaggtaacaacagtatggcactcagcactctcagtcactctcgcgcagactgtttcttcatgggcagtcccaggatgacctaggaatgtattcaccaggctcagaaagtgcagagaaacataccagctgtgactgtccaaattacagcctttgtgcaaacgtggtcttctcatatcatagaatctagacagttgctaacaggtaggtgcctgaaaaacactccccttttttgcctttcattcatcaaagaaacataggtttcccaacattctgtttgctgagaataacaacactacttgcaaatctgtgcttggaggtcattttaactaacaattacccctcacgtgtgtcacagtagggaaggactgcctacgtacagggaaaaacaaacaagccaaaaaactcaagttccttacattgcctctgtagaaaagatccagtgatcctaggtctgtcagggacacacactgcgtgtgaacctggatactgaaccttgctggcaaaattagcattttctctcttatggccctgaaatggtgcagcctagcaactgcctgaaaatccatacaattttttcccctacttttgacaccaatagctttggtcaaaaattgggatttctgaagcaagcagggggcttgcaactccttgcgtcaaggtcctttcctttgggatatcagctaatctaggtaacagtggtatggcactcagcactctcagatactctcgcacagactgtttcctcagggatgtcacaatatgacctaggcatgtgttcacaaggctcagaaagggcagggaaataaactggctgtgactctcgaaattacatcaggagggatgctgcaactgcacagggttcccagggggaaacgactgctctggggcatcagatcccaattccaggtgcacaagaagacgtctccctgcagtttcaaggctgtgtaaatgcattcctaggacacagtgtgctcttcgtgaatggtgacttttatacgtttttgcactgatcagctaaaactgtactatcagaggacttcccatcaagaagtagcaagcagtgtgtttctctcagaaaacacaagtacttgatgagacttgcggtcctacagcagggaaaattgctcctgtgtgtttttccatcaggctggagattgcaagtattccaggttcattaggcgcaaaatgctgctttcatttcagaccccgagttctaggttcaaagaaacatttccccacagcttcagagctgggcaaagatacccctagaacacactgtatcattctcttaggaaagagaccagggaagagtgttctgtgagttcttctgccatacctgttgttaccctgagtgggcagaattacactgtcgggtgatttgaggcattaataagcaagtgcctgtgttttcagaaaacgcagacactttagcaaagttgccattgtaaggaaaaggcaaatgaacccatgtggttttccttcaggcttaagactgcacccaccctgcagtgacggaagtgctacttctgccattaggctccaaaccccatgtgaacataaacaaatgtccctgtcattataaagctgtgtaagagaactcaaggacagactgcatcatttcatgagtaaagagttcagaagggaatgttctgggagtgttgactgccatacctgttgtcagcagcctaatttggtgaaactggactgggtgatggcttcccagtaagaagtggcaagttgcatgttccttttggaagaattaagtaatgaaccaatgctgttgttttaatgcccagacaaatgagccagtgcgtttcttcctgtctggttggaagctgcaattatccagctttcacaggaggaatatactgattttgccgtattatctggatttgcggcttcaagataacacatttccctgcacatttacagctgtgtaaagacactcctagtgtacacactctaccatttgtggagcaaagacagcagtggagagtgctctgtgaatgtggatggttaggcctgtctttaccatgatgaggtgaaaagtcctgtcaggtgacttcacagccagaagtagcaagcagcctgcttcatgagaattaacgttgctcagtcgtgtgcaactctttgcaatcccatggactgtagcttaccggctcctccatccacggaattttcctggcaagagtactgaagtggctttccatttcctactccaggggatcttcccgacttggggatcaaacctgggtctcccaggttgcaggcagatgttttttcctctgagccaccagggaagccagccagattcattgaaaattcaactacttagccaaagatttctggaatggaagatagactgagcctgttgtcctttttgggcaggtgggatgctggaactacgcaggtttcccatggggaaattacttctttgggacatcagaccccaactccaggtgcacaaaaagatgtctccctgcagtttcaaagttgtgtaaatgcatcctaggacacaatgtgctatccttgaatgttgactttcatatctgtttttgcactgatcagctaaaactgtactgtcagaggacttcccatcaaaaagtggtaagcagcatgtttcttcagaaaacacaagtacttgaggagccttgtggtcctgatgaagcaaaaaaatgtccccgtgtgcttttctatcaggctggagattgcaagtatttcaggttcgctaggtgcaaatgctgctttcatttcagcccccaagttctaggttcagagaattgtttccccacagcttcagagctgtgcaaagacactcctaggacagacacactgtatcatttccttagaagagttcagggaagtgtgttctgtaagtgcttctgccataccctttttttaccctgagtgggcagaattacactgtctggtgacttgttaacattagtaagcaagcaccttgccttcagaaaacacagatacttaagcagaactgtggtcataaggaagaggcaaatgaacccatgtgcatttctttcaggcttaagattgcaaccaccctgcagtgacacgaagtgctgcttctgccattaggctcagaaccctaagtaaacataaacaaatgtccctgtccttagaaagctaatgttggtccagtggtttgtgtaagctttgtataggctgagacttatgctgagtttttgtttgtttttcctctgatgggcaaggctgagtgattgggtttgtacttttgttttgtttgttgtttagatgaggagtcctgcacagggtgctactggtggtctggtgatgccagggcttgtattcaagtggtttcctttgtgtgagttcacactatttgatactctctaggattagttctctggttgtctagggtcttggagttagtgctcccactccaaacgcttagggattgatctctctccaaagaccagcttaggtccaaactaccaagaggaatttcacttgaaatgaaagggcctttactttaccaagaggaatttcacttgaaatgaaagggcctttactttaccaagaggaattcactcgaaatgaaagggacttactgaattccaaaagccagagcacaagaacacatggagatctctacccagaggaaactctaccatgccttggttgcagcagatgttggtcctcttctgctttgatctgtccccgctttctgtggcagctgcccagcagaggccatctgacgtcattttgttcattacctggatgaaggggtgtctccttctcagaggagcctgagacaaacaaagggacagtgcagagccatccccgcacggaagcccccttccatttagaaatgtttctcttaagctatgttaatgaactatgtatttagcctagactctgtgtttcttcacttaggttctgcctaagactcagaactgataatggctcaacaaaccagtatgtttttctcatacaattgttctcctaatctatgttaatgagactatgtatttgcttggaaacctgccttcttcaaaattcatgtcaatcattttatggcctgggatgactcaccttgttccaatgttacctcaaaatgcatgttgtgggtgaggggccctggtgccactctctgagttttgagacatttcctttctttaattagtacccttctgataggtgtataagttaccattaaagtctagcagggggggcactctttctgccccttctaatgtctatgttagaagcttaatctcttttatactttaataaaactttatcacacaaaagcttggagtgatcaagtctcataactggccccagattgaattcttctcctccaaaggccaataatcccatcatctttcatggctcagcaacaacctttcaccgggggagctcatctgggattcttcaggacaaggtaaggacacttggagctctagttctttgttctcctggcaaacacattttctgctgtactttactaactctatggtgtgcttgtgtgtgtgattgaaagatgcacacatgtgtgaagcaagatctgggtccaaatctttggttctgtggtgacctcataccacttatggcaggaaccctgttgggggattatactgacctgctaatgtcaagaggcacccaatgtctcctccagggaaacagaccaaggtggataaaacgtgtggatggaactctccttttttggccaaactttctggtctctttgaccatttcataacttcctgggaattagaactactaacctaatcagtgggatcatagactttcaagggacttgttatctatgctgttactgtgtattgtcacttaggttccaaactttgttttgtttttttttgtattcacaaattgcctagcctcactaggagtcaatagtttggaagctagatggagttctaattccaagaacatctctcaggtttaagattactcaggaaatacagcagtcttcttcctttggtaacactagctcttagtggaccagaggaggatctccagttgcttctgtctcaacaccttagatattcttcctgtggaatctgtgggaatgaactggaaggactggccataataacttgaggacaaaaatcactttttcctcagtggccagcccctcaccgtctcttttgctatcgcttatattggtgtggtgagactcagaaggaacatcttggttttatgtttgtcctttatgatttactggtcttactgtggtcaggaatgtactcagggttgtgcataggcactcaggagacaaatatttcccttagtggtcttagcttgggaggcattctggaaggttactctgactgcacctcgggtggcatcagaggcaagcaaaagttttaatggtgaggaactgggtattagtctgggatgccatcaggtctacccctgatgcatctccaccccaccgcagtggtagaatggggaggggcagtagtggaatacctgtggtaagagacaggttaactccagccagggaaggaagcttaggtggaagacctgtctccacccccatctagaacagggagggacagtagaaggacagtgctggtagctgtttttctctcttaaaggtgggagctaaccattccagcctcactcctttgaaaaactgggatagatttgatccccagagcctaatgaagacatgcctgatcttcctatgtgatactacatggccacagtatccattggaggatagcgaatggtggctggttggagggtctcttaattacaatattgttttacaattaaactggttctgtagataacaaggaaatgggtagaagtagcatatgtgttgccctttttctctctgtgagacatatcagatttatgtcctaagggtatatattagggtatgaaattttcagctccctattctgctatatgaccttatttgggaggatgtgatgtatatcctgggacaggcgctaactcctgcatcaacaacttgagtttggaaagctgttgcctatggagatgaatggcttggcaaggaatcattagggaagagggaggatgagatagctgccctccccactggggatcaggcagtcccaactatagaaccagattgggactaaaaggctaaaggatgatgggataagagtcatttgtcagatgtgttcttgaaggactcagacaagctcatgctaagactttaaatgatgctaatttggcaaacatagaacaggaagagaacgaagcttctggtaaattcctagatagactgagggaagccctttgcagattcactgagattgatcctagtcagctccagatatccactaaaacacatgtatggaccaaatcagtctttagataatctgttgcaattggctcagtcagtctattatggcagggagtatgagggaagaaagaaaggcagagaaagaccaaggtactggctgaagcccttgtaatggctgtcaggactgttcttaaacagcctgagaaaaattccaggagagacccaggtgaaaggggatgggcttgctatttctgtggaaaggaggagcgcctcaagcgggattgccctcaggcatctaaggggtccccagctccatgtttgcctgtaaggggccacactggaggagagactgcccccagacgcgtaggtcccagtggtgggattctcaagacaaccaggactgaatgtgcccaggggtccccacacaaactcccaccctaattacagctgaggaaccccaggtattagtaactgtgggtggccaatctgtcaatttccttgtggacaccagggcaagttactctgtgcttactgaagcccctggtccactttctccccaatccgcttctataatgggactgtctggacaagccaaacattaatattttggtcatcctctaagctgtcaactgggactctgtttttacagagtgccagattgtgccagagtctccctcaccccttttagggagggatatactgagcaaggtccatgcctctgtttcatgaatatggagcccttcctttctctccctttaattgaacaaaatgtaaatcctaaagtgtgagctgatggaaaatctgtgggtcgaacacaaaatgctattcctgtagttgtgaagctcaaaaacccactcatacttccccatcaaaagcagtatccactgaaacccgaggttaaagaagggttaaaacccatcatcgagattttaaaggagcaggggctattaattccctataacagtccatgcaacactcctattttgggtataaagaagtcaaattataagtggagactagttcaagatttacaaataataaatgaggctgtacatcctttacaccccatggtgcctaatccttatactctattgtataaaattactgaacaagagaaatatttttcagcatgctttctagagttaaacctatactatgtcatcccctacctatgactttcagacaattgagaggatttgggggaatcataggctactgcctcatttggattctgggttatggggaacttgcctggcctatatatgaacttacaactgaaactcaacaagcccaaactgacaaactggttcagtctctagatactcaaaaggcttttaaagctcttcagattgctctcctgccagctcctgctttaagcttgcccacagggtcagaatttaatttgtttgtcactgaaagaaaaggtatggtcttgggagttttgacacaaccccgagggcctcatcagctatatataggatacctgaaaaacttaaatcctgccactttccttcctgacaaggaaaatgaaacacctgatagcaattgttcccaatttctaactttaaactattcagctcgggaagacctgatggataccccattagacaatcctgatatggaattatttacagatggcagttcttttgttcgggatgggaaacttaaagcaggttacactataatgcgactggacagattttaaaagcaaagtctctccccagggaatgagcgctcaggtagtggagcttgtggctctgacccgagctctagagttaatcaaagggcagcaagtcaatatctacagtgattctaagtatgcttatttgactttacatgttcatgctgtgatacggaaagaaagacagtttaaaacggcaacaggagaacctattaagcatttcaaaagactgagggacttttaactgctataaattgtcctacagaagtagctgttgtgcactgcaaaggacacagtagggatgggaataagtagctgagggtaatcagctggctgactgtcaagccagaaaaccagcagtttaagaaaccccttcactgcagatgcctttgaactagacaggtcctgtggaataggaaaaacatcacaatgaggaagaattagaaagatatgagaaagtaggagcaaacattatcgataaaggatgcttatagtccaaggatggatgatgaataattactgaaaattctcaatggaaaattcttaagagtttacaccagagttttcatttaggtgttgagagcacttaccagatggcttctcatttctttgaaggaaaaatgtaatggaaactttagagaacattatcaaaaactgtgagatttgtcagaaaaataacccaaagactgaaaagttagcaaaatctgggttacaatgaagtggaaaatatcctggagaggactgggaaattgattttactcatatgccaaaggcaaatggatattcttgattacaagtttgggtggatatatttactggacagattgaggcttttccctgtcatagtgaacagcctaagcaggttataagaattttaatccatgaaattactcccaggcttgggctgctgtggagccttcagagtgacaatggctttgcctttaaagccactgtaactcaggggatgtcaaaagctctaggaatagacgatcacttacacggctcctggagaccccgatcctcaggaaaggttgaaaaagctaatgacattattaagagacatctgtgcaaattaactcaagagaggcatgacagttggtgtaaagttctacacatagctttaatgagggctcgaattgccccccaaaatgagggactgtccccctttgagtgcatttatggaagacccttcttacccacagacattgttatagaccttgaagccttggaattatctaactgtgtaactcagcactcagcttttcaacaggcattaaaggaactctgatgtgactcatgacccagactctaagtcaagaaagacactgtctgagccaggaactgaggtcctgataaaaatattgggatctcgggggcaatccctggagcccctctgggaaggcctttaccaggttattctatcttttcccatagctgtcaaagtgccagatattgatcattttacaccacacttaagagttggcatcctgaccagaactaaatgatgtcactttatgtctttattctctacactcttactttgtacttttcagatcagcctgataatctatgtgagcttgcttctgctgactccaaaaatccagtgtctgccgtttgaccctcaagacaatgccctcctgtcctgggatcactcctatgctgcatttcacattcagtctaattactgggtctgtggagcactcccttcttcatcagtggaaggcttccgtggtgggcatctccacttgaaggaaaggagtttcttcaagtctgcaaatctttctacaaagacaataatatgtgatgcctcttcttaatatgataacatctaacaatcctaagatggactggtgcaacactttgtaccttaactatgggcactatgagacttttaactttgctgattgttctgtttttgctgtttgctccctgcatctgtaagagtgtggctggatttgtttctagctgcatgaaggattttaagtgacaaatggttgctcaaactcctgccactgtggcagcttcctccaactacctacttggggcccctggatcagagaccctcaatatgagggttaggagagtatgttgcctcaccaatttagggacgatgccccttatcagcttggaagcagttacagaatgaaaacaatgcccctttccctaggaaacataattctcctaaaagaaaagggagaaataagacggtaacaggcaggaaggctcagttcagttcagttcagtcactcagtcgtgtctgactctttgcgaccccatgaattgcagcatgccaggcctccctgtccaccaccaactcccagagttcactcaaactcatgttcttcgagtcggtgatgccatccagccatctcatcctctgtcgtccccttctcctcctgcccccaatcccttccagcatcagagtcttttccaatgagtcaacccttcacgtgaggtggccaaactattggagtttcaacttcagcatcagtccttccaatgaacacccaggaccaatctcctttagaatggactggttggatctccttgcagtccacaggactctcaagagtcataggttgcaaatgtcagacatttttcatctctctctcaagtggcaggaggaaacaaactgcaagtgtcagatttcttttcccttctctatacaaaattaaaagatgctttcttttaaaattctgtgttgccatgacacctggttccacctgaacttaacttttctcaaatcttgagccaaccaatgcatttttcttatggaaatgtttttcttaagctatgttaatgactatgtatttaacccctagactccgtgtttcttcaagtcggtttcacctaagactcagaaccgataatgactcaacaaaccagtatgttttactcatacagttgttctcttaatctatgttaatgagactgtgtatttgattggaaacctgcctttcttcatgccaatcgtcttatggcccaggaagattcaccttgtgccaatgttatctcaaaatgcatgttgtggctgagtggcctgcagccactctctgaattttgatacatttcctttctctaattagtagcctgctgatatgtatataacttactgctgaagactagcaggggggcactcttcctgcccccttctcttttcctctggaggccaagaactctggtgtctttcattgctcagcaacaacctttcaataggaggatactgatatttctccaagaaaagctatagttttgactatacagacctttgttggcaaagtgatgtctctgctttttaatatgctgtctaggtttgtcatagctttcttcccaaggaacatctttttttttaatttaatgactgcatttttaatgttgttatgctttggtccagctgttgcattctttctgaagctattagtaattaccctctgctctttatcagtagcttattcgacactttctgacctgaggggctcatcttccagtgtcatctatttttgccttttcataacatttatggggtttgggcagcaagaatactggaggaaattcccatttcctccttcagtggaccatgttttcccagaatacttcaaatgacctgtccatttttggtggccctgcatggcatggctaatagcttaattgagttatgcgagacccattgccgcgacagtgctgtgatccatgaagagacaggaagctcttagaattactttctttttaatgcattctatttattcccctcagctagattctaagtgtaatttgtctgtttattcattgatacatttaacagatgtagaagagttccttttgtttctaaaatattcaaaatatttctttatatataaaggatattcatgattttgtaataattttcacaaactgacataataattttactgttcagttcagttcagttcagtcgctcagtcgtgtccgactctttgcgaccccatgaatcgcagcacgccaggcctccctgtccatcaccaactgccggagttcacccagactcacatccattgagtcagtgatgtcatccagccatctcatcctctatcatccccttctcctcctgccgccaatccttcccagcatcagagtcttttacaatcagtcaactcttctcatgaggtggccaaagtattggagtttcagctttagcatcattccttccaaagaaatcccagggctgatctccttcagaatggactggttggatctccttgtagtccaagggactctcaagagtcttctccaacaccacagttcaaaagcatcaattcttcggagttcagctttcttcacagtccaactctcacatccatacatgaccactggaaaaaccatagccttgactagacggatctttgttggcaaagtaatgtctctacttttcaatatggtatctaggttggtcataactttttactgtagactacttttttttttttgagatggcaagaatacacagaagaactgtacaaaaaagatcttcacgacccagataatcatgatggtgtgatcactcacctagagccagacatcctggaatgtgaagtcaagtgggccttagaaagcatcactacgaacaaagctagtggaggtgatggaattccagttgagctattccaaatcctgaaagatgatgctgtgaaagtgctgcactcaatatgccagcaaatttggaaaactcagcagtcccacaggactggaaaatgtcagttttcattccaatctcaaagaaaggcaatgccaaagaatgctcaaactaccgcacaattgcactcatctcacacgctagtaagtaatgctcaaaattctccaagccaggcttcagcaatatgtgaactgtgaacttcctgatgttcaaggtggttttagaaaaggcagaggaaccagagatcaaattgccaacatctgctggatcatggaaaaagcaaaagagttccagaaaagcatctatttctactttattgactatgccaaggcctttgactgtgtggatcacaataaactgtggaaaattctgaaagagatgggaataccagaccacctgatctgcctcttgagaaatttatatgcaggtcaggaagcaacagttagaactggacatggaacaacagactggttccaaataggaaaaggagtatgtcaaggctgtatattgtcaccctgcttatttaacttatatgcagagtacatcatgagaaacgctggactggaagaaacacaagctggaatcaagattgccaggagaaatatcaataacctcagatattcagatgacaccacccttatggcagaaagtgaagaggaactaaaaagcctcttgaggaaagtgaaagtggagagtaaacaagttggcttaaagctcaacattcagaaaacgaagatcatggcatctggtcccaccacttcatgggaaatagatggggaaacagtggaaacagtgtcagactttatttttctgggctccaaaatcactacagatggtgactgcagccatgaagttaaaagacgcttgctccttggatggaaagttatgaccaacctagatagcatattcaaaacagagacgttactttgccaacaaaagttcgtctagtcaaggctatggttttcctgtggtcatgtatggatgtgagagttggactgtgaagaaggctgagcgctgaagaattgatgcttttgaactgtggtgttggagaagactcttgagagtcccttggactacaaggagatccaaccagtccatcctgaaggacatcagccctgggatttctttggaaggaatgatgctaaagctgaaactccagtactttggccacctcatgtgaagagttgactcattggaaaagactctgatgctgggagggattgggggcaggaggagaaggggacgacagaggatgagatggctggatggcatcactgacttgatggatgtgagtctgagtgaactccgggagttggtgatggacagggaggcctggcgtgctgtgattcatggggtcgcaaagagtcggacgtgactgagagactgatctttttttttgtagactacttttaattcaaagaaatgtccgtcaattattttcttatgatcacctcaactttgtatctatggtaagcacagaaaagttcaaaactttacctcagcatttcctattatatttcttccttgtgtataagtcaataatatgtgattctagccaaatgcacaaactgttcagtcatcaaagcacttactaggtgcctgatactgcagtaggcatgatgggaagcaacatacatgcatcacagagggacatgctaaatattgttgatatacattaaaggaatagttagggaaaatatcgatataaaggaaatggtaaatctgatggagtttatagaggattgtgtattgtcaaatacagatgtcaatttctataagtttaacatataaaaatggagaacaaagggctacaatgagaagatataaacattaaaatcatctagcaaatgttatctcacaattaaaaaataccttctgtgcactaaagcactaaacttatttttcttaaagcccttgaaattaaaggctataaaccagttctttcaaagggtaaacaaaattgataaactttaagccagactcatcaagaaaaaaaaagagatggaaaagaacccaatgaataaaatcagaattgaaaaaggagaaggtacaacagatataacagaaaccagtggactataccccaatcaaatgaaaaccctagaagaaatggacaaattcttagaaatatacaatctccaaagactaaaccagtcagaaatagaaaatatgaacagaccaattaccagtaatgaaattaatcagtaattttaacactccccaaaaaataaaagtccaggacaagacggcttcacaggtgaattctatcaatttaacaaacagttagcacctatatttctgaaaccatttcaaaaaattacagtgagagaaacacttccaaacacaatctaaatgccaccatcaccttgatattaaaatcagaaatatgccacaaaaacaaataaaattacagtccagtaacactgatgacagagaaggcaatggcaccccactccagtactcttgcctggaaaatcccgtggatggaggagcctggtaggctgtagtccatggggtcgctaagagtccgacacgactgagcgacttcaccttcatttttcactttcctgcattggagaaggaaacggcaacccactccagtgttcttgcctggagaatcccagggacgggggaccctggtgggctgctgtctatggggtcgcacaagtcggacacgactgaagtgacttagcagcagcaacactgatgaacatatacacagaaaccaccacaaaatacttgcaaaccaaatccaacaatacattaaaaacaccagacaccatggtgaagtgggatttatattagggatgcaaggattttttaatatctacaaatcaatcattagaaaatttgaaaaatgaaagcaacctgattatctcaataggtgagataaaaaaaaaaggttttaaaaaattcaatccccacttatgattaaaaaaaaaaccaataaaaggacatgtggggaacctacctaaatatgataaggatcatatacaacaaactcacagcaaaaatcattctcaatgctgaaaattaaaaggcatttcctctgagatcaggaaaaagacaatgatgttcattctcaccatttttatttagcatagtttgggaaattctagtcatgggaatcagagaaaaaaattaacaaaaagaatgcaaattaaaaaagagtaagtatggactctgttgcagagggagagggtgggaagatttgggagaatggcattgaaacatgtataatatcatgtatgaaacgagttgccagtccagattcgatgcatgatactggatgcttggggctagtgcactgggacgacccagagggatggtatggggagggaggagggaggagggttcaggatggggaacacatgtatacctgtggcagattcattttgatatttggcaaaactaatacaatttgtaaagtttaaagataaaaaaattaaaaaaagagtaagtaaaacaatcactgtttgtagatgacatgatactatacatgtgtgtgtactaggtcacttcagttatgtttgactctttttgatcctatggactgtatcccaacagtctcccctgttcatgggattctccaagcaagaacactggagtgggctgctgaaaccttctgcaagggatggtcatgatctagggactgaactcgcctctcttacatctcctgcattggcaggcaggttctttaccactagcgccacctgggaagacaaatactatacatacaaaatactaaagacaattccagaaaactaaaacagctaatcaatgaattcagtgaggtttcaggatatggaattaatacacagatttcccttgtattctgatacatgaaaagtaaaagatccataaaaaattaaggtaacaattccacttaccatcatatcaaaaagaataaaacacctaggaataaacttacctaatgaggcaaaagacctatactcagaaaacgataagatactgataaaagaaatcaaagatgatacagatggagaaatataacaagtttttgagttggaagaatcaatgctgttaaaatgactgtactacccaaagcaatctacagattcaatgccatccctgtcaaatcaccagtgacatttatcccacaattagaacaaaatattttttacactttgtattgaaacacaaaagaacccaaagagccacaccaatcttgtgagagaaaaaaaggagctgaaggaatcaagcttcctgacgtcaaattatgctacaaaagaagagtcatcaaaactatatgatactggcacaaaaacagacatatagatcaatgacacaggatgagaccccataaataaacccacattcttacggccaattaacctatgacaaagaaggcaagaatatacaatgaagaaaagacactatttgcaataagttgtgctggaacaaattgacaactatatgcaaaagaaaaaattagaatattctctaacatcatgtataaaaataaagtcaaaatgggtcaaatacctaagtataaggctagatacttgaaaaatcttagagtaaaacacaggtagaacataaattgcagcaatatctatatttagatatgtatcctggagaaatgaaaataagaaaaacaggcaaacgggaccaaatgaaacttaaaatcatttgcaaagcaaaggaagccataaacaaagtgaaaagacaaaccagagaatgttagaaaatatttgtaaatcaaatgattgataagagattaatttccaacatatacaaagggcacatgtagctcaacagaaaacaaaacaacacaatcaaaaacagactattcagttcagttcagttcagtcgctcagtcatgtctgactctgcaaccccatgaaccacagcacaccagacttccctgtccatcaccaactcccggagtttacccaaactcatgtccattgagtcagtgatgccatccaaccatctcatcctctgttgtccccttctcctcctgccctcaatctttcccagcatcagggtcttttcaaatgagtcagcccttccgcataaagtagccaaagtattggagtttcagcttcaacatcagtccttccaatgaacacccagaactgatttccttcaggatggactggttggatctgcttgtagtccaagggactctcaagagtcttctccaacaccacagtgcaaaagcatcaattctttggtgctcagctttctttatagtccaactctcacatccatacatgactactggaaaaaccatagccttgactagatggacctttgttgacatagtaatacctctgctttttaatatgctgcctaggttggtcataactttccttccaagaagtaagagtcttttaatttcatggctgcagtcacatctgcagtgattttggagcccaaagaaataaagtctctcactgttttcattgtttccccatctatttgccatgaagtgataggaccggatgccatgatcttagttttctgaatgttgaactttaagccaaccttttcactttcctctttcactttcatcaagaggctctttagttcttcttcattttctgccataagggtggtatcatctgcatatatgaggttactgatttttctcccagcaatcttgattccagcttgtgcttcttccagcccagtgaagaatagcaaggagcgataaagccttcctcagtgatcaatgcaaagaaacacaggaaaacaatggaatgggaaagactagagatctcttcaagaaaattagagataccaagggaacatttcagacaaagatgggctcaataaaggacagaaatggtatgggcctaacagaagcagaagatattaagaaaaggtggtaagaatacatagaagaactgtacaaaaaagatcttcatgacccagataatcacggtggtgtgatcacccacctagagccagacatcctggaatgtgaagtcaagtgggccttaggaagcatcactaccaacaaagctagtggaggtgaaggaattccagttgagctatttcaaattctaaaagatgatgctgtgaaagtgctgcactcaatatgccagcaaattgggaaaactcagcagtggccacaggactggaaaaggtcagtttgcattccaatcccaaagaaaggaaatgccaaagaatgctcaaactaccacacgattgcactcatctcacacgctagtaaagtaatgctcaaaattctccaagccaggcttcagcaatatgtgaactgtgaacttcctgatgttcaagctggttttagaaaaggcagaggaaccagagatcaaattaccaacatccgctggatcatggaaaaagcaagagagttccagaaaaacatccatttctggtttattgactatgccaaagcctttgactgtgtggatcacaataaactgtggaaaattctgaaagacatgggaataccagaccacctgatctgcctcttgagaaacctgtatgcaggtcaggaagcaacagttagaactggacatggaacaacagactggttccaaataggaaaaggagtacgtcaaggctgtatattgtcaccctgcttatttaacttatatgcagagtacatcatgagaaatgctgggctggaggaagcacaagctggaatcaagattgccgggagaaatatcacctcagatatgcagatgacaccactcttatggcagaaagtgaagaggaactaaagagcctcttgatgaaagtgaaagaggatatggcatcaccgactcaacagacatgaggttgtgcaagctccgagagttggttatggacagggaagcctggcttgctgctgtccatggggttgcaaagagttgaccatgactgagcgactgaactgaactgattaattagtgatattcagtatctttacatatctttagtggccatctgtgtatcttcttttgaggaatgtttatttagatcatcagtccatttttggcattgcctttctttgggattggaatgaaactgaccttttccagtcctgtggccactgctgagttttacaaatttgctggcatattgagtgcagcactttcacagcatcatctttcaggatttggaatagctcaactggaattccatcacctccactagctttgttcgtagtgatgctttctaaggcccataaccaaagctaaaacaacactcaggtgttgatgtgactggtgatggaagtaaagtccgatgctgtaaagaacaatgttgcctaggaacctggaattttaggtccatgaatcaaagtaaattggaaatggtcaaaaagaagatggcaagaatgaacatcaattattttaggggtcagtgaactaaaatggactgtaatgggtgaatttaactcagttgaccattgtatctacttactgtgggcaagaatcccttagaagaaatggagtagccctaatagtcaacaaaagagtccaaaatgcagtttttgggtacaatctcaaaaacaacagaatgacctctctttgtttccaaggtaaacattatcacagtaatccaagtctatgccccaaccagtaatgctgaagaagctgatgttgaacggttctatgaagaccattatggagaactgccccaggagccctgactctccacgctttgtgggtgctcctatcggagacagggcaagttgagacatagctagagaaagacctgaggcagagacaagagatgcaggccttgaggtgggaaggtgtcagtgttctggaagcctgcaacaggtgaactcaagtgggccaagaaaatgcaagacgaggtctcaacagcatctgttccaagtctattgagaggtacacaaaacaatctgagtaagctgattcatgttattttcctggatacgcaaacaatgtcttagctcaggctgctatgacaaagtaccacagactgagtggcttaaagaacacaaacatttctcagtatttatcatgctttgaattctatttgtgcaatgtttgaggggaatgcagtgcttatctttttaataggtacattaactgtgtcttcgtttggatgtaccaaaggatgagacaatgggagatgtgagttggtggatcgatgcctcagcttccccttcttgcagctggatgatctgaggtgtattcccattatttcacagatggtccctgtggcatcaagctccactcacctaccatggtaatcggcccacttttcctgactttgctcccttccttgtctcatgttttctacttcctcactttgcttttctggaggctgggaaaaataagatcttggtattggtcaatccagttactggtgaggactctcttcctggtttgcagacagctgccttcttgctatgttcttatttggctgagacagcaatcatctctcatgtctcttctcataatgacactgatacaggagatagatgggctccagtttagacatttataactggcctcctgtttgcattttatggggcagaaaaaagtgggcttcaggctggacacttacaactagccttctctttggatttcctggacaaggataggtgggctctgggtaaggcacttacaaccagccttctgtttgctctccaaaatggaagtaacaatagaaacagagtaaatagccagattttgtctcttgtcaatatcttaaaacaatagtcgtggcaagaacaaagaggggtaaaatcctatttgagtaaaggattaaaggctctctgctcccctccttcttgggacaagggagacactacacatgcacagaaaggctacttgggagacaaaagtcagaggaaatgccaggccataatgagtttcccctcccaaatgctttcaagtcagttcattttggctgaggggtgcatgaacacgtaaggggagggtcctgagacaaattagctggggggacaaaacaagatgattagcctgagggaagaaaaagacctggaaaactccccccttataaaggatttaaacttcccaaaggcatgactcttctctgagcttccctgtgcatcttttcacatgtatttttccaataaaatttttacttttctcattaccttctgcctcctcacctgaattctttcttgacgagacaggcatggactatcgacccaggctctagccaactggcctttgtggtctaatggttaggactcctgatctgggaactaagatcttgctccctgctactgctctctgctgcttgctgcaaggggttgcttactgatgctagcatctgaaatcaacactaagctcatccatgagggttctaccctcacgatctaatcattcccaaaggtcccatcttcatactggggataagggttcaatgagtaaatttgggagggacacaaacattcagtccacagtacataaagatgtcaatcttgcacatatttatttctaaatacaatgccattccaataaaaattccaactacaggtttcttggaattcaacagaattatataattaacctggacagaaaaactaagaatattagagagattctaaagcaaagagaggatagtggttgatattagtcccattaagtactgaggcatatgataaagcttttacccacttcatgtattagtatttatgtgggtttcctagagcatgaggaaacaaacctcttcccatgtagcaccccttcctccagttgtaacaacttgaattactctgattaattgtggagctatgtaaaaagaaagcaggtatatccaccaagtagggtagcagcttactgttcaccaatgtgacactgtgctttacaacagtcatccagaagatcacaatacatttctcatatatatttagtcatctactgctcttgattcacagctcccaaaatctgcgggatttcctgagcaataagagcaagaacaatgtgagtatcttttgttataatactgggtctcttccctcagttcctaaaatcacttcagagccataaaggtgtcttgttattaatgcaagtccctttccacaactactgaatttatgttagtgtaatcacttttcagcttccctggtgactcagttggcaaacaatctgcttgcaatgcaggagaccacctgtaatgcaggagtcctgggtttgttccccaggtcatatcccctggagaaggaaatggaaacccactccagtattcttgcctgggaaatcccatagacagagaagcctggcaggctacagtccataggggtcacaaagaggtggacactatttagcgactgaaccacaatcaccacaatgacttctggaaagcacctaaggatgggtggctaattgccagttgccaggggggacaacctggcagaattgacagatggaacttaagttctagcccatgacttttgggctggggtgagatactagaagttgaatcaattaccaaccaccaataatttaatcaatcaactttatgtaatgaatcctccataaaacccccaaaggatggctttggagcatcccagttgtgagcatagagactcagtcacatggacgactccacacggtccctcaatcccttgctccatgcatctcttcatctgactgtttctgaattacaactttttataataaaacaggattgagctatagaactcacaaaaatgtgtttttatgagttctataggcagcatattaaaaagcagagacatcactttgtcaacaaaggtccgtctagtcaaggctatggtttttccagtggtcatgtatggatgtgagagttggactataaagaaagctgagcaccaaaaaattgatgcttttgaactgtggtgttggagaagactcttgagagtcccttggactgcaaggagatccaaccagtccatcctaaaggagatcagtcctgggtgttcgttggaaggactgatgttaaagctgaaactccaatactttggccacctgatgtggagagctgactcattggaaaagaccctgatgctgggaaagactgtgggcaagaggaaaaggggacgacagaggatgagatagttggatggtatcaccgactcaatggacataggtttgggtggactccaggagttggtgatggacagggaggcctggcatgctgcggttcatggggttgcaaagagtcagacatgactgagcgactgaactgaactgaactgatgagccactctagcaagttaatcacaggaaaggaaggagtcattggaacctccagtctatagcagatcagtcagaagcacagatgacagcctgaacttacaactggcatctgagtcaggaagaggggctatcttatgagactaaatccttaacctgtaggatctgatactatctctgggtagatagtgtcagaattgagttgaattgtaggacttgcaataatgttggaaaattgctcgtggcagggaaaccaccaccactcctagacacacacacacacacacacacacattgggtttgagtgttagaatcattttaaccagtgataagaaagattaccaatggtgcaggaactgccagtggaaacacaaagtctcctgaccaaaacagagcaaatcagaagccaaacctgggttgagaaacaaagaaatgatgacaaaaatcagacagtttgtttcgaaaatctgggaccaggaagaaattaagtgaagagcccaagtgtggcaaaggttcagggtgacactaaactttgttttgcatacctgggaacttcacacatggctagacagaaagagaaaaggacaaattctgtgggaaccaagggggaaataggcacagcaagacgaggacatgggataggtaaccacactcgggacattgtttaactgattcttctggtagactctcatttgcaaggtcctgaggctctcctcccaagcaatttataacctagttaaatacggacagaacagcttaaaatggtgctggatgataccagccagttgctgctggagcaggaggaagacagttgagttttgttggggaaatatggacagcttcacagaagagaggggtgtgaaggaattcggggtgaggaaactgcttgagcagagcttcagccaaggcaattaaagaaggagcaactggcctcgcaggactgaagcccagcggggattcagggagaaaggttgtcagaaacatggtctgggctttttcacacaatctgaaccccagactctggagtctagccactaaggaatcattttatgtgtttaaagaatggagagatatagccgcctgtaatacatctgcaccatgatggtttctctttaaattcacaatcattcatccttagaaaaaatgtaatttctgatgcaatagcttaaattgctgaactgagaaagtcagaggggagaaagggggaggaatgaggtctttgaggacatgaataccctcaatgaatgggcccatcctaaagaggaccctcattattagagaaagtttaaaatactctccattacacccatactctcctccagctaatacctatttctctgttcctcccaggacaaaacttctcagaagaattgtctctactccctcaccttccatttcttcatcaatttactctgtctgttcttatcactctcttgaaactaatcccatcaaggccccagtaacgacctccacgtcaccaaatccagtgagttctttcccacctccattctattttgtctctctatagctgactcctgccttcttgaaaggcacctcctctctcacactcttgatatcctcctacttcactggctgtcacttttcagtctgtcttgctatcttctctttctctaattcaaagtgtgactcagcagatcagcaacatctaggaacccaggagaaatgcagattcttggacttcatctcagaattactaaatcagaatctctaaggtgggcccaaccagtccatcctaaaggagatcagtcctgggtgttcattggaaggactgatgttgaagctgaaactccaatactttggccacctgacgtgaagagctgactcattggaaaagaccctgatgctgggaaagattgagggcaggaggagaaggggacgactgaggataagatggttggatggcatcaccgactcaatgggtaaactccaggagttggtgatggacagggaggcctggggtgctgcagtccatggggtcgcagagtcggacacgactgagcgactgaactgaattgaactgagggtgggcccaggaatctgtgttttatccagaccccaggtgatgcacactgaaagctaagcaccatgatctaaaactgtctgtttcttaccattcagtcctgattcctcttctttcctctggctacactttctttcttgatgttatcatctaagcccacaactttgaaaatcatctaaatactggtggtggtttagtcacctcagtccaactctttgcaaccccagacccctctgtccatgggatttcccaggcaagaatactggttgccatttccttttccaggggatcttcccgaccaagggatcaaacccgggtctactgtaatgcaggcagattcttactgacaatttctaaatgcacatcttcagcaccaaccctcccaatctggagattacattaaaattcaacataccccaaatggaagaaacttctctaaaaatggttgcgtctaaaacttggtcttcctccatttctcaccatttccgaatacatcctctaccaccaagccccatcagtgcccctcaggccccacacaacaatcagaatggtctttgaaatcaggtcaggtcaccaccctgcttaaatcttcaagggcttcctatcacatttaattaaaatctaaagacctggtcattggctgttaggccctacatgacctggtcactttctatgtggcttcctgtattccctttgttgtctaatgtcagaaactataactatctagttcacactaggttctctataaattatttgctgaacaaaatatttcttcttttgaaaataagagaaacatagagtttacttcgttagcttctccacatttgctgaggaggatctatgtgatgttgacaggtaacttcaattgagccaggacacaggagatgcgaagggagactttcaaagaatgtcttcatggtgccataacctcagcacagccaggttccagaggacaaacccccaaacatgcttgtcattcagttcagaatgtagccttccttatatataatgggatatagtagttgtggtgatggtagagactaagatgaggaatgatgtagggccatttgcaaaaggtttctcctgtgggctgaccaacgtgatgttgttcatgaggccagtgaaagcccctaagaatctacaaccccataatggcagttttcaaaaatggccagaaattctttgatactcttcccattgagagatggggtccatgattcctgcccttgaatctgcatgggcatatggctactttggtcaatagcatatagtgaaagtgatgttatgtgatattatgtgacttttgagactatgtgagaagcggcaatgcagcttccatgttgtttactgacagtctcacacttgggtcctcttgggacttcttaagccaggtaagaagctcatcaaacttgagactactatgctgggaggaagccaggccacgtggggaagtcacgtgaaggcacttcaatcagtacacctgattttcaagtcctcccagcccaggtgccagccatgtaagtgactgaactaattccaactcctagctatcacgtcaagcctcagacgtcatggggcagagtcaagtccccattgtgcctgtccaactctttggcctacacaattcatgggcataataaaatggtggtttctttaaaccattaagttttggagtagttgctacatggcaacaatagccagaataggacaaaaggtaatgtcatttcgttccctcaaaccctcacgaactatattgccttttgagcattttcttggttgggggagggaaggaaatcattcagccagttgacattggattcttttgaggaaaaaaggctgagttttggcatcctctaaaggagctgtacattgcccctcctagcaggggaaagtcagtcccttgcccagcctgatctgatcactcactcccagctccacccagctcaagactcaaagagatgcttcactgcccccaatgtgcctcacaataaacaatctctgaggaaagaaggtaaggctctaaaagtagtgtcaacttaatcattatgtaagactgactggatagaaaacagccctggcattgctgcctaatactcatttctagctggccacaatccatttctgctactgaatcatcatcatcccatcgtcatgttttatagacctctcatttctccttcccagcagaactttcaggcccccatccacattcatatattcatcacctcattctacacaattctcctggcctctcccctccctcacttgtccccatcctatttgagagatccaccctgagatatttaccctaatgggtgtctcaatatttttaaacctctttccttgcaagcttagtggagcctcttgcccataacaaggggactagatatttcatttttcccaggtttatacccattgccctggcataattaatattggtactctcaaaagtgcacaaatttgggtaatgatatatatgatccctctaaccctaaaacatgtcttctatcacttgccatccttcacatgagacaaacacctacataaaattttggcagtaataatgatcaagtacacaccatgttttatacaagaaacctcaggtaatgtgcagaatggacttgttaaatggagtgcatttccttcacttatgaatatcataatctaaatcatttattttgtagataatgagcaggaactgagtaaatgacggcaggtgatggctaatatactttctaggcctcaaattttaatctgaaaattcacaaacattgggctcaatccagggcaatagaatttttgtcccttttagaaatttctggttaccaaagttccagaaattgctttctcattccctaatctttcattttctccattacgtaacgagaagctggggctttggccgattttccctttaaagatgatttttatcgtcaacaagcaatttcagggagtgatgagccggggaagcggtattagctgatgctagcgtttaagctagtctcaactcgtttttcccagggacttagattcctgggtctgccagtaaaccccgggcgccggcagctggtgcgcctgagcgtgcgcgcgcgcgccgtcgcctccccgcccctgcccctcctcctccgcccggcgactcacccgccctagttgccagtcgctgacagccgcagagctgagagcgtcttctctctcgcagaagcaggtaaatagccgcgtagtcctttaaactcccagcggaggacgcccaaccctgggtcttgcggccgaggccccagggcacccagccgaatcggattggtgggaggcagaccttgaccgtgagtagggctgggggcttgcggcgggcgcggggaacgtcgggcctgttgagcgtgctcgttggtttttgccagccgccgctcggttttaccctcctggttaggagagctccatttactcggaatgtgggctggctggtccccctcccgaggtatgtgggtggtgtgtaggaatctagccccctcccacgctcgtccactgcgggagtgggatgggcgaatcgcaccggtagaggagccgcaggtccgaggaaccgctggggagctcagaagaacaagggcgaggccccgggatttgggccctcccgaagcccagaggagtcgcggaattgggggtgggggtggtggggaagaaacgggcgcccaacggggcccgacctcggcggtgaggagtgccggagcgtccgtgggcccccagccgctgctgccgaactcctcccgagaggcggccctgcctgccatcacgcggctgggaggtacctgggtagccgcagcgggtgggtctctggcaaccccccggggatcggctctggcgggcgtgcgtggcctgggcttcagcctcggcgcggggaatcatgggccacctggcgctctctccgggccagagaaatccaggtaccgggaacagtgtttcctgggagctctgatgtggtggacccaaaagcaaagcgaaattttccctgtctcgactgatcctccggaaggagggagctcggccgtcgggagactgaggggaggggatcaggcgcctctcggagaaccaccctcatctgccagtgagggtggcaccttcacgcttgatttttttttttcccccttcacacgtttgattattaaacaacgagaagtccgttttttgctgtcctttttccgtttttttttttttttttttccttttggtaccatatgtagcaaatagatttttttaaaatcataagcccaccaccctcaccatctttttttcagtttcctcgtctccagattcttaacaacaaagcagtttcacctccctgatcatggttatccttatctcatggccgggttattttcttgtacttaagagcaatcacgttttattaagcagttccccgaatgctgaacctttgaagtgttacctttccttacaaaagataccacatagaataggattaaaaattttcacaagttgtcagagaaaaataggaacagaaaattgtataaaaatgtcagacctctggaaaatgaacagctctctcagatttgaaaattaacctatgaaaaggaacagttttcctacggaaacattgaggtgctctaacaatgaaaaagaatcagaaaaggaaaaaaacagagttaggatgtgatttgtatatgatttgtatctgatgcaaatttttcatacttgtgaaagaaaaatatcaagattataaaaagataaatggtgaaatgaacaatcatttatgaaataaaatacaaatcaaagcaagtctggatttacaactactagtaaaaacaacagtaacagcaaccacttctggaaagttacctagaaatttgcatattcagtatgtgaggtggcaaggctttggagttagaaatatggctctgcaactaattttacagtttgggacctaatttcctcatcccccttttggacattcataaaatagaggaaattatacctacttcaggagtttgccaagattaactgtgtaaaactgacctttagtgtgtatacttttattcttttcctagtcacactgcactgggggacgttgtgaatctgtatgaaatttgtgaaaaacagtcaggtgatcctttaagccatgaccctaaaaccccactcctgggaacttacctgtaatggaggaaaccaggaaagaagaagaaaagctgcattcacccacagaactcagaatgatctaaaattagatccagtccggagacaacctaaatgtattaataaaatagcagggcagcagctaagaaaatcatagcactttaactgaaaggaacattgtgtaaccatcacgagtcataattttagagcctctctgtgatatacaggaaaaaactgacaggtcaaagtaagattactcagacatggatgcgtttgtggaaaatctgaatgaaaaatgaatccacagtttgctgtgtatgggaggagagttcagtgtcacgtttgctgctttttttaagttagcatcatctcttttttaaaaatactatcatattttttccctgagtagattcattagtggtttaataatttatatactgttattctgttaaataatccgttcttagatttatcaattatagttttttcttttttttttaaggacttctgaatatatttgaaaactgaacagtttcaaccaagccgaagcatctgtcttcccagagacacaaatccaacttgagctgaatcacagcagatgtaggtaccctgcagaatctctttggtcttgtgatggttgaaagtgcccaactgtttcacagaagataagggactgaaaggctgggatcacaaatccttgctgtggaggccactgaaatctatatatgtaacccacacctattatatcactctttcttgtaaaagcgtcttgattttgcagggaaagggacatagctttctctggaatcattctgagttatgtaagaagcagccatttaaaaaatagtataataaaagcaattacctaacatttctgcaccaaatcaacactgaaggtgactatcaacagacaaaaggtttatgaggtaatggtttttctaagctttagttttaatttacctattccattctccctttttagatcttatttccttttccaaggcagccagtttatcaactgtgaactgctgcatatgaagcattcaaaacctgactgtgtctaaagctgtgatggctacagcacaatcatctttgagtgaatagtatgtttaacagttcttacagttgggagaattttttctcagtttgttcatcctttttctcctaaccgtgttctgcttattgctgttctaatattgtgtgatcatgtcaagggaggtgttcccttttatgcaaaacattatgttaaatgttgtcttcccgagaccaagctcggaagattggctaggagtgcagttccgtgggaagccttattataggttcctaaatctcatcactagatactcccaggctgttggcctgatgcagactctagctatgttgcttttcttaaagctcttcacatcactctgaggatggactagactggggaccgtttgcccatttcagtccagggctaggcctcagtgtcagtagaaaaacctccacctcaaaatggtttgtaaatttttgtatagtttgcattagactcttgttaagggacagtgacctcaaaagatgaaaatatgacaaatgagttccacttagcttatgaaaaattggaaatttccccagggcaaggatgggtagagggactgtttggtgccagtttccaatttaaataagtctcaagggtataacatattttgagtatcaaaagtgtggcccctggcacatgaccactggacataagttcctaccagctctgattctcaatccccatgtataaaagggaataagatgaatgggacaatatatggattttgttgttgttgttccttctctctcgttccatcgctctgcctttgtgcttatgcactaatgccacgagattgtatttattatagttttccaatccattctgatacttgccaggccaagtataccttagatgttcttcctgttcagtaatttcttcagtcttcttaattttgagtatcattatattctttaaaatcctctttgagttaggactgaaattgtattgacttaatgattaattggagttgaattggtatctttcaaaatcttcgatcttgattttcccaaccataaaccttgcctgtctttcttttctttccagtcttccagcttttttcatctaagttctactatttattattaggttaaatcttagtttgaattttttgttgccattaatgagtggaatttttttttcacactaaatcttctaataattacaactaattggagtattcactcttttctatatgttgagttcaaaaactgccaccctaataaataagctcattgatttatttgagggcatttttaaatgattgtcctggattttccagatagaaaaatcatacctggctttctccatagcagtctaaaatgcagcaatcacttacattcttgtcttgttaatctcattcacgagaatgcttttcctctttctgttaagatagggtgtggaatatgtattacttctagttctatagaaattttttcaacatcttaaacttaaaaagtcaggaaagcactgattcctgagtttaagtgagaactttgattttaattgaaaactttgcaacatcagagaatcttttttttttctccttagcctactaataggttaattgatttcatgattttgagccattcttgtattcctaaaataatccttattgtcacagtgtattctttcactaaaatatcaaaatcaacttggtggtattttcttttggattttggcacccatattgctaaaggttgggtagctaagagtgtgagcccttcactccctgtggctcttaatactacatctcaggtgaactgcccaaatgtttatctctactgagtaagagttcgagattcttataacaattgcctaaattgatactctcacttgaatggctcatagatatagcaaagttaatatatccaaactatagctttattttttctcacaagcctggccctccattagttttcttgtttgcattaggtggcatcaccaccatctacctagttccaaaagccacaaacctgctcaactctcactctcccatccactctatcagcgtaatcgtttttttctacaaaatacttcccatttttcacagctccgtgctgctttgtcacagttccaggttacaccaccatcttcctttaatcattttctaagtggtctcaccatttcctttcttatccctcaaatttttctctttaacacagcagtaagggtgaactttaaaaaaaaaaaatctgtgatgtgattctcctaagccactttaatggctttgcactgtttcaggctccagacacgtatctccctgtagtcctcaaacccacaaactaaatatcttaccactgtgtcctcagccacagaacagtaatattagaggacatgtaataaatatttgttaagtaatcaatagttattgtacattgtacattgtacatactagctgttccataaatatttatttaatttaaatatttatttaattgaatgaattcaatatcagtttttataattgaatagaaaagtaatccctcccacacagatttttctttctttttttttttttttttttatgaagcaaggaatatgactacttagaaagctggctgccagagaaaatggcagactaatgtcttaaaacaaatatcagtgtctagatgccaggttcttttatagaacagagatggcaggaagatgaggaaataaaggcagaatagagagggagaagtggggtggaagtaaagtaaaaaaggccacgtcctgcaagacatctccagaaaggccagcctgtggaagggatgtgttaatctcttcttgcctgcaaccattcacaggtgggaagtgtcaaattatctccctgtgagctgaacaaaggcacttcagtccaacagttagagagagggactgggttttctgaggcaggctattatatatgattgtaacaacaacagcaacaaaaagcaagtcaaagaaacagttccaatatggagtcagaattggttcttctcagcaacagttccccactgtcaaggtccatttgacaatcttgtaggaaaaggggacagtgatctttctgtttgtcagatgaatctttctgggagtgtctgcctttgatgccagtgttccaaatagtgagatttgttttctttttgttcctccttggaaagtgtctactttatacttgtcaacttagaaatattggacttcaaacccaggagggtttctcaagttaagaaattttgcacttttctgtgtatgggaagatgcaagcgtcagggttcattaaaattatttccttgatgtgtaattcagctgtctggggcctgtgatcctgtattctcaagagtttcctcaaggttcaccatagggagtgggtgcaatctgatgactgctggatggcatgtattctccttcctgagttttctccttcctgagttgacttggggttcaccagctcacattagagggcttcaattgttaattccatttctcaggtcttcccttggtcaggaatttgaccaatatttgggagacatttcatggtcaaactttgcttcacggtgctgtgaggttcatcccaaatcaggcaaaacttcttgatgtaccactctaggtgctaaattttggattagacccccatgaataattaaagattctctggattctaattgtcatccaggagacattttccattgttgcttcttcccatacctagaatcacactattataattattttatgttataaatgtgatctattttctcaagatgtttatccgtagaagttttgttggaggcctgattacacattggttactgcaagagacaactatcttataaattagtcaggatataagtaatgcagctagtaacactgataatgacatagtgcaacaggggtgtgcaaagcacaatctaaaaacaaagaataaagtaaagcaatgattagtataactagttgtagtccagttgcaataatctagtgaccaaaggagccataactgatttaatgatctatcttcagtttcattgtaccagccatgccatagcttaatctttgagacaagcatatgctactggcaggatcaaccagatagaagaagatttaagttttacttttgcttacaaaggatataatttaccaagttactgtaagtcagaggttaaggaagttttccttacacctgaaaaacagatttaaaccagttatttttagatagaaaccataaaaattataacaagttcagttcactcagtcctatgtaactaatcctttttgttaacagctttatgaagccatcaggtttcccattagaattcttcaatgtgttactagttcagcattatggtctaaaagtcagaaacttggatttatctgaaagtcctttttataaatcttcttaaagaggagacatttttacagaggcaacagagtgagaccataactgtccataatgaccaaagacttaagaaggcactttaaatctgattatgatgcaattgacaaagtaacctggttactgctgtgacatacaacagtttcaggtagtagaagtagaatcatgactgataatattctaccaggacatatcacatttttaggaactccatataatctctagtatatcagtatcatttatcatataatttaagatatattattcattggacaacacttgccatgtagtttaacataccaagtgaatctaattagtttaatatctccctttggtgtgtctcaggggccctttgaagcaccccaaagttagctaaagatcaaaggaactttattgtaacttgatttgggaagtcttgtcaaaagagcattaagaaaaaatgttttaaaacacaacaggatcataggtcactgtgaaacaatagttatttacttagccaaaatgacaataaaagatttcaaaagcaaatatagaacagattatttaaaaggtagaaataatctgttatcaaaggagaggaaagccaaatttgttttgtaccaggttactttcaagattcatttagtcaattaaaattttttaaacttagtcctgatcatgtacaaaacactttttcagggtccatgtttcacgaattttccatcacttaatttattttagcacaattttaactttcaagttgttgaatatctggagatatcctaaaatataattatttctgaaagttcactctaagctcttatcttcatttgcattttctttgcttaacagtttattcacatcaagtccctttccttgctgacaaattgtatcaacaaacaaccataaataccaaatataatttaatattaaatatttcccagttcacgtgaacctggagctcatttagcttaattgtatttagaattgtttggtttgtaagcacttacttttatttaaaccaattaaatagagctcttttacaaatcaactgcagcaatattatccaaagacaaagatacatacaaacacacaaacagagaggcctcagttcttatttcaagatttcagtcctgagtcaggcaatgtaaaacccatcagtttacatacgaggttgaattaaaattggatttctcatagatggaataagtcaagctcacttggctagatagctaaatatttgcagaaaaagcacttaggaattctaattatcttggccaacccaacttctaaattactttaccttctctaaaatttgcattttaaaaagacagcataatgagggttcctgagaggacatttgcatctcaaagacatttgcatctcagagttacaggcaagtttttccaaaaatgactttgtttctcctttaatacttacaggcctcttaagataaccagggaaggtctggggagtagtaaaaggatttatgggatttggattatttttggaaccacacttctggtgctgtaaagactacatttgtaaaacacagttttgtttttcttttttgagtgatactgaaggattgacatacccatttacctatgttggaaagttttatctttcctcatttagcttagctcttgggaagacacagaggcaacaatttaggctcctgtaaatcagtctggactgaggggggaacgaggtgaagataagaaagacaggctgaggaatccgcctcatagtcctaccaggaagatagtggccagggcgaatgggtcagtgttttgcttgaaccaatgtgtgcttcacagtgcacagaagctgtcttgctggagttgttgtagccacaagttccggcaaacaaactcactcagaaggacaatgcagatagtggagtgcagtttattacacctgcgggcccaaggcagagtctcctcttagccaaggaccccgaccagtttttgtgaaaaccttatatatcctaagtgtatgtgcccaaacccacctccccgaattccctgaaactagtctgaacaaaggaaaagaaagatacaatcaaagttaacctgtgattcatatgccttaagcctaggtagttaacagtggacagttatcaataggcctgtggtcataccccaataagcataatagaatttatgattctattcggttacacagataattagggtattcttttaggctactgagagtctaggtatgagccctggggctcttgcgggggggggggggggtctggttttccagttagtatgtcatttccatagatactgggcatatagctcaaagtccacagtccagcccaacatggagtcctgctttcaagatggagcctgttctgtctgtttcttcgttcagagtcagatgctctaatagcctggtccattctgttacccacttagcctgtcacaagagacttcaagcgacaggcaccttaatggcttttaagtgcctgacccatgccctcaaaatattaattggcctggtactcagcctaagggagaaggaggtaaggagagccctgacctgttgggaggcagctaccggggcacagagggctattaggcctttagaacaccccagagaataaccctagctagagttccatagctattagtctgtttgcagagggtttaaggagaaagggatgagggagtgtgaggagagtggagaccttaaaggaagtacttctccttttagctcaagcaattagtatcagatgtctatatgttaccaaagtatccggaataaaccaaaatctagccagctagagagtcacattaacatgacttcccagtttcattagacctgtgacctttgtccaaaatgctttataaatggagttttccttcacaggggtgcttcccaagctgaagctgaagctctccactgtcaaagttacccagggctcccgtagggaaattagaatcagatgcctcaagtccaagggagtccccaggcctcttcacttatatcagagtgttcctctcctttgcaaaacacttctaattgcaagagtgtgtaattgtgagccatttaggcccattgctcttctgatcttaacatatctaatatatgtcccccaaagcttttcttcaggatagatgaaatatttcccatttttataagtttcatagcaccaaaacacacacaaaaataggcaaattccaacataaatgacacaaattccagtaccaatacatagatgaaccagtttccagctcaggtaaataaatttaccctacaaaacaaatgaactaatcccaactgtgtgcttgttctgtgcccttcctctggacgcatgcttgctaagccgcttcagttgtgtctgactctttgcagctgtttggactgtgtcccaccatgctcctctgtccatggcattctccaggcaagaatactgccatgcccttggatagactccagtattcttttactccagtatactcattattcttgcctggagaatcccatggtcagaggagactgacaggctacagttcatgaggtcacgaagagtaggacacgactgaaatgacatagcacaaaacaagcacacaagaactagttccccaataaactggttccaactcaggtagaatccaacaacaattcccatctccaacagggtaccccaaccaaattgactagtcctgtaaaggaaaagcccaaatttagaggggaatatgttctcagtgtgcacaccagagcaacttaccctacaaaatcaagtttgtcaactcgtaatagaaaagggcacacaaaaccacaaacaaatgagccagctatcgaatgaagaaaactaatgctatgaaattgggtctgtcaacttgtagaagtttgttgattctttgcttcaactgccaggccctagggccactgataccacttcagggaatcttgaaggagagatcctcagcacaaatggtcccagcagctgctggagccttgccctaatattccctaacagagctggctaaacacaaacagcaagtcaaatttgttaccgaatccaggcttgctctactgagtgaacaacaggccagtgaatcagagatgagatgttgagggaaagaatgtgattttattcagtaagctggctgaccgagaagatggcagactaacatctcaaaataaccatcttcttgggttctggagtgccaggttctttatagaacagaaatgaggggaagtgaggaaataaaggaaaaaggcagaatagagagggagaggcaatgagtcttgggccatcagtcttgcaaaacatctctaggaatccccacacagttttttaattcataaaacttttaactttcacagttaggtctctaatccatttagagcgtgcttttgcatgtagcattaagctccaatttttattttccttcaatttcccagaagtctctgctaaataaactttcctttctcattgatttgttttgtcaatttatcatttatccagtttggactaaagtcaatgtatgtggctttatctctgaacttgttattctgcttcctttgatctatatgttcatttctaggttgttaccatatttattactatgactttatactaggatttaatgtttgataacagtaggttctcacctcatttcctttctaaggttgagtttgttatttgtggacatttcttcatcctttattgaattcctcaaaaaatccagctacaattttgattgtcattattattcatattataagttcatttgtaggaaattgacatctgtataatactagggggttccactgagacttttccatttttacagatcatcttctttgttctttagtagtgttcaatttctttttcccctagtcttactttctcttgaattaagtcaatcctagatactttacagtatgaaagtgaaagtgaaagttgtatctgactctttgtgaccccatggactatacagtccatggaattctctaggccagaatactggagtgggtagcctttccttgctccagggcatcttcccaacccagggatcaaacccaggtctcccacattgcaggcagattctttacgaggtgagccacaagggaagcccaagaatactggagtgggtagcttatcccttctccagtggatcttcctgacccaggaatcaaactggggtctcctgcattgtaggtggtttctttaccaactgagctatcagggaagcccactttacagtatgagttgaaattattactatcttatttattaatttttttctagtcaattattgctgatatagagaaatgctgttgattttttaaaaccaatccatagccttgctgaactctagttgagtttcctgttacctctgccaagtgtgtggaattttctatgtatatgatcacattaattccaaataatgacagctggagctcttttcttacagttattgcaccagtctttgtccttgcatatggcattggaagagggcttcccaagtggctcaatgttaaagaatccacctatcaatgaggagatccaggtttgattcctgggtcaggaagatccctttgagaaggaaatggccacccactccgttgttcttgcctggggaatctaatggacagaggagcctggagaactacagtccatggggtcacaaaagagtcggacacaatctagcaactaaaataacaataatggcactggaaggatctccagtcctgtgacaggggacgtccttgttttgtttctgatcataaagggactgcattcaaaaattatctattaattatgtttaccatttctgttatataatctttattaagttaagcaggtttcctcctattcctagtctgctaagagtatttttcttagtgataggtattcagttcagttcagtctgttgtgtcccactctttgtgtcctccatggactgcagcatgccaggcttccctgtctatcaccaactcccagagcttactcaaactcatgtccattgagtcagtgatgccatccagtcatctcatcctctgtcatccccttctcctgccctcaatctttcccagcatcagggtcttttccagtgagttcttcacatcaggtggccaaagtgttggagtttcagcttcagcatcagtccttccaatgaatattcaggactgatctcctttaggatggactggttggatctccctgcagtccaagggactctcaagagtcttctccaacataacagttcaaaatctaggttggtcataactttccttccaaggagtaagcgtcttttaatttcatggctgcaatcaccatctgcagtgatattggagccccaaaaataaagataggtattgactattatcaaatacttagtatcttgatgtgctaaaggattaggcagcaacctgactattctctgagaacatcttatatgaagtgttataacacagccaggcattcagaaacctaatgtgcatttctagtacttttactgtaatcacagatatgtttcttgaatttgctaatctttgtagcataatttgtacagtgagagattttggattaaataatacaagatcttctttattttatcacaaacagaaagaaaattctccaaggtctcattaagttttgagttcttcttatttttagaaatgattctctacttcaaaaaaattttttatgattttcataattaagtgttgcttttgtgttctcaactgattttaaaatgattttgtcttaatatagtttacaattatccaactttattctaatattcaattttaaagtaatgatcagcaacatatgcccgtggccttatgtcctattgcctgtatctccagtccatggtcattcctcacctccagactcaccaagcttaccagactgactggtatctcttgtcccacgtgtagcttgataatggcattgtcaaagagacactcctcatttgtcttcctatggtcttgctccaccttccgtcttctgtccacagaattgtccaatccagaaatctgggtgccacctcaattcttctagtctttcatcttccatgtcaaacaccaagccttccagaaagttctcatgttcaattccctgaggtgtttgagcagaattgtgagcagcatggaagccaagatctgagccccatgagcaaatggagagatcagggaagcaggcctgcaaaagaccaggtgcaagaatgaggccatttggcatatcccaggaccctgttcctctggcctttacccaaaacagactccaaaaattctagtagacatagtctgagcagtttaactggccttataactttcaaatatatttttatttatacccaggcatttcaataatgatagtagaaacataaatgggatgttaattcattgtttagtcatccttcctctgaatattatccaagttagtctttagttctgaaggtcatgaaaaataattttataatatttggtgccacttttatttgaagatgtcccagtgctggggatgactaatgtcagcattacaacatatgccatttttggttttatggcaaatggtattttggaacatgtagtttgatgtggggtacagtagaaagtgtttaatgatcattctactgtgcatctttaatttctgcccttggaaccacccagggtaagtgagatattcattctgaaagatctgaatcttcaattcattcatctataatttgatgaatgtacattcacaaaggttcataggttatcatgcaggatactttgttcccaaactgtgcttgcccttacatgtaagatatgtgtcttttgtaccaaaaattaagagaaaataagtcacttatgaaccattaaatgctgaactaagactcattcagtgagtgagtaactgcaaatactatgaacacagcctttcttacccctttttgaatagccccattgtctgtctatagaaagaaaaattactttataggtgtgtttgcaaaatcttgcctgtttcctgtttccaaaagttattgtattgagaattcctttgagaaaattcttgttgggatttatgtgttcagaagatgataattccttcatttaacagatatctattgtgtaccttctctgtgccaggctctgccctggcccgctaagaagatagcagcaaacaaaagaggctcattccctgcttacattcctacatgaggaaagaggacatgaaccagctattcagaaaagtatttaatgatctcagcacctaccttggggtcttcccaactggacattagaatcacttccatagggcccatgccagggttcagaaggttccaggaactaatatcccttataacaacccaataggcagagtttctagggtccccacaagaacaagcccagttgcaagaatcactactttaaagaagttcaaagctatggtaaacctaccagatgtttatagtttcttccaatttatgatacagtgtaccagtcagaggttatttttatcataagcaatgttgctggcattctacatttatcaagttactaggaaacagagccaggaattattttaaggtcaactttgtccttagagaaggaagagttgtgttaacactttacctataattactttcgtgagatgtatggaatgtgaagaatatttatgacctagactgtttatagctgatgccactgctatgcagtcattatgctacagactttaagtgatttttacatgggcatatgatgctgacaccctctttattttgcagataagtcatcatggtgaaaagccacataggcagttggatcctggttctctttgtggccatgtggagtgacgtgggcctctgcaagaagcgaccaaaacctggaggaggatggaacactggggggagccgatacccaggacagggcagtcctggaggcaaccgttatccacctcagggagggggtggctggggtcagccccatggaggtggctggggccagcctcatggaggtggctggggccagcctcatggaggtggctggggtcagccccatggtggtggctggggacagccacatggtggtggaggctggggtcaaggtggtacccacggtcaatggaacaaacccagtaagccaaaaaccaacatgaagcatgtggcaggagctgctgcagctggagcagtggtagggggccttggtggctacatgctgggaagtgccatgagcaggcctcttatacattttggcagtgactatgaggaccgttactatcgtgaaaacatgcaccgttaccccaaccaagtgtactacaggccagtggatcagtatagtaaccagaacaactttgtgcatgactgtgtcaacatcacagtcaaggaacacacagtcaccaccaccaccaagggggagaacttcaccgaaactgacatcaagatgatggagcgagtggtggagcaaatgtgcattacccagtaccagagagaatcccaggcttattaccaacgaggggcaagtgtgatcctcttctcttcccctcctgtgatcctcctcatctctttcctcatttttctcatagtaggataggggcaaccttcctgttttcattatcttcttaatctttaccaggttgggggagggagtatctacctgcagccccgtagtggtggtgtctcatttcttgcttctctctttgttacctgtatgctaatacccttggcgcttatagcactgggaaatgaagagcagacatgagatgctgtttattcaagtcccgttagctcagtatgctaatgccccatcttagcagtgattttgtagcaattttctcatttgtttcaagaacacgtgactacatttcccttttggaatagcatttctgccaagtctggaaggaggccacataatattcattcaaaaaaacaaaccggaaatccttagttcatagacccagggtccacctggttgagagcttgtgtcctgtgtctgcagagaactataaaggatattctgcattttgcaggttacatttgcaggtaacacagccagctattgcatcaagaatggatattcatgcaacctttgacttacgggtagaggacattttcacaaggaatgaacataatacgaaaggcttctgagactaaaaaattccaacatatgggagaggtgcccttggtggcagccttccattttgtatgtttaaagcaccttcaagtggtattcctttctttagtaacaaagtatagataattaagttaccttaatttaattaaactaccttctagacactgagagcaaatctgttgtttatctggaacccaggatgattttgacattgtttagagatgtgagagttgaactgtaaagaaagctgagtgctgaagaattgatgcttttgaactctagtgttggagaaaacttgagagtcccttggactgcaaggagatcaaattagtccatcctaaaggagatcagtcctgaatattcattggaaggactgatgctgaagctgaaactccaatactttggccacctgatgggaagaactgaaggcaggaggagaaggggatgacagaggatgagatggctggatggcatcatggattcaatggacatgagcttgagtaaactccaggagttggcaatcgacggagtcctggcatcctgcagtccatggtgtcgcagagttggacacgactgagtgactgaactgaggtgaacccagattttaacatagagaatgcagatataaaaactccatattcatttgattgaatcttttccttaaccagtgctagtgttggactggtaagattataacaacaaatataggttatgtgatgaagagaatagtgtacaaagaaaagaaatatgtgcatttctttattgctatcataattgtcaaaaaacaaaattaggtccttggtttctgtaaaattaacttttgaatcaacagggaggcatttaaagaaatatcttaaattagagacagtagaaatctgatacattcagagtggaaaaagaaattctattacgattatttaagaaggtaaaattatttcctgggttgttcagtattgtcacctagcagatagacactattgttctgcactgttattactggcttgcactttgtggtatcctatgtaaaaatacatatattgcatatgacagacttaagaatttctgttagagcaattaacatctgaactatctaatgcattacctgtttttgtaaggtactttttgtaaggtactaaggagacgtgggtttaatccctaggtcatgtaaatcccctggaggaggaaatagcaacccactccagtattcttgccaggagaatcccatgggcagaggagcctggcagggtgcagtccatgcatagggttgcaaagagtcagacaagacttgagctactaaacaataacaacaataaatgctgggttggctaaaaggttcattaggttttttttctgtaagatggctgtctttaacttcattcgaaacaattttgttagattgtatgtgacagctcttgtatcagcatgcatttgaaaaagaaaacaacttaccaaaattggtgaatttttgtatagccattttactattgaagatggaagaaaagaagcaaaattttcagcatatcatgctgtattatttcaagaaagataacacaaccaaaatgcgaaaatgtatttgtgcagtgtatggagaaggtgctgcaactgatcaagcttgtcaaagtagtttgtgaagttttgtgctggagatttcttactggacaatgctccacagtcgggtataccagttgaagttgatagtgatcaaattgagatattgagaacaatcaatgttataccacgtgggagatagctgacatactcaaaatatccaaatagaaccttgaaaaccatttgcaccatctcagttatgttaataactttgatgtttgagttccacataaattaagcaaaaaaaaaacaaaaacaaaaacacacaaccttgaccatatttgcatatgcagttctctactgaaatgaatgaaaacacttttgtttttaaaaacagattttgatgaacagtggatactatacaataacgtagaatggaaaagactgtggggtgagcaaaatgaaccagcaccaccaaaggccaggcttcatccaaagaagatgtgtgtatggtgggattggaaagtaatcctctattatgggattcttctggaaaaccaaaaaatcaattccaacaagtactgctcctaattagaccaactgaaagcagcattcaatgaaaagcatccagaattagtcaatagaaagcatataatcttccatcaggataacacaagactacatttctttgatgacccagcatggctgagaggttctgattcacctgctgtattcagacattgcatctttggatttccatttatttcagtctacagaattatcatcatgaaaaaaatttccattccctggaagattgtaaagtgcatctggaaaacttctttgctcaaaaagataaaaagttttgtgaacacagaattatgaagttgcctgaaaaacggcagaagatagtgactatgttgttcagtaaagttcttggtgcaaatgtgtcttttatttttatttaaacactaaaggcacgttttggccaacccaatactgaatacttaaaggaaactcttccgtgttgtccttagccttacagcgtgcactgaatagttttgtataagaatccagagtgatatttgaaatacgcatgtgcttatattttctatatttgtaactttgcatgtacttgttttgtgttaaaagtttataaatatttaatatctgactaaaattaaacaggagctaaaaggagtatcttccacggagtgtctggctgttttcaccagtgtgcacaccatgttggcagcttcatttggggggttaatatgagaaaagtggcacattcagtcctcacactgccagttgcggcaggagggcttctcctgatcctgcctcagccttactcccagtcacatgccagctgttctctgctaccttttcatatttttccatgaatacccgtcaaagttactactatagcggaggaaaacagtccttgcattctggaagattttttctgaccaggattttgaaatagaggattttcgtgattaagatgagacttaacaaagtatctaccttatgcctgtacccacccttgacaccatttcaggtcataaactgtgaggcctggtgacaacacccattgaattgaaattcaacactgtacggtcaatatggctactttcctttgttacaggctttcaaatggttcttcatatgtttcctccttcccaagtatgaggtgccagctcccagttttccttcacaaaggttttcttctgcaactgtagttcattaacagccggaagaaataataaatgatagtggttgaaatcataacatttattaacactttaataaatgccagtgtccttcagtatctgaacagaggatcaactttgcattaaaaatgaaaagattaaaaatcaacatcttgatatcccataattcacaaaataatttaaaaatgacataaaatcctcaaaagcattactcagttaatctttaacataagaagtgctaggactattttcatgctgtccttttggccatatgtaagattatttaaaaatagactattcattatctgccaatcataatctcccaagaataccccactgaaaagatgtcagttatacaaagcaaggtatttacagggccgaagtgaatgatacacatctgtatttttctcaggctaccatgttttcttcctgttacttccaattcctttgagttgtgctaaagaaatttctttatatttcatatgtatttttaaatagaggataattactttacaatattgtgatggtttctgccatacatcagcatgaatcagcatagggcttcccaggtggcactagcggtaaagaactcacctgccagtgcaggagacataagagatgtgggttcaatccctgagtcaggaagatcccctggaagagggcatggcaacccactccagtattcttgcctcgagaatctccatgggcagagcagcccggtgggccacagtccataaggttgcaaagagtcggacacaactgatgtgacttagcatgcatgcatacatatggccccttccctcttgaaccccctctaccacctccctccccacccacccctctaggttgtcgcagagtactagctttggtttccctgcatcatacattgaactctcactggctggctgttttacatatggtatatgtttcagtgctattctctcatatcatctcacactctccttcccttactgtgtccaaaatgtctgtgtttcctttgctgccctgcaagtaggactatctttctagattccatatatatgtgttaatgtatgatatttgtctttctctttctaacttatttcactctgtataataggctctaggttcatccacctcattagaacagactcaaatatgttcctttttatggctgagtaatattccattgtgtatatgtaccacaacttcattatccattcatctgtctatggttggacatctaggttgtttccatgtcctaggtattgtaaattgtgctgcaataaacattgaggtatatacatctttttcagttctggtttcctcagggtatatgcccagtagtgagactgctgggtcatatggtaactttgggcttcccttgtggctctgctggtaaagaatccacctgcaatgcgggagacctgggtttggtcctgggctgggaagaccccctggagaagggaatggctacccaccccagtattctggcctctagaattccatggactgtatagtccatggagttgcaaagagttgcacacgactgagcaactttcactcacctatggtaactttatttctagtcttttaaggaaactccatactgttctccatggtggctgtatcagtttgcattatgaccaacagtgtcagagagttcccttttctccacatcctctccagcatttattttttgtaaactttctgatgatggccattctgaccaatatgagatgacatctcattgtagttttgtttgcatttctctaaaatgagtgatgttgagtatcttttcatgtaattattagtcatctgtcatctttggagaaatgtctgtttgagtcttctgcccattttttaaatttggttgttttttgttactgagctgcttatgtattttggagattaattcctttcagttgtttcatttgctattattttctcccattctgagagttgtcttttcaccttgcttatggtttccttcattgtgaaaaaacttttaagtttaattaggtcccacttatttatttttgtttgtatttccattattctaggaagggggtcaaagaggatcttactattctgcctatgttttcctctaagagtcttatagtttctgatcttacatttaggtctttcatccattttgagtttatctttgtgtatggtgttaggaagtgttctaatttcattcttttacatgtagctgaccagttttcccagtaccagttattgaagaggctgtcttttctccattgtatatttttgcctcttttgtcaaagataaggtcctcatcagatcagatcagatcagatcagtcactcagtcatgtctgactctttgcgaccccatgaatcgcagcatgccaggcctccctgtccaacaccaactcccggagtttactgagactcacgtccatcgagtcactgatgccatccagccacctcatcctctgtcatccccttttcctcctgcccccaatccctcccagcatcagagtcttttccaatgagtcaactctttgcatgaggtggccaaaatattggagtttcagctttagcatcattccttccaaagaaatcccagggctgatgtccttcagaatggactggttggatctccttgcagtcggactctcaagagttctccaacaccacagttcaaaagcatcaattcttcagtgctcagccttcttcacagtccaactctcacatccatacatcaccacaggaaaaaccatagccttgactagatggaccttggttggcaatgtctctgcttttgaatatgctatctaggttggtcataactttccttccaaggagtaagcatcttttaatttcatggctgcagtcaccatctgcagtgattttggagcccagaaaaataaagtctgacactttccactgtttccccatctatttcccatgaagtaatgggaccggatgccatgatctttgttttctaaatgttgagctttaagccaactttttcactctccactttcactttcatcaagaggctttggtgcatggatttatctccaggctttctattttgttccattggtctatatttccatttctgtgacagtaccatactgtcttgatgaccatagctttgtagtatagtctgaagtcaggaaggttgattcctccagtgtcattcttctttctcaagattgctttggctatttggggtcttttgtgtttccatacaaattgtgaaagtatttgttctagttctgtgacaaataccattattagtttgataggaattgcattgaatctatagattgctttggataacatagtcattttcactatattgattcttccgatccaagaacatggtatatctctgagacaggaaacccgcgctgtgagtgcttgatcaagcccaagagaatagtccgcaagccggtttttgtgtgtttgtttttggccctttggtaactatcggtaaatttattcctaggtatttttgttgttgttgttgcaatggtgaatgggattgtttccataatttctctttctgatttttcattgttagtttataggaatgcaagggatttctgtgtattaattttatatcctgtgactttactgtattcattgattagctctagtaatttttatgtggcctctttatagagtttcctatatagaggatcacatgatctgcaaacagagttttactacttcttttccaatctggatccttgtgttaaaggatttttactaaaaaattaaaatatcaattttaaataactgagtctaactcttacagaaggtttttctggagaagtgtcaggtgtcaaactttctttcccttcctctctctctggaattaaagccaaagaagtgtcctctgccttggaagaaattttgggtctgtattgcttctcactctagtgggaaccttaatatggccagaacctgagcttccccaggctcaggccctgaccttccattggtctaagcaactgacctacatagttcatttccacttgagaatggtcagttcctctctggctctttgaaactcctggaggatttagcttctcctgcattaactggaggaactaaacccatcctttgccccactcctgtgaggcctacccctgttctccaagaagccacaccttctgctacacacattcagcctatgagcttcaactctgccttgctacaattttcctttcctggagagctggtgttctgttctttccctggagtagtgtgcctcaaacttgaatgtgcacctgacaggggcccaagattctgcatttcttacaggttcccagatgatgccatgctggttctgtgaaacttcactggaacagctccctcaggatttcacactggagcctctaccagcaccacctgaagttcaacacaagttgctgcaccccaccccagagtttctgattccagagtgcagggtaggaccagagaatttacatttctaacacactccctggcaatgctgctgttgatgtggagattgcaaatggagctccactgctctacaggaagatgtacatggaatagaaggcaacctggccctgaaaaatagagcagttaggagactaaaaatctaattggaatgctccctgaggaggagagagctgagagctctagggatgaaaagcaaaggagacataaggaagtagttaatacctgctgcctgaaaaactggaagcactggtgagtcctgaggcccaccactagtgagagattcagctaaacttggaatagtagccaggccacaaatgcagcacttctcaaattcagatgtgcgcacaaatcacccaagaaccctgtcaaaatgcagttctgaggccatatgtttgatgtaagcttggagatgtgtcatttctataagctcccaggtgatgtgtggtcccagtggtcccaggaccacaccaagaaacaaggacctagaagcctaagtcatctcttctaaccgtggccaagactttaaataagcattgaagtctcaggagctggggggaggtggggagtagccaatagagagtcttcaccttttcttgatttagccctaagttttgcctgtcgtgctttgagagcacattcctcttacctatcaacctcctgctggcagcagtgaagtcagcttgtgtattatctctgaaacaagctgaattagttggctgcccatgggaaatatcaaatccagagacactctgtcagtttttcaaggtcatacaaatagtgagtgaaaattttagttgctcagtcatgtctgattctttgcaaacttatggactatagctgccaggctcctctgtccatggaattctccaggcaagaatactggagtgggttgccataccctcctccaggggatcttctggacccggggatcttctggacccagggatcaaaccctctctctgttgcaggcagattctttactgtctgagccaccagggaagcccacacaaatagtatgttcaccaaagcacattgtggaaactctttgccttggtttgtgtttatatttaagggtttggctcaaaggtccgacatctcagtcactgtgcacaactcatggcctctgtcaagggtgccccctggtgcagggctccagcttgaggggactcagttgaatccaaggggaacctgaaggaagggtcagaaatcctaaaagcaaattcagcccaaaatgcctcctaccctatttgattcctccatcactcactgtcccatacacacttctctcatattatttcagaagtgacctgtagccagggcccatagattagtagccctctccaatcaaaccatagttccctaagccctagaacacatacatgtcacctcgtgccagagcccctaggctggaggccaccagggtaattgggactgggggcttctttctccctaactgtcctggcaaatctgcccctttcctccttctctaaaaacaaacagtaaacaaacaaaagcaagatcgttatcttaatctttatatcgagtaaaaataaaagttttcagtaactctattctttagcacccttactcaacctaatcatttaagaaaaccttacaggcccttgtttcattgcctttcttgttgaatataccatcttgattagttttctggggttgccattaaaaaaaaaaaaaagtgccacagattgaatggcttaaacaacagatatttactttctcactcttctggagactggaagtctgagattaaagtatcatcagggttggtttcttctctgacttgtagatggcctccctatgtcttcccacagtcttccctttttgtgtctctgtgtcctaatctctttttataaggaccagttcatgctctatcatgagaccctatggactatagccctctaggctcctctgtccatggggttttccagacaagaatactgtgggttgctattttctcctctaggcaatctttctgacccagggatcaagcccacgtgtcctgtatctcctgcattgcagatggattctttactgctgagccactggggaagcccttttataaggactgggcttcccttgtggctcggccggtaaagaatacacgtgcaatgcaggagaccagggtttgatccctgggtcaggaagatcccctggagaagggaatggtaacccactcctcaaattgtatttgaggtgaggactgctactgctgctgctaagtcgctttagtcgtgtccaactctgtgcgaccccatagacggcagcccatcaggctctcctgtccctgggattctccaggcaagaacactggagtgggttgccatttccttctccactgcatgaaagtgaaaagtgaaagtgaagtcaccactcctcaaattggatttgaggtaaggacaccactcctcaaattgaattagggctcaccctaatggcttcatcttaacctaactttaactctttaaaggccctaactccaaatacagtcattttgaggtattaaggactatgactccaacacctatcaagaaatgtcacagcagtatgttagtgtcagtctcaagagcgctcaaaggcagtcccaggactaagacaaccttaatggcagcctcacagtcacattctattcccttatcaggatcacactattccttcaatagactgagccactgcccatcaatccacttagaattgccaagggtacctatctcatagtgcccattgcagagcaaacagaaatgcttccattctggatacagaccctgaaacccagccaccatgcccccatggctcacacaaagagcttcataatcaaacaaatttgccctttggtttgtatccacacacaaatacactacaaacacacctggcttagagttacactgattatgagttaattgacataaaactgagtgttagctataatttaaggggtactatctctaattttcttgaagagatctctcatctttcccattctgttgttttcctctatttctttgcattggtccctgagaaaggctttcttatctcttcttgctattcttgggaactctgcattcagatgcttatatctttccttttctcctttgcttttcgcttctcttcttttcacagctatttgtaaggcctccccagacagccattttgcttttttgcatttcacgcaaagatgggcttgataaaggacagaaatggtatggacctaacagaagcaggagatattaagaagaggtggcaagaatacacagaagaactgtacaaaaaagatcttcatgacccagaaaatcacgatgatgtgatcactgacctagagccagacatcctggaatgtgaagtcaagtgggccttaggaagcatcactaccaacaaagctagtggaggtgatggaattc

SEQ ID NO: 2 - protein, 256 aa, Bos taurus
MVKSHIGSWILVLFVAMWSDVGLCKKRPKPGGGWNTGGSRYPGQGSPGGNRYPPQGGGGWGQPHGGGWGQPHGGGWGQPHGGGWGQPHGGGGWGQGGTHGQWNKPSKPKTNMKHVAGAAAAGAVVGGLGGYMLGSAMSRPLIHFGSDYEDRYYRENMHRYPNQVYYRPVDQYSNQNNFVHDCVNITVKEHTVTTTTKGENFTETDIKMMERVVEQMCITQYQRESQAYYQRGASVILFSSPPVILLISFLIFLIVG

SEQ ID NO: 3 - DNA, 31412 bp, Ovis aries
gatccacccagtccatcctaaaggagatcagtcctgggtgttcattggaaggactgatgttgaagctgaaactccaatactttggccacctgatgcgaagagctgactcattggaaaagaccctgatgctgggagggattgagggcaggaggagaaggggacgacagaggatgagatggttggatggcatcactgactcaatggacatgggtttgggtggactccgggagttggtgatggacaggaaggcctggcatgctgcagttcatggggtcacaaagagttggacatgactgagctactgaactgaactgaactgatgagccactctagcaagttaatcacaggaaaggaaggagtcattggaacctccagtctatagcagatcagtcagaagcacagatgacagcctggacttacaactggcatctgagtcaggaagaggggctatcttatgagactaaacccttaacctgtaggatctgatactatctctgggtagatagtgtcagaattgagttgaattgtaggacatgcagtaatgttggaaaattgctggtggcagggaaaccaccaccactcctacaaacacacacacacacacacacacacattgggtttgagtgttagaatgattttaaccagtgataagaaagattaccaacagtgcaggaactgccagtggaaacacaaagtctccgtagtccagaacagagcaaatcagaagccaaacctgggttgagaaacaaagaaatgatgacaaaaatcagaccgtttgtttcaaaaatctgggaccaggaagaaattaagtgaagagcccaagtgtcgcaaaggttcggggtgacactaaacattctttgcatagctgggaacttcacatatggctagacagaaggagaacaggaaaaattctgtgggaaccaaggggaaaacaagcacaagaacaagatgagaacgtgggataggtaaccacactcaggacattgtttaactgattcttctagtagactctcatttgcaaggtctgaggctctgctgctgctgctaagtcgcttcagttgtgtctgactctgagcgaccccatagacggcagcccaccaggctcccccgtccctgggattctccaggcgagagtactggagtgggttgccatttcctttgaggctctactcccaagcaatttataatctagttaaatatgggcagagcagcttaaaatggcgctggatgatacaagccagttgctgctggagcaggaagaagatggttgagttttgttggggaaatatggacaacttcacagaagagaggagtgtgaaggaattcggggctgaggaaactgctggagcagagcctcagccaaggcaattaaagaaggggcaactggcctagcaggactgaagcccagggggtactcagggagagaggttgtcagaaacacggtctgggctttttcacacaatctgaaccccagaccctggagtctagccactaaggaatcattttatgtgtttaaagagtggagagatatagccgcctgtaatacgtctgcagcatgatggtttctctttgaattcacaatcattcatccttagaaaaatgtaatttctgatgcaataccttaaattgctgaactgagaaagtcagaggggagaaagggggaggaaggaggtctttgaggacatgaatacccccaatgaatgggcccatcctaaagaggaccctcactactagagaaagtttaaaatactttccattacacccatactctcctccagctaatgcctatttctctgctcctcccaggacaaaacttctcagaagaattgtctctactccctcaccttccatttcttcatcaatttactctgtctgttcttatcactctcttgaaactaatcccatcaaggccccagtaatgacctccatcaccaaatccagtgagttctttcccacctctactctatgtggtctctctaaagctgacccctgccttcttgaaaggcacctcctctctcgcgctcttgatatcctcctccttcactagctgtcacttttcagtctctcttgctatcttctctttctttaatgcaaagtgtggctcagcagatcagtaccatctaggaacccaggagaaatgcaggttcttggacttcatctcagaattactaaatcagaatctctaaggtgggcccaaccagtccatcctaaaggagatcagtcctgggtgttcattggaaggactgatgttgaagctgaaactccaatattttggccacctgatacaaagagctgactcattggaaaagaccctgatgctgggaaagattgagggcaggaggagaaggggacgactgaggatgagatggttggatggcatcaccaagtcaatgggtaaactctgggagttggtgatggacagggaggcctgctgtgctgcagtccatggggtcgcagagtcagacacgactgagcgactgaactgaattgaactgaaggtgggcccaggaatctgtgttttatccagaccccaggtgatgcacactgaaagctaggaaccatgatctaaaactgtctgtttcttaccattcagtcctgattcctcttctttcctctggctacactttctcccttgatgttatcatccaagcccacaactttgaaaatcatctaaatactggtggtggtttagtcacctccgtcatgtccaactctttgcaagcccaggctcctctgtccatgggatttcccaggcaagaatactggttgccatttccttttccaggggatcttgctgaccaagggatcaaacccgggtctcctgtaatgcaggcagattcttactgacaatttctaaatgcacatcctcagcaccaaccctcccaatctggaggttacattaaaattcaacataccccaaatggaagaaacttctctaaaacttgttgcctctaaaacttggtctttctccgtttctcaccatttcagaatacctcctctaccaccaagccccaccgttgcccctcaggccccacacaacaatcagaatggtctttggaatcaggtcatgtcaccaccctgctgaaatcttcaagggcttcctatcgcatttaattaaaatctaaagacctggtcattggctgttaggccctgcatgacctggtcactttctatgtggcttcctgtattccctttgttgtctaatgtcagaaactatatctaattcacactagggtctctataaattatttgatgaacgaaatttttcttcttttgaaaataagagaaacatagagtttgacttctttagcttctccacgtttgctgaggaggatctatgtgatgttgacaggtaacttcagttacgccaggatgtgggagatgagaagggagactttcaaagaatgtcttcatggtgccataacctcagcacagccaggccccagaggacaaaccctcaaacatgcttgtcattcagttcagaaagtggccttccttatatataatgggatatagtagttgtggtgatggtagagactgagatgaggaacgatgtagggccatttgcaaaagctttctcctgtaggctgaccaatgtgatcctgttcttgaggccaaggacagcccctaagaatctacaaccccataatggcagttttcaaaaattgccacaaattctttgatactcttcccattgagagatggggtccatgatccctgcccttgaatctgaatgggcatatagctggctactttggtcaatagcatagagtgaaagtgatgttatgtgacttttgagactatgtgagaagtggcaatgtagcttccatgttgtttactgacagtctcccacttgggtcctcttgggacttcttaagccaggtaagaagctcatcaaacttgagactgctatgctgggaggaagccaggccacatggggaagtcatgtgaaggcacttcaatcagtacacctgattttcaagtcctcccagcccaggtgccagccaagcaagtgactgaactgattccaattcctagctatcacgtcaacctcagacactgagtcttcccaacagcagcctcatatgtcatggggcagagtcaagtccccattgtgcctgtccaactctttggcctacacaattcatgggcataataaaatggtggtttctttagaccattaagttttggagtagttgctacatggcaacaatatccagaataggaccaaaggcaatgtcatttggttccctcaaaccctcacaaactatattaccttctgagcattttcttggttgggggagggaaggaaagcattcagccagttgacattggattcttttgaggaaaaaaggctgagtttcggcatcttctaaacgagctgtacattgccccttctggcaggggaaagtcaatcccttgcccagcctgatctggtcactcactcccagctccacccagctcaagactcaaagagatgcttcactgcccccaatgcacctcacaataaacaatctctgaggaaagaaggtaaggatctaaaagtagtgtcaacttaatcattatgtaagactgactggatagaaaacagccctggcattgctgcctaatactcatttctaactggccacaatccatttctgctactgaatcatcttcatcccatcgtcatgttttatagacctctcatttctccttcccagcagaactttcaggcccccatccacattatatatacatcacctcattctacacaattcttctggcctctcccctccctcacttgtccccatcctatttgagagatccaccctgagatgtttaacctaatgggtgtctcaatatttttaaacctctttccttgcaagcttagtggagcctcttgcccataacaagggggctagatatctcatttttcccaggtttatacccattgccccggcataattaatattggtactctcaaaagtgcccaaatttggataatgatgtatatgatccctctaaccctaacatatgtcttctaacacttgccatccttcacatgagacaaacccctacataaaattttggcagtaataatgatcaaaaacacaccatgttttatacaagaaatctcaggtaatgtgcagaatggacttgttaaatggagtgcatttccctcacttatgaatatcataatctaaatcatttactttgtaaataatgagcaggaactgagtaaatgacggcaggtgatggctaatatcctttctaggcctcaaattttaatctgaaaattcacaaacattgggcttaatccagggtagtagaatttttgtccttttcagaaatttctggttaccagagttcccgaaattgctttctcattccctaatctttcattttctccattacgtaacgagaagctggggctttggccgattttccctctaaagatgatttttatcgtcaacaagcaatttcagggagtgatgagccagggaggcggtgttagttgatgctagcgtttatgctagtctcaactcgtttttcccagggacttagattcctgggtctgccggtaaaccccgggcgcccgcagcgggcgcgcctgagcgtgcgcgcgccgtcgcctccccccccccgcagctcctcctctgcacggcgactcaccagccctagttgccagtcgctgacagccgcagagctgagagcgtcttctctcccagaggcaggtaaatagccacgtagtcctttaaacccccagcggaggccgcccccggcttgcggccgaggccctagggcactcagccggatcggactggctgggaggcagaccttgaccgtgaggaggactgggggcttccggcgggcgcggggaacgtcgggcctgtttagcgtgctcgttggtttttgccagccaccgctcggttttgccctcctggttaggagagctccatttactcggaatgtgggcgggggccgcggctggctggtccccctcctgaagtatgtgggtggtgtgtaggaatctagccccctcccacgctcgtccactgcgggagtggcatgggcggatcgcaccggtagaggggccgcagtccgaggaaccgctggggagatcagaagaacaagcgagaggccccgggctctgggccctcccgaagcccagcggagacgcggaattgggggtggggggtggggaagaagcgggcgcccaacggggccagacctcggccgtgaggagtgccggagcgaccgtgggcccccagccgctgctgccgaactcctcccgagaggcggccctgcttgccatcacgcggctgggaggtacctgggtagccgcagcgggtgggtctctggcagccccctggggatcggctcgggcgggcgtgcgtggcctgggcttcagcctcggcgaggggagtcatgggcgacccggccctctctccagagaaatccaggtaccgggagcagtgtttcctgggagctctgatgtggtcgacccaaaagcaaagcgatattttcgctgtctcgactgaaggagggaactcggccctcaggagactgaggggaggggatcaggcgcctcttggagaaccaccctcatctgccagtaagggtggcaccttcacgtttttttttttgttgttgttgttttctcacacgtttgattattaaacaacgaggagaagtccgttttttgctgttctttttcgttttttcccccctctcttttcttttggtaccatatgtagcaaatagattttttaaaatcataagaccaccatcctcaccatcttgtttttcagtttcctcgtctccagattcttaacaaagcagtttcacttccctgatgatggttatcctcatctcatggccaggttattttcttgtacttaagagcaatcactgtttattaagcagtttcccgaatgctgaacctttgaagtgttacctttccttgcaaaagattccgtatagaataggattaaaaattttcacaagttgtcagagaaaaataagaacagaaaattgaataaaatgtcagacctctggaaaatgaacagctttctcaaatttgaaaattaactataaaaaggaacagttttcctacggagacactgaggcgctctcagtgaaaaagaacgatgaaaaagaaccagaaaaggaaagaaaacggagttatgtatatgatttgtatctgatgcaaatttttcatacttgtgaaagaaaaatatcaagattataaaaagataaatggtgaaatgaagaatcatttatggaataaaatacaaatcaaagcaagtctggattatcgttttacaactactagtaaaaacagtaacagcaaccactcctggaaggttacctagaaatttgcatattcgtttatgtgaggtggcaaggctttggagttagaaatatggctctgcagctaattttacaatttgggacctaatttcgtcatcgtccttttgtccatttataaaatagaggaaattatacctacttcaggagtttgccaagattaactgtgtaaaactgacctttagcatgtatacatttattctttccctagtcacactgcactgggggacatttgtgaatctatgaaatttgtgaaaaatggatcctttaagccatgaccctgaaaccccactcctgggaacttacctgcaatggaagaaattcggaaagaagaaaagctgcattcacccacagggctcagaatgatctaaaattagatccagtccagagacaacctaaaggtattaagaaaatagcagggcagcagctaagaaaatcatagcactttaactgaaaggaacattgtgtaacccatcacgtggcataattttagagcctctctgtgatatataggaaaaaagtgacaggtcaaagtaagattactcagacatggatgcatatgtggaaaatctgaataaaaaatggacccacagttttctgtgtatgggaggagagttcagtgtcatgtttgctgcttttttttagtcagcgtcatctcttttaaaaatactatcatatttttttccttgagtagattcattagtggtttaataatttatatactgttattctattaaataatccgttcttagatttatcaattatagtttgtttttttttttaaggacttctgaatatatttgaaaactgaacagtttcaaccaagctgaagcatctgtcttcccagagacacagatccaacttgagctgaatcacagcagatgtaggtacctgcggaatctctctggtcttgtgatggttgaaagtgcccaactgtttcaagaagataagggactgaaagtctgggatcacaaatccttgctctggaggccactgggatctatatatgtaacccacacctattatatcactttcttgtaaaatcatcttgattttgcagggaaagggacatagctttctctagaatcattctgagttatgtaagaagtagccatttaaaaaacagtataataaaagcaataacctaacattcctgcaccgaatcaacactgaaggtaactgtcaacagacaaaaggttttatgaggtaacagtttttctaagctgtagttttaacttacgtattgcattctccctttttagatcttatttccttttccaaggcaaccagtttatcaactgtgaactgctacatatgaaacattcaaaacctgactgtgtctaaagctgtgatggctacagcaaaataatctttgagtgaatagcatgtttagcagttcttacagttgggagaattttttctcagtttgttcatccttctctcctaaccatgttctgcttattgctgtcctaatattgtgtgatcacgtcaagggaggtgttcccttttatgcaaaacattatgttaattgttttcttcctgagaacaagctcagaagattggctaggagtgcagttccctggaagccttattatagattactaaatcccatcactagataatcccaggctgttggcctgatgtagactctagctgtgttgcttttcttaaagctcttcacatcactctgaggatggaccagactggggactgtttgccctattcagtccagggttaggcctcagtgtcactggaaaaacctccacctcaaaatggtttgtaaatttttgtatagcttgcattagactctttataaggacagtgacctcaaaagatgaaaatatgacaaatgagttccacttagcttatgaaaaattggaaatgtccccagggcaaggatgggtagagggactgtttggtgccagtttccaatttaaataagtctcaagggtataacatattttgagtatcaaaaatttggctcctggcacatgaccactggacataagttcctaccagctctgattctcaatccccatgtataaaagggaataagatgaatgggacaatatatggattttgttgttgttgttccttctctctcattccatcgctctgccattgtgcttattcactaatgccacttgatcatatttattatagctttccaatccattctgatacttggcaggccaagtgtaccctagattttcttcctgttcagtaatttcttcagtcttcctaattttgagtatcactatatactataaaatcctctttgaattaggactgaaattgtattgacttaatgattaattggagttgaattggtatctttcaaaatcttcaatcttgattttcccaaccatacaccttgcctgtctttctttttttccagtcttccagcttttttggtctaagttctactatttattattagattaagtcttagtttgaattttgggttgccattaatgagtggaatttattttacacgctaaatctcctaagtaattacaactaattggagtatccactcttacctatatgtggagtccaaaaactgccaccctaataaataagctcattcatttatttgaagggatttggggatgattgccctgtatttcccagatagaaaatcatacctggctttctccatagctgtctaaaatagcagccatcacttacattcttgtcttgttaatctcattcatgagaatgctttcctctttatgttaagatagggtgtgggctgtgtattacttctagttctatagaaattttttcaacatctgaaacttaaaaagtcaggaaagcactgattcctgaatttaagtgagaactttgatttaattgaaaactttgcaacatcagagaatcttttttttctccttaacctactaataggttaattgatttcatgattctgagccattcttgtattcctaaaataatccttattggtcacagtgtattctttcactgaaatatcaaaatcaacttggtggtattttcttttggattttggcacccatattgctgaaggttgggtagctaagagtgtgagcccttcactcccctgtggctcttaaatgctacgtctcaggtgaaattgccccaaatggtttgtctctactgagtaaggagtttcagattccttataaccaattaccataaattggatgctcctcacttggaatgggctcatgggatggtaggcaaaggttaattttatccccaacctattaacttccaattttttcctcacaagggcctgggccccttccatcagtttttctcggtttttccaattaggtagcgtcagcaccatcctacctagttcccaaaagccaccaaacctggtcaactctgaactctctcatccactctatcagcttaaaattggttttttctacaaaatacatcccatttttcacagctccttgctgttttgtcacagtcccagattacaccaccatcttcctttaatcattttctaaatggtctcaccatttccattcttatccctcaaaattttctctttacacaacagtaagggtgaactttaaaaaaaagaaaaaaaaaaatctgtgatgtgattctcctaagccactttaatggcttcgcactgtttcaggctccagacgcttatctccctgtagtcctcaaacccacaaattaaatatcttaccactgtgtcctcagccacagaacagtaatattagaagacatgtaataaatatttgttcaagtaatcaatagttattgtacagtaacttttttctccatttcttttgatgtaaggtcaagttttccatttaaaaactggtacatactagctgtcccataaaaatttatttaatgattgattcaatgtcagtttttataattggatagaaaggtaatccctcccacacagatttttttttttttttaatgaggcaaggaatatgactacttggaaagctggctgcctgaaaaaatggcagattaatgtctcaaaacaaatatcagtgtctagacgccaggttcttttatagaacagagacagtaggaagatgaggaaataaaggcagaataaagagggagaagcggtgtggaagtcaagtaaaaaagacccgtgtcttgcaagacatctccagaaatggccagcctgtggaagggatatgttaatctcttctttcctgcaaccattcataggtgggaagtgtcagattatctccctgtgagctgaacaaaggcacttcagtccaacagttaggcagagggactgggttttctgaggcaggccattatatatgattataataacagcagcaacaaaaagcaagtcaaagaaacagttccaacatggagtcagaattggttcttctctgcaacagttccccactgtcaaggtccatttgacaatcttgtaggaaaaggggacagtgatctttctgtttgtcagttgaatctgggagtgtctgcctttggtaccagggttccaaatattgagatttgttttctttttggtctccttggaaagtgtctactttatacttgtcagcttagaaatattggacttcagacccaggagggtttctcaagttaagaaattttgcacttttctgtgtatgggaagatgcaagagtcaggactcattattatttccttgatatgtaattcagctgtctggggcctgtgatcctgtattctcaagtttcctcaaggttcaccatagggagtggtataatctggtgactgctagatgcaggtattctccttcctgagttttctttcctagttttctccttcctgagttgacttggggttcaccagctcacattagagggcttcagttgttgattctatttctcaggtcttcccttcttggtcaggaatttgactaatatttgggagacatttcatgatcaaactttgcttcacagtgctgtgaggttcatcccagatcaggcaaaacttcttgatgtaccactgcaggtgctaaattttggattagacccccatgaataattaaagattctctggattctgtctagttgtcatccaggagacattttccattgttgcttcttcccatacctagaatcacactattataattattttatgttataaatgtgatctattttctcaagatgtttagccgtagaagttttgttggaggcctgattatacattggttactgcaagagacaactatcgtataaattagtcaggatataagtaatgcagctagtaacactgataatgacatagtgcaacagggagatacaaaacgcaatttaaaaacgaagaatcaagtaaagcaatgattagtatgactagttgtagtccagttgcaataatctagtgaccacaggagccatagctgatttaacgagctagcttcagtttcattgtactggccgtggcatagcttaatctttgagacaagcatatgctactgggaggatcaaccagatagaaaaagatttaagttttacctttgcttacaaaggatataatttaccaaattactgtaagtcagaggttaaggaagttttccttacacctggaaaacacagatttaagccagttatttttagatagaaaccataaaaattataacaatttatcagttcactcagtcctatgtaactaatcctttttgttaacagctttatgaagccatcaagtttcccattagaattcttcaatgtattactagttcagcattacggtctaaaattcagaaacttggatttatccgaaagtcctttttataaaccttaaagaggaggcatttttacagaggcaacaaagtgaaaccataactgtataatgacaaaagacttaagaaggcactttaaatctgattatgatgcagttgacaaagtaacctggttactgctgtgacatacaatattttcaggtaataaaactaaaattatgactgataatattctaccaggacatagcacatttttaagaactccatataatctctagcatatcggtatcatttatcatataatttaagatttattattcattggataacacttgccatgtagtttaacataccaagtgaatctaattagtttaatatctccctttggtgtgtctcaggggccctttgaggcaccccaaagttagctaagatcaaaggaactttattgtaatttgatttgggaagtcttgtcaaaagagcattaagaaaaaatgttttaaaacacaacaggatcataggtcactgtgaaacaatatttacttagccaaaatgacaatgaatgatttcaaaggcaaatatagaacagattatttaaaaggtagaaatacttataatctgttatcaaaggagaagaaagccaaatttattttgtaccaagttactttcaagattcatttatttaattaaatttgtttcaaacttagtcctgatcatgtacaaaactactttttcagggtccatgtttcatgaactttccatcaattaatttattttagcacaattttaactttcaagttgttgaatatctggagatatcctaaaatataattatttctgaaagttcactcaaagctctaatcttcatttgcattttctttcccttaaaagtttcttcacatcaagtcccgttcgttactgacaaattgtatcaacaaacaaccatagataccaaatattaattaatattagatatttcccagctcacgtgaacctggagctcatttagcttaattgtatttagaattgtttggtttgtaagcacttacttttatttaaaccaattaaatagagctcttttacaaatcagctgcagcaaatgttatccaaagacaaagatacatacagagaggcctcaattcttatttcaagattgcagtcctgagtcaggcaatgtaaaaaaccatcagtttacatatgaggttgaattaaaattggatttctcatagatggaaaaaagtcaaactcacttggctagacagctaaatatttgcataaaaagcacttaggaattctaattatcttggccaacccaacttctaaattactttaccttctctaaaatttgcattttaaaaagacagcataatgagggttcctgagaagacatttgcatctcaacacaggcaagtttttcctaaaatgactttgtttcccctttgtttaatacttacaggcctcttaacataaccagggaggtctggggagtagtaaagttttgatgggatttggattatttttggaacgacacttctggtactgtaaagactacttttgtaaaacacagtttcgtgggggttttttttgaatgatactgaaggattgacatgcccatttacctatgttggaaagttttatctttcctcatttagcttagctcttgggaagacacagaggcaacaatttaggctcctgtaaatcagtccagactgaggggggaaagaggtgaagataagaaagacaggctgaggaatccgtctcatagtcctaccaggaagattgtagccagggggaatgggtcactgttttgcttgaaccaatgtgtgcttcacagtgcacagaagttgtcttgctggaattgttgtagccacaagttccgggaaacaaacttcactcagaaggacaatgcagatagtggagtacagttttattacaccggcgggcccaaggcagagtctcctcttagccaaggactctgaccagtttttgtgaaaaccttatatatcctaagtgtacgtgctcaaacccacctccccaaattccttgaaactagtctgaacacaagaaaagaaagatacaatcaaagttaacctgtgattcatatgccttaagcctaggtagttaacagtggacaattatcaataggcctgtggtcataccccaataagcataatagaatttatgattctattcagttatgcagataattagggtattcttttaggctactgagagtccagatatgagccctgggactcttccatctgggggggcggtgggtgggtctggttttcctgttagtgtatcattaccatagatactgggcatatagctcaaagtccgcagtccagcccaacatgggtcctgctttcaagatggagcctgttctgtttgtttcctccttcagagtcagatgctgtaatagtgtggtccattctgttacccacttatcctgtcacgagagacttcaagagacaggcaccttaatggcttttaagtgcctgacccacgccctcacaatattaattggcctggtactcagcctaagggagaaggaggaaaggagagccctgacctgcttgggaggcagctattggggcacagtaggctattaggccttcaaaacactccagagaataaccctagctagagttccatagctattagtctgtttgcagacggtttgagaagaaaggaatgagcgagtgtgaggagggtgaagatcttaaaggaagtacttctccttaatagctcaagcaattagtatcagatgtctgtgtgttaccaaagtatccagaataaaacaaaatctagccagctagagagtcacattaacatgacttcccagtttcattagacctgtgacctttgtccaaaatgctttataaatggaatttccttcacagggggtgctccccaagctgaagctgaagctctccactgtcaaagttacccagggctcctgtagggaaattagaatcagatgcctcaagtccaagggagtccccaagcctcttcatttattatcagagtgttcctctcctttgcaaaacacttctaattgcaagagtgtgtaattgtgagccattcaggcccattgcttttctgatcttagcatatctaatatatgtcccccaaagcttttcttcaggatagatgaaatatttcccatttttataagtttcatagcaccaaaacacacacaaaaataggtgaattccaacataaatgacacaaattccagtactaatacatagatgaaccagtttccagctcaggtaaataaatttatcctacaaaacaaatgaactaatcccaactgtgtgcttgttttgtgcccttcctctggaggcatgcatgctaagccgcttcaattgtgtctgactctttgcaactctttggactgtgtcccaccaggctcctctgtccatgggattctccaggcaagaatactgccatgcccttgatatactccagtattcttatactccagtatactctgttattcttgcctggagaatcccatggtcagaggagactgaaaggctacagttcatgaggtcatgaagagtaggacatgactgaaatgacatagcccaaaacaagcacacaactagggtaggcttgcccccaaaagaactagttccccaataaactggttccaactcaggtagagcccaacaacaattcccatctccaacagagtaccccaaccaaattggcttgtcctgtaaagaaaaagcccaaatttagaggggaatatgttctcagtgtgcacaccagagcaacttaaccctacaaaatcaagtttgtcaactcttaatagaaaagggcacacaaaaccacaaacaaatgagccagctatcgaatgaagaaaactaatgctatgaaattgggtctgttgacttgtagaagtttgttgattccttgcttcaactgccaggccctggggccactgatggcacttcagggaatcttgaaggagagatcctcagcacaaatggtcccagcagcttctagagccttgccctaacattccctaacagagctggctaaacacaagcagcaagtaaaatttgttaccaaatccagacttgctctactgagtgaacaacaggccagtgaatcagagatgagatgttgaaggaaagaatgtgactttattcagtaagctggctgaccgagaagatggcagactaacatcttaaaataaccatcttcttgggttctggatgccaggttctttacagaacagaaatgaggggaagtgaggaagtaaagtaaaaaggcagaatagagagggagaggcaataagtcttgggtcataagtcttgcaaaacatctctaggaatccccacacagttttttaattcataaaacttttaactttcacacttaggtctctaatccatttagagcctgcttttgcatgtgacattaaggtccgatttttattttccttcagtttcccagaagtctctgctaaataaactttcctttctcattgatttgttttgttagtttatcatttatccagttggtactaaagtcaatgtatgtggctttatctctgaacttgtcattctgcttcctttgatctatatgttcatttctaggttgttaccatatttattactatgactttatactaggatttaatgtttgataacagtaggtttttgaactgtggtattggagaagactcttgagagtcccttggactgcaaggagatccaaccggtccattctaaaggagatcagccctggaatttctttggaagaaatgatgctaaagctgaaattccagtactttggccacctcatgcaaagagttgactcattggaaaagactctgatgctgggagggattgggggcaggaggagaaggggacgacagaggatgagatggctggatggcatcaccgactcgatggacgtgagtctgagtgaactccgggagttattgatggacagggaggcctggcgtgctgcaattcatggggtcacaaagagtcggacacgactgagtgactgaactgaactgaacagtaggttctcacctcatttcctttttaaggttgagtttgttatttgtggacatttcttcatcctttattgaattcctcaaaaaaaaaatccagctagaattttgattgtcattattattcatattataagttcatttgtaggaaattgacatctttataatactgggtggttccaaccaagacctttccatgtttacagatcatcttctgtgttctttagtagtgttcagtttctttttccccagtcttactttcttttgagttaagtcaatcctagatactttacagtatgatagtgaaagttgtatctgactctttgtgaccccatggaccatacagcccatggaattctctaggccagaatactggagcgggtagcctttccttccaccagggcatcttcccaacccagggatcaaacccaggtctcccacattgcaggcaaattctttaccaggtgagccacaagggaagcccaagaatactggagtgggtagcttatcccttctccagtcggtcttcccgacccaggaatcaaactggggtttcctgcgttgtcggtgatttctttaccaactgagctatcaggaaagcccactttacagtatgggttgaaattattactatcttatctattaatttttttctaggcagttattgctggtatagagaaatgctgttgatttttataaaccaatccatagccttgctgaactctagttgagtctcctgttacctctgccaagtgtgtggaattttctatgtatatgatcacattaattccaaataatgacagctggagctcttttcttacagttattgtaccagtctttgtccttgcatatggcattggaaaagggcttcccaagtggctcagtgttaaagaatccacctatcaatgaggagatccaggtttgattcctgggtcaggaagatcccttggagaaggaaatggccacccactccagtgttcttgcctgggaaatctaatggacagaggagcctggagagctacagtccatggggtcacaagagtcggacacaacttagcaactaaaataacaataatggcactggaaggatctccagtcctgtgacaggggacatccttgttttgtttctgatcataaagggactgcattcaaacgttatctattaattatgtttaccatttctgttatataatctttattaagttaagcaggtttcctcctattcctagtctgctaagagtatttttcttagtgataggtatttagttcagttcagtcgctctgttgtgtcccactctttgcaacccccatggactgcagcatgccaggcttccctgtccatcaccaactcccagagtttactcaaactcatcatggtccattgagtcggtgatgccatccagtcatctcatcctctgtcatccccttctcctgccctcaatctttcccagcattagggtcttttaccatgatttctttgcatcaggtggccaaagcattggagtttcagcttcagcatcagtcctttcaatgaacacccaggactgatctcctttagaatggactggttggctctccctgcagtccaagggactctcaagagtcttttccaacataataacagttcaaaatctaggttggtcataactttccttccaaggagtaagcatcttttaatttcatggctgcagtcaccatctgcagtgattttggagcccccaaaataaagataggtattgactgttatcaaatacttaatatcttgatgtgctagaggattaggcagcaacccgactattctctgataacatcttatatgaagtgtcataacacagccaggcattcagaaacctaatgtgcattcctagcaattttactataatcacagatatgtttcttaaatttgctaatctttgtaacataatttgtacagtgagattttagattaaataatataagatcttccttattttattacaaacagaaaattctccaaggtctcattaagttttgagttcttcttgttcttgttcttgagttcttctagttcttctgattctctacttcaaaaaaattttttatgatttccataattaaatgttgtgctttgaatcccaaactggattttaaaatgatttggccttaatatagtttatgattatcccagctttattctaatattcagttttaaagtagtgatcagcaacatatgcccatggccttatgtcctgttgcctgtatctctagtccatgatcgttcctcagctccagactcaccaagcttaccagactcgctggtatctcttgtcccatgtgtagcttgataatggcattgtcaaagtgacactcttcatttgtcttcctatgggttggctccaccttccgtcttctgtccacagaattgtccaatccagaaatctggctgccgcctcaattcttctggtctttcatcttccatatcaaacaccaagccttcgagaaagttctcatgttcagttccctgaggtgtttgagcagaattgtgagcagcatggaagccaagagctgaaccccatgtgcaaatggagagtagatcagggaagcaggcctgcaaaagaccaggtgcaagaatgaggccatttggcatatcccaggaccctgtccctctggcccttacccaaaacagactccagaaattctagcagacatattctgagcagtttaaccggccttataactttcaaatgtatttttatttatacccaggcatttcagttatgatagtaaaaacataaaggggatgttaattcattgtttagtcatccttcctctgaatattatccaagttagtctttagttctgaaggtcatgaaaaataattttataatatttggtgacacttttatttgaagatgtcccagtgctgaggatgactaatgtcagcgttacagcatatgccatttttggttttgtggcaaatggtattttggaacatgtggtttgatgtggggtacagtagaaagtgcttaatgatcattctactgtgcgtctttaatctctgcccctggaaccacccagggtaagttagatattcattctgaaagatctgaatcttcaattcattcatctgtaatctgatgaatgtacattcacaacggttcataggttatcatgcagggtactttgttcccaaactgtgcttgcattatatgtaagatatgtgttttttgcaccaaaaattaagagaaaataagtcactcatgaaccattaaatgctgaactaagactcattcagtgagtgagtgactgcaaatattatgaacacagcctttcttaccccgttttgaatagcccgattgtctgtctatagaaagaaaaattactttataggtatgtttgcaaaatcttgcctgtttcctgtttccaaaaattattgtattgagaattcctttgataaaattcttgttggcatttatatgttcagaagatgttaattccttcatttaacagatatctattgtttaccttccctgtgccaggctctgccctggcccgctaagaagatagcagcaaacaaagagactcattccctgctcacattcccacatgaggaaagaggacatgaaccagctattcagaaaattatttcatgatctcagcacctaccttggggtcttcccaactggacattagaatcacttccatagggtccatgccagggttcagaaggttccaggaactaatatcccttataacaacccaataggcagagcttctagggtcctcacaagaacaagcccagttgcaagaatcactactttaaagaagttcaaagcgtggtaaacctaccagatatttatagtttcttccaatttatgatatagtgtaccaatcagaggttatttttatcataagcaatgttgctggcattctgtatttatcaagttactaggaaatgagccaggaattattttaaggtcaactttgtccttagaggagaaagagttgtgttactactttacctataattactttcatgagatgtatggaatgtgaagaacatttatgacctagaatgtttatagctgatgccactgctatacagtcattcattatgctgcagactttaagtgatttttacgtgggcatttgatgctgacaccctctttattttgcagagaagtcatcatggtgaaaagccacataggcagttggatcctggttctctttgtggccatgtggagtgacgtgggcctctgcaagaagcgaccaaaacctggcggaggatggaacactggggggagccgatacccgggacagggcagtcctggaggcaaccgctatccacctcagggagggggtggctggggtcagccccatggaggtggctggggccaacctcatggaggtggctggggtcagccccatggtggtggctggggacagccacatggtggtggaggctggggtcaaggtggtagccacagtcagtggaacaagcccagtaagccaaaaaccaacatgaagcatgtggcaggagctgctgcagctggagcagtggtagggggccttggtggctacatgctgggaagtgccatgagcaggcctcttatacattttggcaatgactatgaggaccgttactatcgtgaaaacatgtaccgttaccccaaccaagtgtactacagaccagtggatcagtatagtaaccagaacaactttgtgcatgactgtgtcaacatcacagtcaagcaacacacagtcaccaccaccaccaagggggagaacttcaccgaaactgacatcaagataatggagcgagtggtggagcaaatgtgcatcacccagtaccagagagaatcccaggcttattaccaaaggggggcaagtgtgatcctcttttcttcccctcctgtgatcctcctcatctctttcctcatttttctcatagtaggataggggcaaccttcctgttttcattatcttcttaatctttgccaggttgggggagggagtgtctacctgcagccctgtagtggtggtgtctcatttcttgcttctctcttgttacctgtataataatacccttggcgcttacagcactgggaaatgacaagcagacatgagatgctgtttattcaagtcccattagctcagtattctaatgtcccatcttagcagtgattttgtagcaattttctcatttgtttcaagaacacctgactacatttccctttgggaatagcatttctgccaagtctggaaggaggccacataatattcattcaaaaaaacaaaactggaaatccttagttcatagacccagggtccaccctgttgagagcatgtgtcctgtgtctgcagagaactataaaggatattctgcattttgcaggttacatttgcaggtaacacagccatctattgcatcaagaatggatattcatgcaacctttgacttatgggcagaggacattttcacaaggaatgaacataatacgaaaggcttctgagactaaaaaattccaacatatggaagaggtgcccttggtggcagccttccattttgtatgtttaagcaccttcaagtgatattcctttctttagtaacataaagtatagataattaaggtaccttaattaaactaccttctagacactgagagcaaatctgttgtttatctggaacccaggatgattttgacattgcttagggatgtgagagttggactgtaaagaaagctgagtgctgaagagttgatgcttttgaactatagtgttggagaaaactcttgagagtcccttggactgaaaggagatcagtcctgaatattcattggaaggactgatgctgaagctgaaactccaatactttggtcacctgatgggaagaactgaaggcaggagggatgctaggaaagactgaaggcaggaggagaaggggacgacagaggatgagatggctagatggcatcatggactcaatggacatgagcttaagtaaactccaggagttggcgatggacagggagacctggcgtcctgcagtccatggtgtcgcagagtcggacacgattgagtgactaaattgaggtgaacccagattttaacatagagaatgcagatacaaaaactccatattcatttgattgaatcttttcctgaaccagtgctagtgttggactggtaagagtataacagcatatataggttatgtgatgaagagaatagtgtacatgaaatatgtgcatttctttattgctgtcttataattgtcaaaaaagaaagttaggtccttggtttctgtaaaattgacttgaatcaaaagggaggcatttaaagaaataaattagagatgatagaaatctgatccattcagagtagaaaaagaaattccattactgttatttaagaaggtaaaattatttcctgaattgttcaatattgtcacctagcagatagacactattattctgtactgtttttactagcttgcaccttgtggtatcctatgtaaaaacgtatttgcatatgacaaactttttctgttagagcaattaacatctgaaccacctaatgcattacctgtttttgtaaggtactttttgtaaggtactaaggagatgtgggtttaatccctaggtcaggtaaatcccctagaggaagaaatggcaacccactccagtattcttgccaggaaaatccagtgggcagaggagcctggcagggtacagtctaagcatggggttgcaaagagtgagacaagacttgagctactgaacaataaggacaataaatgctgggtcggctaaaaggttcattaggttttttttctgtaagatggctctagtagtacttgtctttatcttcattcgaaacaattttgttagattgtatgtgacagctcttgtatcagcatgcatttgaaaaaaacatcaaaattggtaaatttttgtatagccatcttactattgaagatggaagaaaagaagcaaaattttcagcatatcatgctgtattatttcaagaaagataaccaaaatgcaaaaatgtatttgtgaagtgtatggagaaggggctgcaactgatcaagcttgtcaaagtagtttgtgaagtttcgtgctggagatttcttattggacgatgctccacagttggatataccagttgaagttgatagtgatcaaattgagatattgagaataatcgatgttataccacgcgggagatagctgacatactcaaaatatccaaatagaaccttgaaaaccatttgcaccatctcagttatgttaatcactttgatgtttgagttccacataagcaaaaaaacaacaacaacaaaaaaaaacacaaccttgaccatatttgcgcatgcagttctctactgaaatgattgaaaacactttgtttttaaaaacagattttgattaacagtgggtacgatacaataacgtagaatggaagaaattgtagggtgagcaaaatgaaccaccaccaccaaaggccagtcttcctctaaagaagatgtgtgtatggtgggattggaaagtaatcctctattatgaattcttctggaaaacactgctcctaattagaccaactgaaagcagcactcaacgaaaagcatccagaattagtcaatagaaaacataatcttccatcaggataacgcaagactacatatttctttgatgacccagcatggctggagtttctgattcatctgttgtattcagacgttgcatctttggattttttccatttatttcagtctacaaaattatcataatggaaaaaatttccattccctggaagattgtaaagtgcatctggaaaatttctttgctcaaaaagataaaaagttttgtgaacacagaattatgacgttgcctgaaaaatggcagaaggtagtggaacaaaagagtgactatgttgtttggtaaagttcttagtgaaaatgaaaaatgtgtcttttatttttatttaaacaccaaaggcacattttggccaacccaatactgaatacttaaaggaaactcttctgtgttgtccttagccttacagtgtgcactgaatagttttgtataagaatccagagtgatatttgaaatacgcatgtgcttatattttttatatttgtaactttgcatgtacttgttttgtgttaaaagtttataaatatttaatatctgactaaaattaaacaggagctaaaaggagtatcttccacggagtgtctggctgtgttcaccagtgtgcacactatgttggcagcttcatttggggggttaatatgagaaaagtgacacattcagtcctcacactgccaattgcagcaggagggctactcctgatcctgcttcagccttattcccagtcacatgccagctgttttctgctaccttttcacatttttccatgaatacctgtcaagtcactactatagcagaggaaaacagtccttgcattctggaagattttttctgtccaggattttgaaatagaggattttcttgattacgatgagacttaacaaagtatctaccttatgcctgtacccacccttgaaacactgtatggtcaatatggctactttcctttgttacaggctttcaaatggttcttcatatgtttcctccttcccaaatatgaggtaccagctcccagttttccttctcagaggttttcttctgcaactatagttcacttaacaggtggaagtaataaaaaaatgatagtggttgaaataatatttattaatactttagcaaatgccagtgtccttcagtatctgaacagaggatcaactttgcattaaaaatgtaaagattaaaaatcaacatcttgatatcccataattcacaaaactaattttaaaatgacataaaatcctcaaaagcattactcaaagttaaatctttaacataagaagtgctaggactattttcatgctgtccttttggccatatgcaagattatttaaaaataaactattcattatctgccagtcataatctcccaagaataccccactgaaaagatgtcagttatacaaagtaaggtatttacagagccgaagtgaatgatacacatctgtatctttctcaggctaccatgttttcttcctgttacttccagttcctttgagttgtgctaaagaaatttttttatattttatgtattttttaaaatggaggataattactttacaatattgtgatggtttctgccatacatcaacatgaatcggcatagggctttccaagtggcactagtggtaaagaactcacctgccaatgcaggagacctaagagatgtgggttcaatccctgagtcaggaagatgccctggaggagggcatggcaacccattccagtattcttgcctggagaatctccatggacagaggagcctggcgggccacagtccatagggctgcaaagagtcagacacaactgaagtgacttagcatgcatgtatacatatggccccttccctcttgaaccccctctaccacctccctccccaaccctctaggttgtcacagagcactagctttggtttccctgcatcatacgttgaactcccactggctctctgttttacatatggtatatgtttcagtgctattctctcatatcatctcgcactctccttcccttactgtgtccaaaatgtctgtgtttcctttgctgccctgcaagtaggaccatcattactatctttctagattccatatatatgtgttaatgtatgatatttgtctttctctttctaacttatttcactctataataggctctaggttcatccacctcactagaacagactcaaatatgttcctttttgtagctgagtaatattccattgtgtgtatgtaccacaacttcattatccattcatctatggttggacatctaggttgtttccatgtcctaggtattgtaaattgtgctgcaataaacattgaggtatatatgtctttttcagttctggtttcctcagggtataagcccagtagtgagattgctgggtcatatggtaactttgggctttccttgtggctctgctggtaaagaatccacctgcaatatgggagacctgggtttgatccctgggctgggaagaccccctggagaagggaacggctatccactccagtattctggcctggagaattccatggggtgtatagtccacggggttacaaagagttgcacacgactgagcaactttcactcactcatggtaactttatttctagttttttaaggaagctccataatgttctctatggtggctgtatcagtttgcattttgaccaacagtgtcagagggttcccttttctccacatcctctccagcatttattttttgtaaactttctgatgatggccattctgaccagtaataatgagtgatgttaagtatcttttcatgtgattattagtcatctgtcatctttggagaaatgtctgtttgggtcctctgcccattttaaaaatttggttgttttttgttattgagctgcatgaactgcttatatatttttgagattaattcctttcagttgtttcatttgcttattaatttctcccattcggagagttgtcttttcacctcgcttatggtttccttcattgtgacaaagcttttaagtttagttaggtcccacttatttatttttgtttttatttccattattctaggaagtgggtcaaagaggatcttgatgtggcttatgtcagatagtgttctgccctatgttttcctctaagagtcttatagtttctgatcttacatttaggtctttcatccatttgagtttatctttgtgtatggtgttaggaagtgttctaatttcattcttttactagtagctgaccagttttcccagtatgaattattgaagaggctatcttttctccattgtatatttttgcctcttttgtcaaagataaggtcctcataggtgcatggatttatctccaggctttctatattgttccattggtctatgcttccatttctgtgacagtatcatactgtcttgatgaccgtagctttgcagtatagtctgaagtcaggaaggttgattcctccatttccatttctttctcaagattgctttagctatttagggtcttttgtgtttccataaaattgtgagagtacttgttctagttctgtgaaaaataccattattagtttgatagggattgcattgaatctatagattgctttgggtaatatactcattttcactatattgattcttccaatccaagaacacggtatatttctgcatctgtttgtgttgtctttgatttatttctcaagtgtcttatagttttctgcatacaggtttttttgtctcttttggtaaatttattcctaggtattttcgttgttgcaatggtgaatgggattgtttccgtaatttctctttctgatttttcattgttagtgtataggaatgcaaggcatttctatgtattaattttatatcctatgactttactatattcattgattagctctagtaattttcgggtggcctctttagagttttatatgtagaggagcacataatctgcaaacagtgagagttttactacttcttttccaatctggattccttgtgttaaaggattttcactaaaaaattaaaataccaattttaaataactgagtctaactctcacagaaggtttttctggaggagtgtcaagtgtccaggtgtccaaacctttcctttccccctccctctctcttgggaggaaggtgtccccctacccttggaagggaaatttgggggcctgattgcttctcactctagtgggagccttaatatggccagaacctgagcttccctaggctcaggccctgaccttccattggtctaagaaactgacctacatagttcatttccacttgagaatggtcagttcctctctgctctttgaaactcctggaggatttagcatctcctgcattaattggaggagttaaacccatcctttgccacactcctgtgaggcctacccctgttctccaagaagccacacctctgctacacacacccagcctatgagcttcaactctgccttgctacaattttcctttcctggagagctggtgttctgttctttccctggagcagtgctcctcaaacttgaatgtgtgcctgactggagcccaagattctgcatttctcacaggttcccagatggtgccatgctggttctgtgaaacttcactggaacaactccctcaggatttcacactgaaacctctatcagcaccacctgaaggcttgttcaacacaagttgctgcatcccaccccagagtttctgattccagagtgcagggtaggaccagagaatttacatttctaacacactccccggcaatgatgctgttgatgtggagattgcaaatggagctccactgctctgcaggaagatgtacatgaaatagaaggtaaccatggcccctgaaaaatagagcagttaggagactaaaaacctgactggaacgctccctggggaggagagagctgagagctctagggatgaaaagcaaaggagatgtaaggaagtagttaatacctgcttcctgaaaaactggaaggactggtgagtcctgaggcccaccactagtgagagattcagctaaacttggaatagtacccaggccacaaatggagcatgtctcaaattcagatatgcacacaaatcacctgagaaccctgtcaaaatgcagttctgaggccatatgtttgatgtaagcttggagatttgtcatttctataagctcctgggtgatgtgtggtcccagtggtcccaggaccacaccaagaaacaaggacctagaagcctaagtcatctcttcacaccctggccaagactttgaagaaggattgaagtctcaggagctggggggagttggggagtagccaatagagagtctttaccttctcttgatttagccctaagctttgcctctcctgctttgagagcacattcctcttacctggcctgagttaaaagaatcaacctcctgccggcagcagtgaagtcagcttgtgtattatctcagaaacaagccgaattagttagctgcccatgggaaatatcaaatccagagacattctgtcagttttccaaggtcatacaaatagtgagtgaaaatgttagttgctcagtcatgtctgaccctttgcaaacttatggactatagctgccaggctcctctgtccatggaattctccaggcaagaatactggagtgggttgccataccctcctccaggggatc

SEQ ID NO: 4 - protein, 256 aa, Ovis aries
MVKSHIGSWILVLFVAMWSDVGLCKKRPKPGGGWNTGGSRYPGQGSPGGNRYPPQGGGGWGQPHGGGWGQPHGGGWGQPHGGGWGQPHGGGGWGQGGSHSQWNKPSKPKTNMKHVAGAAAAGAVVGGLGGYMLGSAMSRPLIHFGNDYEDRYYRENMYRYPNQVYYRPVDQYSNQNNFVHDCVNITVKQHTVTTTTKGENFTETDIKIMERVVEQMCITQYQRESQAYYQRGASVILFSSPPVILLISFLIFLIVG

SEQ ID NO: 5 - DNA, 830 bp, Odocoileus virginianus
acaccctctttattttgcagataagtcatcatggtgaaaagccacataggcagctggatcctagttctctttgtggccatgtggagtgacgtgggcctctgcaagaagcgaccaaaacctggaggaggatggaacactggggggagccgatacccgggacagggaagtcctggaggcaaccgctatccacctcagggagggggtggctggggtcagccccatggaggtggctggggccaacctcatggaggtggctggggtcagccccatggtggtggctgggggcagccacatggtggtggaggctggggtcaaagtggtacccacagtcagtggaacaagcccagtaaaccaaaaaccaacatgaagcatgtggcaggagctgctgccgctggagcagtggtagggggccttggtggctacatgctgggaagtgccatgagcagacctcttatacattttggcaatgactatgaggaccgttactatcgtgaaaacatgtaccgttaccccaaccaagtgtactacaggccagtggatcagtataataaccagaacacctttgtgcatgactgtgtcaacattacagtcaagcaacacacagtcaccaccaccaccaagggggagaacttcaccgaaactgacattaagatgatggagcgagttgtggagcaaatgtgcatcacccagtaccagagagaatcccaggcttattaccaaagaggggcaagtgtgatcctcttctcctcccctcctgtgatcctcctcatctctttcctcatttttctcatagtaggataggggcaaccttcctgttttcattatcttct

SEQ ID NO: 6 - protein, 256 aa, Odocoileus virginianus
MVKSHIGSWILVLFVAMWSDVGLCKKRPKPGGGWNTGGSRYPGQGSPGGNRYPPQGGGGWGQPHGGGWGQPHGGGWGQPHGGGWGQPHGGGGWGQGGTHSQWNKPSKPKTNMKHVAGAAAAGAVVGGLGGYMLGSAMSRPLIHFGNDYEDRYYRENMYRYPNQVYYRPVDQYNNQNTFVHDCVNITVKQHTVTTTTKGENFTETDIKMMERVVEQMCITQYQRESQAYYQRGASVILFSSPPVILLISFLIFLIVG

SEQ ID NO: 7 - DNA, 771 bp, Odocoileus hemionus hemionus
atggtgaaaagccacataggcagctggatcctagttctctttgtggccatgtggagtgacgtgggcctctgcaagaagcgaccaaaacctggaggaggatggaacactggggggagccgatacccgggacagggaagtcctggaggcaaccgctatccacctcagggagggggtggctggggtcagccccatggaggtggctggggccaacctcatggaggtggctggggtcagccccatggtggtggctgggggcagccacatggtggtggaggctggggtcaaggtggtacccacagtcagtggaacaagcccagtaaaccaaaaaccaacatgaagcatgtggcaggagctgctgcagctggagcagtggtagggggcctcggtggctacatgctgggaagtgccatgagcaggcctcttatacattttggcaatgactatgaggaccgttactatcgtgaaaacatgtaccgttaccccaaccaagtgtactacaggccagtggatcagtataataaccagaacacctttgtgcatgactgtgtcaacatcacagtcaagcaacacacagtcaccaccaccaccaagggggagaacttcaccgaaactgacatcaagatgatggagcgagttgtggagcaaatgtgcatcacccagtaccagagagaatcccaggcttattaccaaagaggggcaagtgtgatcctcttctcctcccctcctgtgatcctcctcatctctttcctcatttttctcatagtaggatag

SEQ ID NO: 8 - protein, 256 aa, Odocoileus hemionus hemionus; identical to SEQ ID NO: 6

SEQ ID NO: 9 - DNA, 830 bp, Cervus elaphus
acaccctctttattttgcagataagtcatcatggtgaaaagccacataggcagctggatcctagttctctttgtggccatgtggagtgacgttggcctctgcaagaagcgaccaaaacctggaggaggatggaacactggggggagccgatacccgggacagggaagtcctggaggcaaccgctatccacctcagggagggggtggctggggtcagccccatggaggtggctggggccaacctcatggaggtggctggggtcagccccatggtggtggctggggacagccacatggtggtggaggctggggtcaaggtggtacccacagtcagtggaacaagcccagtaaaccaaaaaccaacatgaagcatgtggcaggagctgctgcagctggagcagtggtagggggcctcggtggctacttgctgggaagtgccatgagcaggcctcttatacattttggcaatgactatgaggaccgttactatcgtgaaaacatgtaccgttaccccaaccaagtgtactacaggccagtggatcagtataataaccagaacacctttgtgcatgactgtgtcaacatcacagtcaagcaacacacagtcaccaccaccaccaagggggagaacttcaccgaaactgacatcaagatgatggagcgagttgtggagcaaatgtgcatcacccagtaccagagagaatccgaggcttattaccaaagaggggcaagtgtgatcctcttctcctcccctcctgtgatcctcctcatctctttcctcatttttctcatagtaggataggggcaaccttcctgttttcattatcttct

SEQ ID NO: 10 - protein, 256 aa, Cervus elaphus
MVKSHIGSWILVLFVAMWSDVGLCKKRPKPGGGWNTGGSRYPGQGSPGGNRYPPQGGGGWGQPHGGGWGQPHGGGWGQPHGGGWGQPHGGGGWGQGGTHSQWNKPSKPKTNMKHVAGAAAAGAVVGGLGGYMLGSAMSRPLIHFGNDYEDRYYRENMYRYPNQVYYRPVDQYNNQNTFVHDCVNITVKQHTVTTTTKGENFTETDIKMMERVVEQMCITQYQRESEAYYQRGASVILFSSPPVILLISFLIFLIVG

(19) United States
(12) Patent Application Publication
(10) Pub. No.: US 2002/0194635 A1
(43) Pub. Date: Dec. 19, 2002

(54) Transgenic animals resistant to transmissible spongiform encephalopathies

(76) Inventors: Patrick W. Dunne, La Grange, TX (US); Jorge Piedrahita, College Station, TX (US)

Correspondence Address: Robert E. Hanson, Fulbright & Jaworski L.L.P., Suite 2400, 600 Congress Avenue, Austin, TX, 78701, US

(21) Appl. No.: 10/109,551

(22) Filed: Mar. 28, 2002

Related U.S. Application Data
(60) Provisional application No. 60/280,549, filed on Mar. 30, 2001.

(51) Int. Cl. A01K 67/027

(52) U.S. Cl. 800/15

(57) ABSTRACT

The invention provides modified prion-encoding genes for the creation of transgenic bovine and cervid animals resistant to transmissible spongiform encephalopathies including bovine spongiform encephalopathy (BSE). The transgenic animals homozygous for the mutant genes continue to express a functional copy of the prion-encoding gene, thereby not interfering with the normal role of the polypeptide and effectively decreasing tendency for alteration of sleep-wake cycles.

This application claims the priority of U.S. Provisional Patent Application No. 60/280,549, filed Mar. 30, 2001, the entire disclosure of which is specifically incorporated herein by reference.

### BACKGROUND OF THE INVENTION

1. Field of the Invention

The present invention relates generally to the field of genetic transformation. More particularly, it concerns modification of a bovine prion protein gene useful in producing transgenic cattle exhibiting resistance to bovine spongiform encephalopathy.

2. Description of Related Art

Prions are highly infectious pathogens recognized as causing transmissible spongiform encephalopathies (TSEs) in humans and animals. Among the invariably fatal neurodegenerative diseases caused by these pathogens are bovine spongiform encephalopathy (BSE), scrapie in sheep and goats, chronic wasting disease in mule deer and elk, and Creutzfeldt-Jakob disease in humans. The pathogenic agent is an abnormal form of an endogenous protein (PrP^{C}), distinct from viruses and viroids in that prions are not associated with nucleic acids and appear to be composed entirely of an abnormal protein (PrP^{Sc}).

Prions are not associated with any nucleic acid and appear to be composed entirely of a modified protein (PrP). PrP exists in normal form in the cell but is believed to be converted to an abnormal form through a post-translational process resulting in a high beta-sheet content. Particular prions associated with a given species are encoded by the chromosomal PrP gene of the mammal in which it replicated. It is thought that prions embody strain specific properties in the tertiary structure of the modified prion protein. It is believed that the modified prion polypeptide acts as a template upon which normally occurring prion polypeptide is refolded into the modified form possibly facilitated by another protein (Prusiner S. B., 1998).

Bovine spongiform encephalopathy affects domestic cattle as a particular serious problem in the United Kingdom, France, Portugal and other European countries. The disease is invariably fatal for cattle, typically within weeks to months after becoming symptomatic. While BSE is associated with the transmissible agent, the precise mechanism of transmission is not well understood. A possible mode of transmission was believed to be the incorporation of sheep infected with scrapie in commercial cattle feed. In humans no direct link between CJD and BSE has been found but there is compelling evidence that a variant form of CJD may be caused by consumption of BSE contaminated beef (U.S. Pat. No. 5,737,061).

Symptoms of BSE in cattle commonly include changes in behavior such as unsteady gait or excessive nose licking. Recently, methods of diagnosis have been disclosed which relate the size of the pupil of the eye in conjunction with treatment of the animals prior to and subsequent to the administration of a neuro transmitter agonist or antagonist as differentiated from changes induced in the non-afflicted cattle (U.S. Pat. No. 5,737,061).

Prion protein encoding genes have been cloned, sequenced and expressed in transgenic animals. PrP^{C} for example is encoded by a single copy host gene and is normally found at the outer surface of neurons (Basler, et al., 1986). The biological function of PrP^{C} is not known, although it has been suggested that it is associated with acetyl coline receptor inducing activity (Harris, et al., 1991). The PrP gene is found in all mammals, including humans. The cause and mechanism of the transformation to the purportedly disease causing form is not known. However, certain mutations in the PrP gene such a proline to leucine change at position 102 have been linked to the disease in certain familial forms of spongiform encephalopathy (Hsaio and Prusiner, 1990). Mice carrying a PrP transgene with a proline to leucine change at position 102 develop a fatal scrapie-like disease.

More than twenty mutations of the PrP gene are now considered to cause the inherited human prion diseases and in some cases genetic linkages have been established for these mutations, for example, as described in Gabizon, et al., 1993.

Recombinant PrP mutated form has been produced. The isoform causing the disease may involve refolding of the residues within the region between residues 90 and 140 that form beta sheets. Anti-PrP Fabs have been selected from Phage Display Libraries and data from two monoclonal antibodies from hybridomas have led to the conclusion that the major conformational change that occurs during conversion of normal prion polypeptide into mutated polypeptide is located within a region bounded by residues 90 to 112 (Peretz, 1997). A currently unknown point mutations in PrP polypeptide without any known biological significance appear to occur either within or adjacent to regions of putative secondary structure in PrP polypeptide and may well destabilize the structure of PrP.

The entire open reading frame of all known mammalian and avian PrP genes resides within a single exon. The mouse, sheep, cattle and rat PrP genes contain three exons with the open reading frames in exon 3. Comparative sequencing of sheep and human cosmid clones containing PrP genes has revealed an additional putative small untranslated 5′ exon in the human PrP gene. Mapping of PrP genes to the short arm of human chromosome 20 and to the homologous region of the Mo chromosome 2 suggests the existence of PrP genes prior to the speciation of mammals. Mice expressing different levels of wild-type hamster PrP transgenes have been constructed inoculation of transgenic mice with prion disease forms of the hamster protein resulted in disease systems in the mice (Prusiner, 1998).

In view of the recent B SE epidemic in Great Britain, increased emphasis and study of prion strains and species barrier have been initiated. In cattle, the mean incubation time for BSE is approximately five years so that in a great majority of cattle harboring the disease which were slaughtered between ages 2 and 3 did not show manifestations of the disease. The origin of bovine prions that may have caused BSE cannot be determined from the amino acid sequence of the disease causing PrP polypeptide. The PrP.Sc in these animals has bovine sequence regardless of the source of the prions that may have caused the wild-type expressed PrP to alter its confirmation. Only one PrP polymorphism has been found in cattle. Most bovine PrP alleles encode 5 Octa repeats. Where 5 Octa repeats have been found, PrP alleles do not seem to be overexpressing BSE (Prusiner, 1998).

### SUMMARY OF THE INVENTION

A method has been developed to produce cattle that are expected to be resistant to bovine spongiform encephalopathy (BSE) without deleting a functional copy of the PrP gene. The method is applicable to all breeds of beef and dairy cattle. The bovine prion protein (PrP) gene confers susceptibility to scrapie-like agents from sheep or cattle that are responsible for the recent B SE epidemic in Britain (Anderson et al., 1996) The bovine gene was cloned and then modified by site-directed mutagenesis to produce a BSE-resistant form of the gene. The modified gene has been targeted to the location of the endogenous PrP gene in bovine fetal fibroblasts where it will replace the susceptible gene with the resistant form by homologous recombination.

The generation of transgenic cattle that are resistant to bovine spongiform encephalopathy (BSE) is accomplished by constructing a BSE-resistant prion protein (PrP) gene by site-directed mutagenesis. This is followed by in vitro conversion of the wild-type (susceptible) bovine PrP allele to a resistant allele by recombinant DNA technology and replacement of the wild-type allele by the resistant PrP allele in bovine fetal fibroblasts by homologous recombination. Live BSE-resistant cattle offspring from genetically manipulated fetal fibroblasts by nuclear transfer are then produced.

One aspect of the invention concerns a transgenic bovine comprising a transgene encoding a mutant PrP polypeptide comprising the polypeptide sequence of SEQ ID NO:2 in which an amino acid substitution has been made at position 171 of the sequence that renders the bovine resistant to bovine spongiform encephalopathy disease. Another embodiment of the invention concerns a transgenic bovine that comprises a mutated PrP polypeptide with an amino acid substitution in position 154 and/or 222. Such a substitution may be in place of or in addition to a substitution at position 171. In one embodiment of the invention, the amino acid substitution comprises substitution with an amino acid selected from the group consisting of histidine, lysine or arginine. The glutamine residue at position 171 of a transgenic bovine may be substituted with histidine, lysine or arginine. In one embodiment of the invention, the transgenic bovine is further defined as produced by a method comprising introducing a transgene encoding the mutant PrP polypeptide into the genome of a bovine embryo and allowing the embryo to develop into a bovine whose somatic and germ cells comprise the transgene.

The invention further provides a progeny of any generation of a transgenic bovine of the invention, wherein the progeny comprises the transgene. Still further provided is a fertilized embryo of a transgenic bovine of the invention, wherein the embryo comprises the transgene.

A transgenic bovine prepared in accordance with the invention may be further defined as lacking a functional wild type PrP gene. In one embodiment of the invention, a wild type PrP gene is replaced with a null allele by homologous recombination. The term “null allele” is understood by those of skill in the art to describe an allele which lacks function with respect to a wild type allele.

In another aspect of the invention, a method is provided of producing a transgenic bovine resistant to BSE comprising: a) introducing into a bovine embryo a transgene encoding a mutant PrP polypeptide comprising the polypeptide sequence of SEQ ID NO:2 in which an amino acid substitution has been made at position 171 of the sequence; and b) allowing the embryo to develop into a bovine the somatic and germ cells of which express the transgene, thereby rendering the transgenic bovine resistant to BSE. In the method, the mutant PrP polypeptide may further comprise an amino acid substitution at a position of the sequence selected from the group consisting of 154 and 222. In the method, the amino acid substitution may comprise substitution with an amino acid selected from the group consisting of histidine, lysine or arginine. In certain embodiments, the glutamine residue at position 171 has been substituted with histidine, lysine or arginine. In further embodiments, the transgenic bovine is further defined as lacking a functional wild type PrP gene and may be replaced with a null allele by homologous recombination.

In yet another aspect of the current invention, a transgenic cervid is provided comprising a transgene encoding a mutant PrP polypeptide comprising the polypeptide sequence of SEQ ID NO:2 in which an amino acid substitution has been made at position 171 of the sequence that renders the cervid resistant to cervid spongiform encephalopathy disease. Another embodiment of the invention concerns a transgenic cervid that comprises a mutated PrP polypeptide with an amino acid substitution in position 154 and/or 222. Such a substitution may be in place of or in addition to a substitution at position 171. In one embodiment of the invention, the amino acid substitution comprises substitution with an amino acid selected from the group consisting of histidine, lysine or arginine. The glutamine residue at position 171 of a transgenic cervid may be substituted with histidine, lysine or arginine. In one embodiment of the invention, the transgenic cervid is further defined as produced by a method comprising introducing a transgene encoding the mutant PrP polypeptide into the genome of a cervid embryo and allowing the embryo to develop into a cervid whose somatic and germ cells comprise the transgene.

The invention further provides a progeny of any generation of a transgenic cervid of the invention, wherein the progeny comprises the transgene. Still further provided is a fertilized embryo of a transgenic cervid of the invention, wherein the embryo comprises the transgene. A transgenic cervid prepared in accordance with the invention may be further defined as lacking a functional wild type PrP gene. In one embodiment of the invention, a wild type PrP gene is replaced with a null allele by homologous recombination.

In still yet another aspect of the invention, a method is provided of producing a transgenic cervid resistant to BSE comprising: a) introducing into a cervid embryo a transgene encoding a mutant PrP polypeptide comprising the polypeptide sequence of SEQ ID NO:2 in which an amino acid substitution has been made at position 171 of the sequence; and b) allowing the embryo to develop into a cervid the somatic and germ cells of which express the transgene, thereby rendering the transgenic cervid resistant to BSE. In the method, the mutant PrP polypeptide may further comprise an amino acid substitution at a position of the sequence selected from the group consisting of 154 and 222. In the method, the amino acid substitution may comprise substitution with an amino acid selected from the group consisting of histidine, lysine or arginine. In certain embodiments, the glutamine residue at position 171 has been substituted with histidine, lysine or arginine. In further embodiments, the transgenic cervid is further defined as lacking a functional wild type PrP gene and may be replaced with a null allele by homologous recombination.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1. Nucleic acid sequence (SEQ ID NO:1) and corresponding predicted amino acid sequence (SEQ ID NO:2) of wild-type bovine PrP and the boxed sequence representing the CAG to CGG mutation introduced at amino acid 179 (171), changing Gln to Arg.

FIG. 2. Verification of CAG to CGG mutation in the bovine PrP amino acid 171 codon changing a codon for Gln (CAG) to one coding for Arg (CGG). Asterisk indicates altered base.

FIG. 3. The PrP dominant negative transgene contains three elements: (1) an endogenous PrP promoter consisting of a portion of the 5′ UTR of bovine PrP gene, exon1, intron 1, exon 2 and the splice donor region of intron 2; (2) a 7.0 kb fragment containing a portion of intron 2 including the splice acceptor region of intron 2, and exon 3 modified at codon 171(179) to produce the dominant negative mutation Q171R; (3) a positive-negative neomycin-HSV-TK selection cassette.

FIG. 4. Targeting of the bovine PrP locus to generate a BSE-resistant null allele. The top line represents the normal PrP locus containing a promoter (Pr), three exons and a polyA addition site (pA). The second line represents the targeting vector that contains the promoterless selectable marker puromycin (puropA) cloned in-frame with PrP ORF. Homologous recombination between the targeting vector and the endogenous PrP locus results in substitution of the wild-type gene with the mutated gene, as illustrated on line 3.

FIG. 5. PCR diagnostics for targeting the PrP locus. a) endogenous gene. b) Targeted loci gains increase in size due to insertion of the puromycin gene. Primers 1r and 2r are outside the targeting construct. c) PCR results for targeted line (+1 and +2) and negative control (−1 and −2).

FIG. 6. Comparison of the PrP amino acid sequence among white tail deer (wtd) (SEQ ID NO:6), mule deer (md) (SEQ ID NO:6), elk (e) (SEQ ID NO:10), sheep (sh) (SEQ ID NO:4) and cattle (bov) (SEQ ID NO:2).

FIGS. 7, 7A. Cervid dominant negative substitutions at amino acids 154, 171 and 222 can be achieved in each case with a single base change to produce a resistant allele from a susceptible allele. The base change in each codon is underlined. The sequences represent the complete open reading frame for white tail deer (wtd) on line 1, elk (elk) on line 2, and mule deer (md) on line 3. The corresponding PrP nucleic acid sequences are given in SEQ ID NO:5, SEQ ID NO:9 and SEQ ID NO:7, respectively.

### DESCRIPTION OF ILLUSTRATIVE EMBODIMENTS

The invention overcomes the limitations of the prior art by allowing creation of PrP mutants that leave intact a functional copy of the PrP gene. Inactivation (knock-out) of the endogenous PrP (prion protein) gene in mice by homologous recombination produces animals that are healthy and capable of reproducing normally, while being resistant to spongiform encephalopathy (Bueler et al., 1993). Although the knock-out in mice leaves the affected mice physically normal, there is incontestable evidence of alteration in sleep-wake cycles and circadian rhythms (Tobler et al., 1996). Alteration in sleep regulation would likely have severe behavioral consequences for cattle. Caution is especially warranted since one of the inherited forms of human prion diseases, fatal familial insomnia, shows large changes in sleep and in the daily rhythms of several hormones.

Therefore, in the absence of knowledge of the normal function of this ubiquitously expressed protein, prudence would dictate maintaining a BSE-resistant but functional copy of the PrP gene in transgenic cattle. In order to create a resistant yet functional copy of the PrP gene in cattle, the disclosed procedures take advantage of the fact that a scrapie-resistance genotype already exists in sheep, a closely related member of the ruminant family. In sheep, where spongiform encephalopathy (scrapie) is an endemic disease, analysis of DNA derived from individual animals from infected flocks indicates that sheep resistant to the disease have a different PrP genotype from susceptible animals. In particular, the PrP genotypes Val 136, GLN 171 (PrP^{VQ}) and Ala 136, GLN 171 (PrP^{AQ}) have been shown to be associated with high susceptibility to scrapie and short survival times. In contrast, animals with Arg at position 171 (PrP^{VR} and PrP^{AR}) are resistant to infection and have incubation periods beyond their lifespan (Laplanche et al., 1993; Westway et al., 1994; Goldman et al., 1994; Clouscard et al., 1995; Belt et al., 1995). In the bovine, PrP is not polymorphic at the GLN 171 position, its genotype corresponding to PrP^{AQ} (Ryan and Womack, 1993, Hunter et al., 1994). Since bovine and sheep PrP are 98% identical at the amino acid level (Prusiner et al., 1993), it is highly likely that producing the same genotype in cattle that confers resistance in sheep (PrPAR), would be expected to show a similar level of resistance to the bovine spongiform encephalopathy. This change can be accomplished by a single amino acid substitution (GLNto Arg at position 171). Since this genotype in cattle is unknown in nature, this invention represents a novel method for producing cattle resistant to BSE.

I. Rationale and Significance of the Invention

The invention contributes to the art by providing mechanisms for the generation of animals resistant to TSEs. In this manner, the spread of such diseases can be eliminated. In a first aspect of the invention, a bovine or cervid animal is made resistance by expression of a transgene expressing a dominant-negative PrP protein. By introduction into a wild type background a resistant form of PrP, the protein can act in a dominant-negative manner and block production of amyloid particles.

A further aspect of the invention provides methods for the creation of TSE resistant animals by expression of a resistant form of PrP in a PrP minus background. In certain embodiments of the invention, this comprises generating a PrP deleted animal by homologous recombination to introduce into that animal the resistant form of PrP.

The invention also provides for the production of cervids expressing a resistant form of PrP in a wild-type and null background. Cervids contain a PrP that is 98% identical to sheep and bovine PrP at the amino acid level. Therefore, the inventors contemplate introducing the same mutations proposed for BSE resistance in cattle into cervids, including deer and elk species.

The invention is significant in that TSE diseases represent a critical and emerging issue to US and world agriculture. For example, the drastic effect BSE has had on the cattle industry in Europe, entry of TSE into a country's livestock population can be devastating. More importantly, it negatively influences the public perception of the safety of the animal food supply, and has long-term consequences for animal agriculture. It is imperative, therefore, that the tools of agricultural biotechnology and genomics are utilized to increase the level of safety of cattle populations both from a direct economic need, and a public perception need. In addition, with the emerging threat of bioterrorism, new technologies and approaches need to be developed to create safety mechanisms that can diminish or abolish such a threat. The approach described here can serve as a blueprint for future developments in related areas, and the information generated will benefit any future efforts to utilize the tools of biotechnology to improve the safety of our animal food supply.

Application to cervids is important because, unlike BSE, CWD is a rapidly propagating TSE in the United States with a natural mode of infectivity (horizontal transmission between animals). Moreover, a report documenting 3 unusually young patients with a TSE who regularly consumed venison raises the possibility of transmission of the disease to humans by consuming CWD-infected deer and elk (Belay et al., 2001). Although the economic impact of CWD may be smaller for the cervid industry, the specter of a human variant of CWD (perhaps not unlike the human BSE disease, new variant CJD) makes producing CWD-resistant animals even more urgent.

A. TSE Resistant Alleles

Bovine PrP encodes a protein of either 256 or 264 amino acids with 5 or 6 Gly/Pro-rich octapeptide repeats, respectively (Prusiner et al., 1993). High levels of expression of PrP are detected by Northern analysis in the brain, intermediate levels in heart and lung and low levels in the liver and spleen (Caughey et al., 1988). Inactivation of both endogenous PrP alleles in mice by homologous recombination results in animals that are completely resistant to spongiform encephalopathy (Beuler et al., 1993), although they may exhibit altered sleep-wake cycles and circadian rhythms (Tobler et al., 1996). Such observation is important as altered sleep regulation may have behavioral consequences for cattle.

Naturally occurring sheep PrP genotypes have been discovered that confer resistance to both experimental transmission of BSE and natural scrapie (LaPlanche et al., 1993; Goldman et al., 1994; Westaway et al., 1994. Clouscard et al., 1995; Belt et al., 1995; Foster et al., 2001) yet showed no abnormal behavioral or physiological phenotypes. In each case the resistant animals displayed either a Gln/Arg171 or Arg/Arg171 genotype. In addition, a human polymorphism that changes glutamic acid to lysine at amino acid 219 in human PrP also conferred resistance to classic CJD. Eighty-five CJD cases were examined and in all cases the genotype was Glu/Glu 219 although Glu/Lys 219 occurs in 12% of the general population (Shibuya et al., 1998). Overexpression of a resistant form of PrP in a susceptible background can act as a dominant negative mutation and interfere with the process of amyloid formation (Zulianello et al., 2000). This indicates that it may be possible to induce resistance by overexpression of a resistant form of PrP on a wild type background, as well as by replacement of the wild type version of the PrP for the resistant form. Thus it appears from both naturally occurring and experimentally induced changes at amino acids Q171R and Q222K manifest resistance to TSEs even in the heterozygous state.

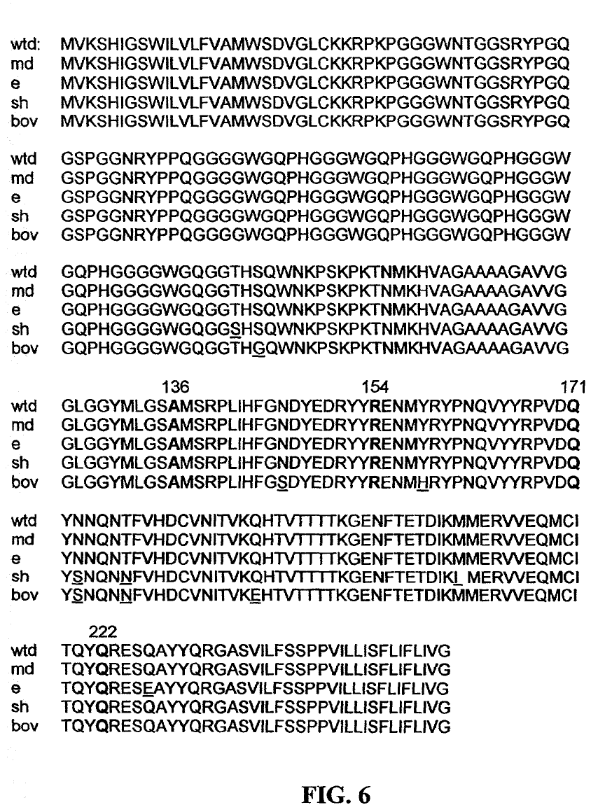

Sequence analyses of the cattle PrP gene consistently exhibit the susceptible allele at each of these sites. Chronic wasting disease is a TSE of free-ranging and captive deer and elk, confined mainly to the western US. As FIG. 6 illustrates, cervids, just like cattle, consistently exhibit the susceptible genotype Arg154Gln171Gln222 (Raymond et al., 2000).

B. Transgenic Cattle

While transgenic manipulation in mice have been very successful the same was not previously true for cattle. Fortunately, new advances in cloning by nuclear transfer have opened up a unique opportunity to undertake precise genetic modification in cattle. The ability of a number of different laboratory groups to successfully clone cattle is due to numerous research programs focused on nuclear transfer in cattle, and the base of knowledge developed over the last 20 years involving the application of assisted reproductive techniques in cattle. Successful and repeatable procedures for in vitro oocyte maturation, in vitro fertilization, and in vitro embryo culture are now well established for cattle and may find use in the creation of transgenic animals in accordance herewith.

Nuclear transfer has been used by the inventors to reproduce the genotypes of several animals, selected for cloning based on their inherent genetic value. Results obtained to date were similar to those reported by other laboratories. The first case involved a Brahman steer known to be at least 21 years old. Adult fibroblasts were obtained from a skin biopsy and expanded using standard methods for tissue culture prior to being frozen and stored in liquid nitrogen. When nuclear transfer was performed using the fibroblast cells derived from this animal, 28% of the fused couplets (53 of 190) developed into a blastocyst in culture. Twenty-six of these were transferred into 11 recipient cows resulting in 6 pregnancies. Three of these continued to develop through 90 days of gestation and one survived to term. This cloned Brahman bull is now 20 months old and appears normal and healthy for his age (13). The cloning of a Black Angus bull naturally (genetically) resistant to Brucellosis has also been achieved. Gene targeting technology has also been successfully developed in cultured fetal fibroblasts as described herein. Thus, the combination of the ability to undertake precise genetic modification in somatic cells and utilize those cells in a nuclear transfer procedure allows the creation of transgenic animals having mutated PrP genes.

II. Modified PrP Nucleic Acids and Polypeptides

One important aspect of the present invention concerns nucleic acids encoding modified PrP polypeptides and/or the creation and use of at least one recombinant host cell through the application of DNA technology, that expresses the mutant PrP polypeptide. Exemplary nucleic acids for modification include the coding sequence for the PrP gene of cattle, white tail deer, mule deer and elk are given in SEQ ID NO:1, SEQ ID NO:3, SEQ ID NO:5, SEQ ID NO:7 and SEQ ID NO:9, respectively. The corresponding polypeptides are given in SEQ ID NO:2, SEQ ID NO:4, SEQ ID NO:6, SEQ ID NO:8 and SEQ ID NO:10, respectively. An alignment of these polypeptide sequences is given in FIG. 6.

In certain aspects of the invention, polypeptides corresponding to these sequences are provided in which mutations have been made at selected residues including, for example, residues 154, 171, 222. As used herein, reference to these residues as “position 154”, “position 171” and/or “position 222” individually or collectively, refers to the homologous positions in these and other PrP sequences as indicated by the sequence alignment in FIG. 6. Thus it will be understood to those of skill in the art that various natural or synthetic alleles of the PrP gene that comprise additional or fewer amino acids than the sequences provided herein could be mutated at these corresponding positions and that the mutation will be made at the position that corresponds to the indicated homologous positions in FIG. 6. That is, the position numbers refer to the homologous positions as indicated in FIG. 6 but are not limited to the specified number of amino acid residues from the beginning of the translated polypeptide. These positions will be apparent to one of skill in the art based on the sequence of amino acids flanking each of the targeted positions for mutation.

The present invention concerns mutated DNA segments of PrP genes isolatable from bovines and cervids. As used herein, the term “DNA segment” refers to a DNA molecule that has been isolated free of total genomic DNA of a particular species. Therefore, a DNA segment encoding a mutated PrP polypeptide refers to a DNA segment that contains coding sequences yet is isolated away from, and/or purified free from, total genomic DNA. Included within the term “DNA segment”, are DNA segments and/or smaller fragments of such segments, and/or recombinant vectors, including, for example, plasmids, cosmids, phage, viruses, and/or the like.

As used herein, the term “nucleic acid” refers to a polymer of DNA, RNA or a derivative or mimic thereof, of two or more bases in length. It will be understood that the term “nucleic acid” encompass the terms “oligonucleotide” and “polynucleotide”. These definitions generally refer to at least one single-stranded molecule, but in specific embodiments will also encompass at least one double-stranded molecule. Within the scope of the invention, it is contemplated that the terms “oligonucleotide”, “polynucleotide” and “nucleic acid” will generally refer to at least one polymer comprising one or more of the naturally occurring monomers found in DNA (A, G, T, C) or RNA (A, G, U, C).

Similarly, a DNA segment comprising an isolated and/or purified PrP gene or polypeptide refers to a DNA segment including native or mutated PrP protein coding sequences and, in certain aspects, regulatory sequences, isolated substantially away from other naturally occurring genes and/or protein encoding sequences. In this respect, the term “gene” is used for simplicity to refer to a functional protein, polypeptide and/or peptide encoding unit. As will be understood by those in the art, this functional term includes both genomic sequences, cDNA sequences and/or smaller engineered gene segments that express, and/or may be adapted to express, proteins, polypeptides, domains, peptides, fusion proteins and/or mutants.

In particular embodiments, the invention concerns isolated DNA segments and/or recombinant vectors incorporating DNA sequences that encode a mutant PrP polypeptide that includes within its amino acid sequence a mutation at one or more residues selected from positions 154, 171 or 222. In particular embodiments, the mutation is at residue 171. Examples of such mutations include a change in the codon at position 171 of a PrP gene from glutamine to arginine. Further non-limiting examples of mutations include alteration of the arginine codon at position 154 to histidine and modification of the glutamine codon at position 222 lysine. Other modifications will also be known to those of skill in the art in light of the instant disclosure.

The term “a sequence essentially as set forth in” when used in combination with a reference to the SEQ ID NOS:2, 4, 6, 8, and/or 10, means that the sequence substantially corresponds to a portion of these sequences collectively or individually and/or has relatively few amino acids that are not identical to, and/or a biologically functional equivalent of, these amino acid sequences. In such instances the amino acid sequence may be about 98% identical to the polypeptide sequence of any of SEQ ID NOS:2, 4, 6, 8, or 10.

It will also be understood that amino acid and/or nucleic acid sequences may include additional residues, such as additional N- and/or C-terminal amino acids and/or 5′ and/or 3′ sequences, and/or yet still be essentially as set forth in one of the sequences disclosed herein.

Sequences that are essentially the same as those set forth in SEQ ID NOS:1, 3, 5, 7 and 9 may also be functionally defined as sequences that are capable of hybridizing to these sequences under relatively stringent conditions. Suitable relatively stringent hybridization conditions will be well known to those of skill in the art, as disclosed herein.

Hybridization is understood to mean the forming of a double stranded molecule and/or a molecule with partial double stranded nature. Stringent conditions are those that allow hybridization between two homologous nucleic acid sequences, but precludes hybridization of random sequences. For example, hybridization at low temperature and/or high ionic strength is termed low stringency. Hybridization at high temperature and/or low ionic strength is termed high stringency. Low stringency is generally performed at 0.15 M to 0.9 M NaCl at a temperature range of 20° C. to 50° C. High stringency is generally performed at 0.02 M to 0.15 M NaCl at a temperature range of 50° C. to 70° C. It is understood that the temperature and/or ionic strength of a desired stringency are determined in part by the length of the particular probe, the length and/or base content of the target sequences, and/or to the presence of formamide, tetramethylammonium chloride and/or other solvents in the hybridization mixture. It is also understood that these ranges are mentioned by way of example only, and/or that the desired stringency for a particular hybridization reaction is often determined empirically by comparison to positive and/or negative controls.

For applications requiring high selectivity, it is preferred to employ relatively stringent conditions to form the hybrids. For example, relatively low salt and/or high temperature conditions, such as provided by about 0.02 M to about 0.10 M NaCl at temperatures of about 50° C. to about 70° C. Such high stringency conditions tolerate little, if any, mismatch between the probe and/or the template and/or target strand, and/or would be particularly suitable for isolating specific genes and/or detecting specific mRNA transcripts. It is generally appreciated that conditions may be rendered more stringent by the addition of increasing amounts of formamide.

The nucleic acid segments of the present invention, regardless of the length of the coding sequence itself, may be combined with other DNA sequences, such as promoters, enhancers, polyadenylation signals, additional restriction enzyme sites, multiple cloning sites, other coding segments, and/or the like, such that their overall length may vary considerably. It is therefore contemplated that a nucleic acid fragment of almost any length may be employed, with the total length preferably being limited by the ease of preparation and/or use in the intended recombinant DNA protocol.

For example, nucleic acid fragments may be prepared that include a contiguous stretch of nucleotides identical to and/or complementary to the PrP coding sequences in SEQ ID NOS 1, 3, 5, 7 and/or 9. These sequences may then be operably linked to desired elements for heterologous expression, including promoter, or termination sequences.

In certain embodiments of the invention, modified PrP coding sequences may be prepared on transformation vectors. It will generally be preferable that the coding sequence be linked to a promoter or other regulatory element. In particular embodiments, the native PrP promoter may be preferred. Marker genes may be used, as is described herein.

The term “control sequences” refers to DNA sequences necessary for the expression of an operably linked coding sequence in a particular host organism. The control sequences that are suitable for prokaryotes, for example, include a promoter, optionally an operator sequence, a ribosome binding site, and possibly, other as yet poorly understood sequences. Eukaryotic cells are known to utilize promoters, polyadenylation signals, and enhancers.

Nucleic acid is “operably linked” when it is placed into a functional relationship with another nucleic acid sequence. For example, DNA for a sequence or secretory leader is operably linked to DNA for a polypeptide if it is expressed as a protein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous and, in the case of a secretory leader, contiguous and in reading phase. However enhancers do not have to be contiguous. Linking is accomplished by ligation at convenient restriction sites. If such sites do not exist, then synthetic oligonucleotide adapters or linkers are used in accord with conventional practice.

An “exogenous” element is defined herein to mean nucleic acid sequence that is foreign to the cell, or homologous to the cell but in a position within the host cell nucleic acid in which the element is ordinarily not found.

As used herein, the expressions “cell,” “cell line,” and “cell culture” are used interchangeably and all such designations include progeny. Thus, the words “transformants” and “transformed cells” include the primary subject cell and cultures derived therefrom without regard for the number of transfers. It is also understood that all progeny may not be precisely identical in DNA content, due to deliberate or inadvertent mutations. Mutant progeny that have the same function or biological activity as screened for in the originally transformed cell are included. Where distinct designations are intended, it will be clear from the context. “Plasmids” are designated by a lower case p preceded and/or followed by capital letters and/or numbers. The starting plasmids herein are commercially available, are publicly available on an unrestricted basis, or can be constructed from such available plasmids in accord with published procedures. In addition, other equivalent plasmids are known in the art and will be apparent to the ordinary artisan.

In certain embodiments of the invention, mutations are made in a PrP polypeptide by replacing one or more codons in the nucleic acid encoding the polypeptide. Such codons that may be used to make the changes are known to those of skill in the art. Mutagenesis may be carried out at random or, alternatively, particular identified sequences can be selectively mutated. In certain aspects of the invention, mutations are selectively made to the polypeptide residues 154, 171 and/or 222 of the PrP polypeptide. The means for mutagenizing a DNA segment comprising a specific sequence are well-known to those of skill in the art. Mutagenesis may be performed in accordance with any of the techniques known in the art, such as, and not limited to, synthesizing an oligonucleotide having one or more desired sequence.

Site-specific mutagenesis in particular will find use with the invention. The technique allows introduction of one or more nucleotide sequence changes into a DNA sequence. Site-specific mutagenesis allows the production of mutants through the use of specific oligonucleotide sequences which encode the DNA sequence of the desired mutation, as well as a sufficient number of adjacent nucleotides, to provide a primer sequence of sufficient size and sequence complexity to form a stable duplex on both sides of the deletion junction being traversed. Typically, a primer of about 17 to about 75 nucleotides or more in length is preferred, with about 10 to about 25 or more residues on both sides of the junction of the sequence being altered.

In general, the technique of site-specific mutagenesis is well known in the art, as exemplified by various publications. Various vectors have been used for site-specific mutagenesis, such as the M13 phage, as have double stranded plasmids. Alternatively, the use of PCR™ with commercially available thermostable enzymes such as Taq polymerase may be used to incorporate a mutagenic oligonucleotide primer into an amplified DNA fragment that can then be cloned into an appropriate cloning or expression vector. The PCR™-mediated mutagenesis procedures of Tomic et al. (1990) and Upender et al. (1995) provide two examples of such protocols.

The preparation of sequence variants of the selected coding DNA segments using site-directed mutagenesis is provided as a means of producing potentially useful species and is not meant to be limiting as there are other ways in which sequence variants of DNA sequences may be obtained. For example, recombinant vectors encoding the desired sequence may be treated with mutagenic agents, such as hydroxylamine, to obtain sequence variants.

Typically, vector mediated methodologies involve the introduction of the nucleic acid fragment into a DNA or RNA vector, the clonal amplification of the vector, and the recovery of the amplified nucleic acid fragment. Examples of such methodologies are provided by U.S. Pat. No. 4,237,224, incorporated herein by reference. A number of template dependent processes are available to amplify the target sequences of interest present in a sample, such methods being well known in the art and specifically disclosed herein.

In modifying a PrP gene it may be desired to consider the structure of the mutated polynucleotides and and/or proteins and other characteristics. For example, certain amino acids may be substituted for other amino acids in a protein structure without appreciable loss of interactive binding capacity with structures such as antigen-binding regions of antibodies, binding sites on substrate molecules, receptors, and such like. So-called “conservative” changes do not disrupt the biological activity of the protein, as the structural change is not one that impinges of the protein's ability to carry out its designed function. It is thus contemplated by the inventors that various changes may be made in the sequence of genes and proteins disclosed herein, while still fulfilling the goals of the present invention.

In terms of functional equivalents, it is well understood by the skilled artisan that, inherent in the definition of a “biologically functional equivalent” protein and/or polynucleotide, is the concept that there is a limit to the number of changes that may be made within a defined portion of the molecule while retaining a molecule with an acceptable level of equivalent biological activity. Biologically functional equivalents are thus defined herein as those proteins (and polynucleotides) in selected amino acids (or codons) may be substituted.

Amino acid substitutions are generally based on the relative similarity of the amino acid side-chain substituents, for example, their hydrophobicity, hydrophilicity, charge, size, and/or the like. An analysis of the size, shape and/or type of the amino acid side-chain substituents reveals that arginine, lysine and/or histidine are all positively charged residues; that alanine, glycine and/or serine are all a similar size; and/or that phenylalanine, tryptophan and/or tyrosine all have a generally similar shape. Therefore, based upon these considerations, arginine, lysine and/or histidine; alanine, glycine and/or serine; and/or phenylalanine, tryptophan and/or tyrosine; are defined herein as biologically functional equivalents.

To effect more quantitative changes, the hydropathic index of amino acids may be considered. Each amino acid has been assigned a hydropathic index on the basis of their hydrophobicity and/or charge characteristics, these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and/or arginine (−4.5).

The importance of the hydropathic amino acid index in conferring interactive biological function on a protein is generally understood in the art. It is known that certain amino acids may be substituted for other amino acids having a similar hydropathic index and/or score and/or still retain a similar biological activity. In making changes based upon the hydropathic index, the substitution of amino acids whose hydropathic indices are within ±2 is preferred, those which are within ±1 are particularly preferred, and/or those within ±10.5 are even more particularly preferred.

It also is understood in the art that the substitution of like amino acids can be made effectively on the basis of hydrophilicity, particularly where the biological functional equivalent protein and/or peptide thereby created is intended for use in immunological embodiments, as in certain embodiments of the present invention. U.S. Pat. No. 4,554,101, incorporated herein by reference, states that the greatest local average hydrophilicity of a protein, as governed by the hydrophilicity of its adjacent amino acids, correlates with its immunogenicity and/or antigenicity, i.e., with a biological property of the protein.

As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). In making changes based upon similar hydrophilicity values, the substitution of amino acids whose hydrophilicity values are within ±2 is preferred, those which are within ±1 are particularly preferred, and/or those within ±0.5 are even more particularly preferred.

III. Transgenic animals

Certain aspects of the invention concern the creation of genetically transformed cervid and bovine animals. Suitable methods of nucleic acid delivery for carrying out such transformation of a cell, tissue or an organism for use with the current invention are believed to include virtually any method by which a nucleic acid (e.g., DNA) can be introduced into a cell. Such methods include, but are not limited to, direct delivery of DNA such as by injection (U.S. Pat. Nos. 5,994,624, 5,981,274, 5,945,100, 5,780,448, 5,736,524, 5,702,932, 5,656,610, 5,589,466 and 5,580,859, each incorporated herein by reference), including microinjection (Harlan and Weintraub, 1985; U.S. Pat. No. 5,789,215, incorporated herein by reference); by electroporation (U.S. Pat. No. 5,384,253, incorporated herein by reference; Tur-Kaspa et al., 1986; Potter et al., 1984); by calcium phosphate precipitation (Graham and Van Der Eb, 1973; Chen and Okayama, 1987; Rippe et al., 1990); by using DEAE-dextran followed by polyethylene glycol (Gopal, 1985); by direct sonic loading (Fechheimer et al., 1987); by liposome mediated transfection (Nicolau and Sene, 1982; Fraley et al., 1979; Nicolau et al., 1987; Wong et al., 1980; Kaneda et al., 1989; Kato et al., 1991) and receptor-mediated transfection (Wu and Wu, 1987; Wu and Wu, 1988). Through the application of techniques such as these, organelle(s), cell(s), tissue(s) or organism(s) may be stably or transiently transformed. Specific, non-limiting examples of transformation methods that may be used with the invention are set forth herein below.

A. Site Specific Integration and Excision of Nucleic Acids

It is specifically contemplated by the inventors that one could employ techniques for the site-specific integration or excision of nucleic acids in connection with the instant invention. For example, site-specific recombination may find use in the elimination of selectable markers and/or for the replacement of a target loci in a genome. Site-specific integration or excision of nucleic acids can be achieved by means of homologous recombination (see, for example, U.S. Pat. No. 5,527,695, specifically incorporated herein by reference in its entirety). Homologous recombination is a reaction between any pair of DNA sequences having a similar sequence of nucleotides, where the two sequences interact (recombine) to form a new recombinant DNA species. The frequency of homologous recombination increases as the length of the shared nucleotide DNA sequences increases, and is higher with linearized nucleic acid molecules than with circularized plasmid molecules. Homologous recombination can occur between two DNA sequences that are less than identical, but the recombination frequency declines as the divergence between the two sequences increases.

Introduced DNA sequences can be targeted via homologous recombination by linking a DNA molecule of interest to sequences sharing homology with endogenous sequences of the host cell. Once the DNA enters the cell, the two homologous sequences can interact to insert the introduced DNA at the site where the homologous genomic DNA sequences were located. Therefore, the choice of homologous sequences contained on the introduced DNA will determine the site where the introduced DNA is integrated via homologous recombination. For example, if the DNA sequence of interest is linked to DNA sequences sharing homology to a single copy gene of a cell, the DNA sequence of interest will be inserted via homologous recombination at only that single specific site. However, if the DNA sequence of interest is linked to DNA sequences sharing homology to a multicopy gene of the host eukaryotic cell, then the DNA sequence of interest can be inserted via homologous recombination at each of the specific sites where a copy of the gene is located.

DNA can be inserted into the host genome by a homologous recombination reaction involving either a single reciprocal recombination (resulting in the insertion of the entire length of the introduced DNA) or through a double reciprocal recombination (resulting in the insertion of only the DNA located between the two recombination events). For example, if one wishes to insert a foreign gene into the genomic site where a selected gene is located, the introduced DNA should contain sequences homologous to the selected gene. A single homologous recombination event would then result in the entire introduced DNA sequence being inserted into the selected gene. Alternatively, a double recombination event can be achieved by flanking each end of the DNA sequence of interest (the sequence intended to be inserted into the genome) with DNA sequences homologous to the selected gene. A homologous recombination event involving each of the homologous flanking regions will result in the insertion of the foreign DNA. Thus only those DNA sequences located between the two regions sharing genomic homology become integrated into the genome.

One useful application of homologous recombination is the removal of selectable marker genes or other sequences that may be deemed undesirable for a particular application. One manner of removing sequences is to utilize a site-specific recombinase system. In general, a site specific recombinase system consists of three elements: two pairs of DNA sequence (the site-specific recombination sequences) and a specific enzyme (the site-specific recombinase). The site-specific recombinase will catalyze a recombination reaction only between two site -specific recombination sequences.

A number of different site specific recombinase systems are known and could be employed in accordance with the instant invention, including, but not limited to, the Cre/lox system of bacteriophage P1 (U.S. Pat. No. 5,658,772, specifically incorporated herein by reference in its entirety), the FLP/FRT system of yeast (Golic and Lindquist, 1989), the Gin recombinase of phage Mu (Maeser and Kahmann, 1991), the Pin recombinase of E. coli (Enomoto et al., 1983), and the R/RS system of the pSR1 plasmid (Araki et al., 1992). The bacteriophage P1 Cre/lox and the yeast FLP/FRT systems constitute two particularly useful systems for site specific integration or excision of transgenes. In these systems, a recombinase (Cre or FLP) will interact specifically with its respective site-specific recombination sequence (lox or FRT, respectively) to invert or excise the intervening sequences. The sequence for each of these two systems is relatively short (34 bp for lox and 47 bp for FRT) and therefore, convenient for use with transformation vectors.

The FLP/FRT recombinase system has been demonstrated to function efficiently in eukaryotic cells. In general, short incomplete FRT sites leads to higher accumulation of excision products than the complete full-length FRT sites. The systems can catalyze both intra- and intermolecular reactions, indicating its utility for DNA excision as well as integration reactions. The recombination reaction is reversible and this reversibility can compromise the efficiency of the reaction in each direction. Altering the structure of the site-specific recombination sequences is one approach to remedying this situation. The site-specific recombination sequence can be mutated in a manner that the product of the recombination reaction is no longer recognized as a substrate for the reverse reaction, thereby stabilizing the integration or excision event.

In the Cre-lox system, discovered in bacteriophage P1, recombination between loxP sites occurs in the presence of the Cre recombinase (see, e.g., U.S. Pat. No. 5,658,772, specifically incorporated herein by reference in its entirety). This system has been utilized to excise a gene located between two lox sites which had been introduced into a yeast genome (Sauer, 1987). Cre was expressed from an inducible yeast GAL1 promoter and this Cre gene was located on an autonomously replicating yeast vector.

Since the lox site is an asymmetrical nucleotide sequence, lox sites on the same DNA molecule can have the same or opposite orientation with respect to each other. Recombination between lox sites in the same orientation results in a deletion of the DNA Segment located between the two lox sites and a connection between the resulting ends of the original DNA molecule. The deleted DNA segment forms a circular molecule of DNA. The original DNA molecule and the resulting circular molecule each contain a single lox site. Recombination between lox sites in opposite orientations on the same DNA molecule result in an inversion of the nucleotide sequence of the DNA segment located between the two lox sites. In addition, reciprocal exchange of DNA segments proximate to lox sites located on two different DNA molecules can occur. All of these recombination events are catalyzed by the product of the Cre coding region.

B. Methods for Genetic Transformation

1. Electroporation

In certain embodiments of the present invention, a nucleic acid is introduced into an organelle, a cell, a tissue or an organism via electroporation. Electroporation involves the exposure of a suspension of cells and DNA to a high-voltage electric discharge. Recipient cells can be made more susceptible to transformation by mechanical wounding.

Transfection of eukaryotic cells using electroporation has been quite successful. For example, mouse pre-B lymphocytes have been transfected with human kappa-immunoglobulin genes (Potter et al., 1984), and rat hepatocytes have been transfected with the chloramphenicol acetyltransferase gene (Tur-Kaspa et al., 1986) in this manner.

2. Injection

In certain embodiments, a nucleic acid may be delivered to an organelle, a cell, a tissue or an organism via one or more injections (i.e., a needle injection), such as, for example, subcutaneously, intradermally, intramuscularly, intervenously, intraperitoneally, etc. Certain embodiments of the present invention thus include the introduction of a nucleic acid by direct microinjection. Direct microinjection has been used to introduce nucleic acid constructs into, for example, Xenopus oocytes (Harland and Weintraub, 1985).

3. Calcium Phosphate

In other embodiments of the present invention, a nucleic acid is introduced to the cells using calcium phosphate precipitation. Human KB cells have been transfected with adenovirus 5 DNA (Graham and Van Der Eb, 1973) using this technique. Also in this manner, mouse L(A9), mouse C127, CHO, CV-1, BHK, NIH3T3 and HeLa cells were transfected with a neomycin marker gene (Chen and Okayama, 1987), and rat hepatocytes were transfected with a variety of marker genes (Rippe et al., 1990).

4. DEAE-Dextran

In another embodiment, a nucleic acid is delivered into a cell using DEAE-dextran followed by polyethylene glycol. In this manner, reporter plasmids were introduced into mouse myeloma and erythroleukemia cells (Gopal, 1985).

5. Sonication Loading

Additional embodiments of the present invention include the introduction of a nucleic acid by direct sonic loading. For example, LTK⁻ fibroblasts have been transfected with the thymidine kinase gene by sonication loading (Fechheimer et al., 1987).

6. Liposome-Mediated Transfection

In a further embodiment of the invention, a nucleic acid may be entrapped in a lipid complex such as, for example, a liposome. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, 1991). Also contemplated is an nucleic acid complexed with Lipofectamine (Gibco BRL) or Superfect (Qiagen).

Liposome-mediated nucleic acid delivery and expression of foreign DNA in vitro has been very successful (Nicolau and Sene, 1982; Fraley et al., 1979; Nicolau et al., 1987). The feasibility of liposome-mediated delivery and expression of foreign DNA in cultured chick embryo, HeLa and hepatoma cells has also been demonstrated (Wong et al., 1980).

In certain embodiments of the invention, a liposome may be complexed with a hemagglutinating virus (HVJ). This has been shown to facilitate fusion with the cell membrane and promote cell entry of liposome-encapsulated DNA (Kaneda et al., 1989). In other embodiments, a liposome may be complexed or employed in conjunction with nuclear non-histone chromosomal proteins (HMG-1) (Kato et al., 1991). In yet further embodiments, a liposome may be complexed or employed in conjunction with both HVJ and HMG-1. In other embodiments, a delivery vehicle may comprise a ligand and a liposome.

7. Receptor Mediated Transfection

Still further, a nucleic acid may be delivered to a target cell via receptor-mediated delivery vehicles. These take advantage of the selective uptake of macromolecules by receptor-mediated endocytosis that will be occurring in a target cell. In view of the cell type-specific distribution of various receptors, this delivery method adds another degree of specificity to the present invention.

Certain receptor-mediated gene targeting vehicles comprise a cell receptor-specific ligand and a nucleic acid-binding agent. Others comprise a cell receptor-specific ligand to which the nucleic acid to be delivered has been operatively attached. Several ligands have been used for receptor-mediated gene transfer (Wu and Wu, 1987; Wagner et al., 1990; Perales et al., 1994; Myers, EPO 0273085), which establishes the operability of the technique. Specific delivery in the context of another mammalian cell type has been described (Wu and Wu, 1993; incorporated herein by reference). In certain aspects of the present invention, a ligand will be chosen to correspond to a receptor specifically expressed on the target cell population.

In other embodiments, a nucleic acid delivery vehicle component of a cell-specific nucleic acid targeting vehicle may comprise a specific binding ligand in combination with a liposome. The nucleic acid(s) to be delivered are housed within the liposome and the specific binding ligand is functionally incorporated into the liposome membrane. The liposome will thus specifically bind to the receptor(s) of a target cell and deliver the contents to a cell. Such systems have been shown to be functional using systems in which, for example, epidermal growth factor (EGF) is used in the receptor-mediated delivery of a nucleic acid to cells that exhibit upregulation of the EGF receptor.

In still further embodiments, the nucleic acid of a targeted delivery vehicle may be a liposome itself, which will preferably comprise one or more lipids or glycoproteins that direct cell-specific binding. For example, lactosyl-ceramide, a galactose-terminal asialganglioside, have been incorporated into liposomes and observed an increase in the uptake of the insulin gene by hepatocytes (Nicolau et al., 1987). It is contemplated that the tissue-specific transforming constructs of the present invention can be specifically delivered into a target cell in a similar manner.

C. Transgenic Animals

The current invention provides transgenic bovines and cervids. As used herein, the terms “cervid” or “cervids” includes deer and the like, including familiar moose, elk, and caribou. Members of this family occupy a wide range of habitats, from arctic tundras to tropical forests, and native species of cervids can be found over most of the world except Africa south of the Sahara, Australia, and Antarctica. They have also been introduced to a number of areas that originally had no cervids. Currently approximately 44 species of cervids are recognized.

As indicated herein above, the techniques of the present invention may also be used with potentially any bovine. As used herein, the terms “bovine” refers to a family of ruminants belonging to the genus Bos or any closely related genera of the family Bovidae. The family Bovidae includes true antelopes, oxen, sheep, and goats, for example. Preferred bovine animals are the cow and ox. Especially preferred bovine species are Bos taurus, Bos indicus, and Bos buffaloes. Other preferred bovine species are Bos primigenius and Bos longifrons.

Examples of particular cattle breeds that may find use with the invention include, but are not limited to: Aberdeen-Angus, Abigar, Abondance, Abyssian Highland Zebu, Abyssian Shorthorned Zebu, Aceh, Achham, Adarnawa, Aden, Afghan, Africander, Africangus, Agerolese, Alambadi, Ala-Tau, Albanian, Albanian Dwarf, Alberes, Albese, Aleutian wild, Alentejana, Aliad Dinka, Alistana-Sanabresa, Alur, American Angus, American Beef Friesian, American Breed, American Brown Swiss, American White Park, Amerifax, Amritmahal, Anatolian Black, Andalusian Black, Andalusian Blond, Andalusian Grey, Angeln, Angoni, Ankina, Ankole-Watusi, Aosta, Aosta Balck Pied, Aosta Chestnut, Aosta Red Pied, Apulian Podolian, Aracena, Arado, Argentine Crillo, Argentine Friesian, Armorican, Arouquesa, Arsi, Asturian, Atpadi Mahal, Aubrac, Aulie-Ata, Aure et Saint-Girons, Australian Braford, Australian Brangus, Australian Charbray, Australain Commercial Dairy Cow, Australain Friesian Sahiwal, Australain Grey, Australian Lowline, Australian Milking Zebu, Australian Shorthorn, Australian White, Austrian Simmental, Austrian Yellow, Avetonou, Avilena, Avilena-Black Iberian, Aweil Dinka, Ayrshire, Azaouak, Azebuado, Azerbaijan Zebu, Azores, Bachaur, Baggara, Baggerbont, Bahima, Baila, Bakosi, Bakwiri, Baladi, Baltic Black Pied, Bambara, Bambawa, Bambey, Bami, Banyo, Baoule, Bapedi, Bargur, Bari, Baria (Vietnam), Baria (Madagascar), Barka, Barotse, Barra do Cuanzo, Barrosa, Barroso, Barzona, Bashi, Basuto, Batanes Black, Batangas, Batawana, Bavenda, Bazadais, Beamais, Beefalo, Beefmaker (US), Beefmaker (Aussie), Beefmaster, Beef Shorthorn, Beef Synthetic, Beijing Black Pied, Beiroa, Beja, Belgian Black Pied, Belgian Blue, Belgian Red, Belgian Red Pied, Belgian White-and-Red, Belmont Red, Belted Galloway, Belted Welsh, Bengali, Bericiana, Berrendas, Bestuzhev, Betizuak, Bhagnari, Biamal, Black Baldy, Black Forest, Black Iberian, Blanco Orejinergo, Blauw and Blauwbont, Bleu du Nord, Blonde d'Aquitaine, Blonde du Sud-Ouest, Bolivian Criollo, Bonsmara, Boran, Borgou, Boreno Zebu, Braford, Bragado do Sorraia, Braganca, Brahman, Brahmin, Brahorn, Bralers, Bra-Maine, Brahmousin, Brandrood Ijsselvee, Brangus, Bra-Swiss, Bravon, Brazilian Dairy Hybrid, Brazilian Gir, Brazilian Polled, Brazilian Zebu, Breton Black Pied, British Dane, British Friesian, British Holstein, British Polled Hereford, British White, Brown Atlas, Brownsind, Bulgarian Brown, Bulgarian Red, Bulgarian Simmental, Burlina, Burmese, Burwash, Busa, Bushuev, Butana, Byelorussian Red, Byelorussian Synthetic, Cabannina, Cachena, Caiua, Calabrian, Cadeano, Caldelana, Calvana, Camargue, Cambodian, Canadien, Canary Island, Canchim, Cape Bon Blond, Caracu, Carazebu, Cardena, Carpathian Brown, Carrena, Casanareno, Cash, Casina, Castille-Leon, Caucasian, Caucasian Brown, Central American Dairy Criollo, Central Asian Zebu, Central Russian Black Pied, Chagga, Chan-Doc, Chaouia, Cahqueno, Charbray, Charford, Charolais, Charollandrais, Char-Swiss, Charwiss, Cheju, Chernigov, Chesi, Cheurfa, Chiangus, Chianina, Chiford, Chimaine, Chinampo, Chinese Black-and-White, Chino Santandereano, Chittagong, Cholistani, Cildir, Cinisara, Colombian Criollo, Coopelso 93, Cornigliese, Corriente, Corsican, Costeno con Cuernos, Cretan Lowland, Cretan Mountain; Croatian Red, Cuban Criollo, Cuban Zebu, Cukurova, Cuprem Hybrid, Curraleiro, Cutchi, Cyprus, Czech Pied, Dabieshan, Dacca-Faridpur, Dagestan Mountains, Dairy Gir, Dairy Shorthorn, Dairy Synthetic, Dairy Zebu of Uberaba, Dajjal, Damara, Damascus, Damietta, Danakil, Dangi, Danish Red Pied, Danish Blue-and-White, Danish Jersey, Danish Red, Danish Red Pied, Dashtiara, Dengchuan, Deoni, Devarakota, Devni, Devon, Dexter, Dexter-Kerry, Dhanni, Diali, Didinga, Dishti, Djakore, Dneiper, Doayo, Dobrogea, Dongola, Doran, Dorna, Dortyol, Drakensberger, Droughtmaster, Dun Galloway, Dutch Belted, Dutch Black Pied, East African Zebu, East Anatolian Red, East Anatolian Red and White, Eastern Nuer, East Finnish, East Friesian, East Macedonian, Ecuador Criollo, Egyptian, Enderby Island Shorthorn, Epirus, Estonian Black Pied, Estonian Native, Estonian Red, Ethiopian Boran, Faeroes, Fellata, Ferrandais, Fighting Bull, Finnish, Finnish Ayrshire, Flemish, Flemish Red, Florida Scrub, Fogera, Fort Cross, Franqueiro, Frati, French Brown, French Friesian, Friesland, Frijolillo, FRS, Gacko, Gado da Terra, Galician Blond, Galloway, Gambian N'Dama, Gaolao, Garfagnina, Garre, Gasara, Gascon, Gelbvieh, Georgian Mountain, German Angus, German Black Pied, German Black Pied Dairy, German Brown, German Red, German Red Peid, German Shorthorn, German Simmental, Ghana Sanga, Ghana Shorthorn, Gir, Giriama, Girolando, Glan, Glan-Donnersberg, Gloucester, Gobra, Gole, Golpayegani, Goomsur, Gorbatov Red, Goryn, Grati, Greater Caucasus, Greek Shorthorn, Greek Steppe, Grey Alpine, Greyman, Groningen Whitehead, Grossetana, Guadiana Spotted, Gaunling, Guelma, Guernsey, Gujamavu, Guzera, Guzerando, Hainan, Halhin, Hallikar, Hariana, Harton, Harz, Hatton, Hawaiian wild, Hays Converter, Hereford, Hereland, Herens, Highland, Hinterland, Hissar, Holgus, Holmonger, Holstein, Horro, Hrbinecky, Huangpi, Huertana, Humbi, Hungarian Grey, Hungarian Pied, Hungarfries, Ibage, Icelandic, Illawarra, Ilocos, Iloilo, Improved Rodopi, Indo-Brazilian Zebu, Ingessana, Inkuku, INRA 9, Iraqi, Irish Moiled, Iskar, Israeli Friesian, Istoben, Istrian, Italian Brown, Italian Friesian, Italian Red Pied, Jamaica Black, Jamica Brahman, Jamica Hope, Jamica Red, Japanese Black, Japanese Brown, Japanese Native, Japanese Poll, Japanese Shorthorn, Jarmelista, Jaulan, Javanese, Javanese Ongole, Javanese Zebu, Jellicut, Jem-Jem Zebu, Jenubi, Jerdi, Jersey, Jersian, Jersind, Jiddu, Jijjiga Zebu, Jinnan, Jochberg, Jotko, Kabota, Kabyle, Kachcha Siri, Kalakheri, Kalmyk, Kamasia, Kamba, Kamdhino, Kandahari, Kanem, Kangayam, Kaningan, Kankrej, Kaokoveld, Kappiliyan, Kapsiki, Karamajong, Karan Fries, Karan Swiss, Katerini, Kavirondo, Kazkh, Kazkh Whitehead, Kedah-Kelantan, Kenana, Kenkatha, Kenran, Kenya Boran, Kenya Zebu, Kerry, Keteku, Khamala, Kherigarh, Khevsurian, Khillari, Kholmogory, Khurasani, Kigezi, Kikuyu, Kilara, Kilis, Kinniya, Kisantu, Kochi, Kolubara, Konari, Korean Native, Kostroma, Kravarsky, Krishnagiri, Krishina Valley, Kuchinoshima, Kumamoto, Kumauni, Kurdi, Kurgan, Kuri, Kyoga, Ladakhi, Lagune, Lakenvelder, Las Bela, Latuka, Latvian Blue, Latvian Brown, La Velasquez, Lavinia, Lebanese, Lebedin, Lesser Caucasus, Liberian Dwarf, Libyan, Lim, Limiana, Limousin, Limpurger, Lincoln Red, Lithuanian Red, Llanero, Lobi, Local Indian Dairy, Lohani, Longhorn, Lourdais, Lowline, Lucanian, Lucerna, Lugware, Luing, Luxi, Macedonian Blue, Madagascar Zebu, Madaripur, Madura, Magal, Maine-Anjou, Makaweli, Malawi Zebu, Malnad Gidda, Malselv, Maltese cow, Malvi, Mampati, Manapari, Mandalong Special, Mangwato, Mantiqueira, Marchigiana, Maremmana, Marianas, Marinhoa, Maronesa, Maryuti, Masai, Mashona, Matabele, Maure, Mauritius Creole, Mazandarani, Mazury, Meknes Black Pied, Menufi, Merauke, Mere, Mertolenga, Messaoria, Metohija Red, Meuse-Rhine-Yssel, Mewati, Mezzalina, Mhaswald, Milking Devon, Milking Shorthorn, Mingrelian Red, Minhota, Miniature Hereford, Miniature Zebu, Minocran, Mirandesa, Mishima, Modenese, Modicana, Moi, Monchina, Mongalla, Mongolian, Montafon, Montbeliard, Morang, Morenas del Noroeste, Morucha, Mottled Hill, Mozambique Angoni, Mpwapwa, Munshigunj, Murcian, Murgese, Murle, Murnau-Werdenfels, Murray Grey, Muris, Muturu, Nagori, Nakali, Nama, Nandi, Nantais, Nanyang, Ndagu, N'Dama, N'Dama Sanga, Nejdi, Nelore, Nepalese Hill, N'Gabou, Nganda, N'Gaoundere, Nguni, Nilotic, Nimari, Nkedi, Nkone, Normande, Normanzu, North Bangladesh, North Finnish, North Malawi Zebu, North Somali, Norwegian Red, Nuba Mountain, Nuer, Nuras, Nyoro, Okayama, Ongole, Oran, Orapa, Oulmes Blond, Ovambo, Pabna, Pajuna, Palmera, Pakota Red, Pantaneiro, Pantelleria, Paphos, Parthenias, Pechora, Pee Wee, Peloponnesus, Perijanero, Pester, Philippine Native, Piedmont, Pie Rouge de l'Est, Pie Rouge des Plaines, Pinzgauer, Pinzhou, Pisana, Pitangueiras, Polish Black-and-White Lowland, Polish Red-and-White Lowland, Polish Simmental, Polled Charolais, Polled Gir, Polled Guzera, Polled Hereford, Polled Jersey, Polled Lincoln Red, Polled Nelore, Polled Shorthorn (US), Polled Simmental, Polled Sussex, Polled Welsh Black, Polled Zebu, Poll Friesian, Poll Hereford, Poll Shorthorn (Aussie), Pontremolese, Ponwar, Porto Amboim, Posavina, Preti, Prewakwa, Puerto Rican, Pul-Mbor, Punganur, Purnea, Pyrenean, Qinchuan, Quasah, Ramgarhi, Ramo Grande, Rana, Randall Lineback, Ranger, Rath, Raya-Azebo, Red and White Friesian, Red and White Holstein, Red Angus, Red Belted Galloway, Red Bororo, Red Brangus, Red Chianina, Red Desert, Red Galloway, Red Kandhari, Red Poll, Red Sindhi, Red Steppe, Reggiana, Regus, Rendena, Renitelo, Retinta, Rhaetian Grey, Rio Limon Dairy Criollo, Riopardense, Rodopi, Rojhan, Romagnola, Roman, Romana Red, Romanian Brown, Romanian Red, Romanian Simmental, Romanian Steppe, Romosinuano, Russian Black Pied, Russian Brown, Russian Simmental, Rustaqi, RX3, Sabre, Sahford, Sahiwal, Saidi, Salers, Salorn, Sanhe, San Martinero, Santa Gertrudis, Sarabi, Sardinian, Sardinian Brown, Sardo-Modicana, Savinja Grey, Sayaguesa, Schwyz-Zeboid, Seferihisar, Senepol, Sengologa, Serbo-Cro Pied, Serbo-Cro Pinzau, Serere, Seshaga, Shahabadi, Shakhansurri, Shandong, Sharabi, Sheko, Shendi, Shetland, Shimane, Shkodra, Shuwa, Siberian Black Pied, Siberian White, Siboney, Simbrah, Simford (Australia), Simford (Israel), Simmalo, Simmental, Sinhala, Siri, Sistani, Slovakian Pied, Slovakian Pinzgau, Slovenian Brown, Slovenian Podolian, Small East African Zebu, Socotra, Sokoto Gudali, Somali, Somba, Sonkheri, Son Valley, South African Brown Swiss, South Anatolian Red, South China Zebu, South Devon, Southern Tswana, Southern Ukrainian, South Malawi Zebu, Spanish Brown, Spreca, Sudanese Fulani, Suia, Suisbu, Suk, Suksun, Sunkuma, Sunandini, Sussex, Swedish Ayrshire, Swedish Friesian, Swedish Jersey, Swedish Mountain, Swedish Polled, Swedish Red-and-White, Swiss Black Pied, Swiss Brown, Sychevka, Sykia, Tabapua, Tagil, Taino, Taiwan Zebu, Tajma, Tamankaduwa, Tambov Red, Tanzanian Zebu, Tarai, Tarentaise, Tarina, Taylor, Telemark, Texas Longhorn, Thai, Thailand Fighting cow, Thanh-Hoa, Thari, Thatparkar, Thessaly, Thibar, Thillari, Tibetan, Tinima, Tinos, Tonga, Toposa, Toro, Toronke, Tottori, Toubou, Toupouri, Transylvanian Pinzgua, Tropical, Tropical Dairy Cattle, Tropicana, TSSHZ-1, Tawana, Tudanca, Tuli, Tuni, Turino, Tukana, Turkish Brown, Turkish Grey Steppe, Turkmen, Tux-Zillertal, Tuy-Hoa, Tyrol Grey, Uganda Zebu, Ujumqin, Ukrainian Grey, Ukrainian Whiteheaded, Umblachery, Ural Black Pied, Valdres, Vale and Vaalbonte, Vaynol, Vendee Marsh, Venezuela Criollo, Venezuelan Zebu, Verinesa, Vianesa, Victoria, Vietnamese, Villard-de-Lans, Vogelsberg, Volnsk, Voderwald, Vosges, Wakwa, Watusi (USA), Welsh Black, Wenshan, West African Dwarf Shorthorn, West African Shorthorn, West Finnish, West Macedonian, Whitebred Shorthorn, White Caceres, White Fulani, White Galloway, White Nile, White Park, White Sange, White Welsh, Witrik, Wodabe, Wokalup, xinjiang, Xuwen, Yacumeno, Yakut, Yanbian, Yaroslavl, Yellow Franconian, Yemeni Zebu, Yunnan Zebu, Yurino, Zambia Angoni, Zanzibar Zebu, Zaobei, Zavot, and Znamensk.

### IV. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Bovine PrP gene

The bovine PrP gene was isolated from a bacterial artificial chromosome (BAC) large-insert library by PCR™ screening of pooled clones (Cai et al., 1995). Three overlapping BAC clones were verified to contain the PrP gene by dideoxy sequencing. Since the efficiency of the process of homologous recombination depends on the targeting construct containing DNA that is identical (“isogenic”) to the DNA at the targeted locus, the PrP gene was amplified by long-range PCR™ (LR-PCR™). Long-range PCR™ has been used previously for obtaining isogenic DNA for the generation of targeting constructs (Randolph et al., 1996). The technology is based on a combination of DNA polymerases and can produce amplicons as large as 35 kb (Barnes, 1994).

More important than the length of homology is the remarkable fidelity of the enzymes with error rates as low as 1.3×10⁻⁶ per bp per replication (Stratagene). Thus a conservative error rate would be 1 bp mismatch every 30-40 kb. Such a low level of mismatch should not affect the targeting frequency as has been demonstrated by Randolph et al., (1996) who indicated that the targeting frequency did not differ between constructs made the conventional way (cloning of the genomic sequence), versus targeting vectors prepared by long-range PCR™.

In addition, the sequencing done to confirm identity of several long-range PCR™ products has not identified a single mismatch in over 5 kb of sequences. The proposed method, therefore, is not only technically feasible but is not expected to result in lower targeting rates. The ends of a portion of the cloned PrP gene were sequenced and the sequence data used to synthesize PCR™ primers. As a source of isogenic bovine genomic DNA, fetal fibroblasts were first isolated from a genetically superior Aberdeen Angus 40-day old fetus and DNA extracted by standard procedures. Using optimized LR-PCR™ conditions, a 7 kb isogenic PrP fragment was amplified and cloned into vector pCR2. 1, the insert digested with the restriction enzymes BamHI and HindIII and the resulting 3.0 kb fragment subcloned into vector pUC 19.

A. Use of Isogenic Targeting DNA Generated by Long-Range PCR™.

This feature allows rapid generation of isogenic DNA for construction of a targeting vector without having to laboriously construct and screen a genomic library. Since the PCR™ product is identical in sequence to the targeted endogenous gene, it facilitates homologous recombination.

B. Use of superior genetics in the fetus used as the source of fetal fibroblasts.

This feature will make the PrP-resistant transgenic calves that the inventors produce much more valuable to the cattle industry as breeding stock.

### Example 2

### Modification of PrP Gene

Since the desired alteration in the bovine PrP gene is a one base change at amino acid 176 (CAG coding for glutamine to CGG coding for arginine), this modification was introduced by in vitro mutagenesis following the methodology of Deng and Nickoloff (1991).

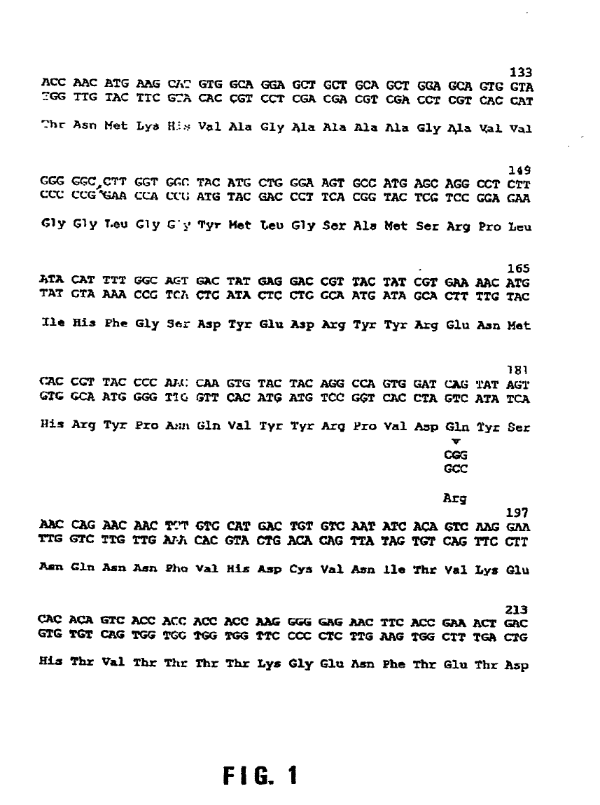

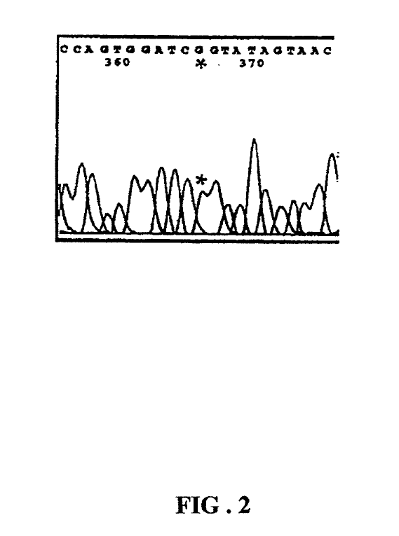

In order to convert the wild type triplet sequence (CAG) coding for glutamine (Gln) at amino acid 179 to arginine (Arg) coded by the triplet CGG, a mutagenesis primer was synthesized that would convert the middle base of CAG from A to G thereby producing the required CGG triplet coding for Arg rather than Gln (See FIG. 1).

The mutagenesis protocol of Deng and Nicholoff was used to generate the amino acid change indicated above. Briefly, the vector containing wild-type PrP was denatured and then hybridized with the mutagenesis primer that encodes the desired modification (C to G) and a selection primer that alters a unique restriction site. A second strand is synthesized using DNA polymerase and gaps sealed with DNA ligase. Following transformation into an appropriate host, clones containing the mutagenic plasmid are selected by digestion of isolated DNA with the restriction enzyme that digests the altered site.

Plasmids were isolated from individual colonies and digested with EcoRV to distinguish mutated plasmids from parental ones. To confirm that a C to G change had been introduced into PrP (and no other unintended changes introduced), several sequencing primers were synthesized allowing sequencing of the entire coding region on both strands. Only the C to G base change was detected. Since the final transgene was to contain 7 kb of PrP DNA, the mutated region was ligated to a plasmid vector (pBluescriptKS) containing 7.0 kb of PrP gene, replacing the 3 kb fragment containing the wild-type allele with the mutated allele. Sequence analysis confirmed that the mutated allele had been successfully subcloned into the 7 kb PrP DNA fragment. A portion of the endogenous PrP promoter was ligated upstream of the mutated PrP gene and a double selectable marker cassette consisting of the neomycin (neo) and thymidine kinase (TK) gene was ligated downstream of the altered PrP gene. Using this double cassette it is possible to select for the presence (neo) as well as the absence (TK) of the selection cassette. This cassette, in addition, is flanked by small regions of DNA known as loxP sites which act as recognition sites for a DNA recombinase enzyme known as Cre, permitting excision of the cassette prior to cloning.

A. Gene Modification in Null Background Rather Than Simple Gene Knock-Out

One difference in the inventors' approach versus the patent of Weissmann et al. (U.S. Pat. No. 5,698,763) is that the modification the inventors have created will leave intact a functional copy of the PrP gene in a null background.

B. Use of loxP-Mediated Excision of Selectable Markers

Removal of markers that confer antibiotic resistance (neo) or sensitivity to toxins (TK) may be a regulatory requirement for transgenic animals. Such markers may be readily eliminated in the appropriate constructs.

### Example 3

### Electroporation of Dominant Negative Transgene and Nuclear Transfer

The transgene is electroporated into fetal fibroblast cells collected from 40 day-old fetuses derived from genetically superior parents. The transgene may be introduced into a wild-type background or may be electroporated into modified fibroblasts where either one or both PrP alleles have been knocked out (See Example 5 below). Cells resistant to neomycin are expanded, a fraction frozen for future nuclear transfer studies, and the remainder expanded and used for isolation of DNA.

Once transgenic cells are identified, the cells are expanded again, electroporated with a cre-expressing plasmid, and cultured in the presence of ganciclovir. Cells that have lost the TK marker due to the cre-mediated excision will survive in ganciclovir while the other cells will die. The result of the event is a PrP dominant negative transgene expressing a BSE-resistant form of PrP locus identical to the original one except for the amino acid substitution introduced in vitro.

Previous results have indicated that early embryonic and fetal cells have a greater chance of participating in normal embryonic development after nuclear transfer (NT) than do adult somatic cells. Sufficient cells can be obtained from a single fetus to observe even a rare gene targeting event, making it is possible to use superior genetics. This assures that any animals produced have not only the genetic mutation conferring resistance to PrP, but also the genetic potential to perform at the top of their breed.

Cloning of cattle by nuclear transfer using fetal fibroblasts as nuclear donors has been demonstrated by two laboratories (Cibelli et al., 1998; Wells et al., 1998). Since in each case the fetal fibroblast donor cells were genetically modified prior to nuclear transfer, it is reasonable to believe that their manipulation will not substantially alter the efficiency of the NT technology.

### Example 4

### Inactivation of PrP Gene in Mice

Creation of transmissible spongiform encephalopathy (TSE)-resistant livestock can be accomplished by knocking out both copies of the PrP gene by homologous recombination.

As an in vivo model, the PrP gene of mouse ES cells was disrupted using conventional targeting protocol where a portion of PrP was replaced with a ncoTK cassette. Chimeric offspring were mated to produce homozygous PrP mice (Bueler et al., 1992). When challenged with mouse scrapie prions, the mice remained free of scrapie symptoms (Bueler et al., 1993). This method has the advantage that it has been demonstrated that in mice this knock-out event makes animals resistant to challenge from exogenous sources of virulent TSE isoforms that are highly infectious in otherwise identical mice having the normal pair of PrP alleles.

The major drawback to this approach is the possible physiological and behavioral consequences of eliminating the functioning of a ubiquitously expressed gene. Evidence has now emerged that these mice exhibit profound alterations in day/night rhythms and sleep patterns which might be anticipated to cause severe handling problems in livestock. Moreover, an inherited form of TSE in humans, fatal familial insomnia (FFI), is also associated with sleep abnormalities (Petersen et al., 1992). The disease leads to a gradual reduction in physiological sleep until a complete loss of sleep occurs. Impaired autonomic and motor functions are also manifest (Portaluppi et al., 1994).

The present invention utilizes standard techniques of the art to construct a novel configuration of the PrP gene in cattle that provides resistance to the infective agent analogous to the knock-out construct proposed by Weissmann et al., but at the same time leaves an intact, functional copy of the PrP to perform the normal but unknown role of the PrP gene in tissues where it is expressed. In contrast, the transgenic mice described by Weissmann, et al. do not express a functional copy of the PrP gene.

An alternative to the foregoing homologous recombination method is to augment homologous recombination by either co-transforming fetal fibroblasts with a vector carrying the bacterial RecA protein or the bovine Rad51 protein, or directly binding the corresponding RecA or Rad51 proteins to the single stranded DNA targeting construct prior to transfection. Such procedures are described in, for example, U.S. Provisional Patent Application Ser. No. 60/284,635, filed Apr. 18, 2001, the entire disclosure of which is specifically incorporated herein by reference.

### Example 5

### Generation of a BSE-Resistant Form of the Bovine PrP Gene

Primers were developed to exon 3 of the bovine PrP gene based on published data. One primer pair was used to screen a bovine BAC library, yielding three bacterial clones with overlapping restriction digest profiles.

In order to identify the region of each BAC clone that contained the PrP gene, DNA from each clone was digested with several restriction enzymes including Avril and EcoRI, the DNA transferred to nylon, and the Southern blot probed with radiolabelled PrP fragment from Exon 3.

Restriction fragments that were shown to contain the PrP gene by Southern analysis were then subcloned into plasmid vector pBluescript (pBS), and the ends of the insert were sequenced to verify that all three clones contained bovine PrP.

Since gene targeting requires the use of “isogenic” DNA, or DNA in the targeting vector that is genetically identical to the targeted locus, primers were developed from the sequenced ends of one of the clones and used to amplify the PrP gene from DNA extracted from a bovine fetal fibroblast primary culture by long-range PCR (LR-PCR).

The LR-PCR product was cloned into vector pCR2. 1 and the insert sequenced to verify that the PrP gene had been faithfully amplified. The insert was 100% identical to published sequence data for Exon 3.

In order to convert the wild type triplet sequence (CAG) coding for glutamine (Gln) at amino acid 179 to arginine (Arg) coded to the triplet CGG, a mutagenesis primer was synthesized that would convert the middle base of CAG from A to G thereby producing the required CGG triplet coding for Arg rather than Gln (See FIG. 1).

The mutagenesis protocol of Deng and Nicholoff was used to generate the amino acid change indicated above. Briefly, the vector containing wild-type PrP was denatured and then hybridized with the mutagenesis primer that encodes the desired modification (C to G) and a selection primer that alters a unique restriction site. A second strand is synthesized using DNA polymerase and gaps sealed with DNA ligase. Following transformation into an appropriate host, clones containing the mutagenic plasmid are selected by digestion of isolated DNA with the restriction enzyme that digests the altered site.

To verify that the A to G change has been incorporated at the triplet at position 179, the region flanking the site is sequenced on both strands. As FIG. 1 illustrates, the mutation has been incorporated into bovine PrP. To ensure that no other unintended mutations have been incorporated into the coding region of PrP, both strands were sequenced covering the entire opening reading frame of PrP. No other alterations were detected.

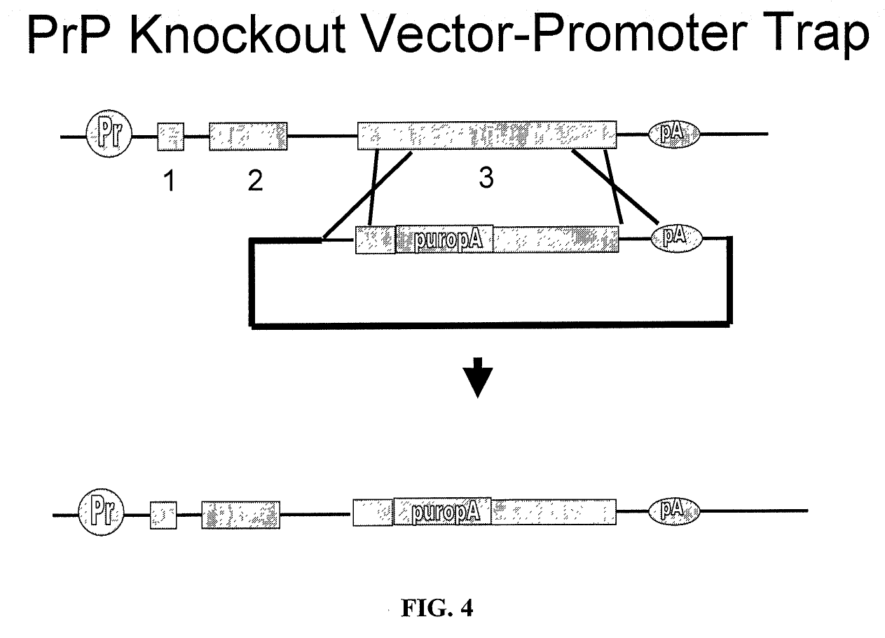

A restriction fragment containing the altered sequence was then subcloned into a transforming vector containing 7.0 kb of isogenic bovine PrP gene, replacing the wild-type sequence with the altered sequence. The coding region is interrupted by a selectable marker, puromycin, that will permit selection of the construct following electroporation into bovine fetal fibroblasts (See FIG. 4).

### Example 6

### Approach

A. Production of PrP-Resistant Animals with Functional Copies of the PrP Gene.

Two transgenes are generated that overexpress a functional copy of the PrP gene and are resistant to conversion to the pathogenic PrP^{Sc} conformation. As there is very high level of amino acid sequence homology in the regions flanking the residue in which the three amino acid substitutions will be made (Prusiner et al., 1993), the substitution is unlikely to disturb secondary and tertiary structure of bovine or cervid PrP. Moreover, the Q171R and E222K are naturally occurring polymorphisms in otherwise perfectly healthy sheep and humans, respectively (Westaway et al., 1994; Shibuya et al., 1998). In addition, when these substitutions were introduced into mouse PrP on plasmids transfected into chronically PrP^{Sc}-infected mouse neuroblastoma cultured cells that readily convert the susceptible mouse allele to PrP^{Sc}, these substitutions prevented such conversion (Zulianello et al., 2000) thus acting in a dominant negative fashion.

B. Construction of the Transgenes:

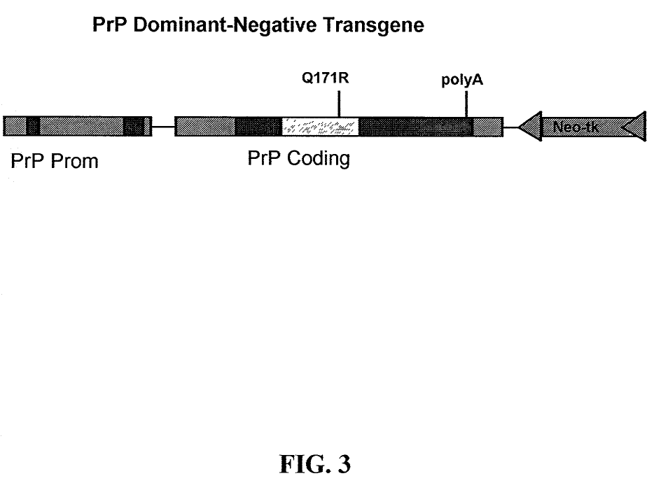

Q171R and Q222K substitutions are introduced by single base modification of a CAG codon coding for Gln to CGG (Arg) or AAG (Lys) by site-directed mutagenesis. FIG. 1 demonstrates that the CAG to CGG modification at amino acid 171 was successfully introduced into bovine PrP using sequencing analysis. The substituted PrP exon 3 sequence including endogenous polyA addition site was then ligated downstream of required bovine PrP promoter elements including exon 1, intron 1 and exon 2 (Inoue et al., 1997) in a SuperCos cosmid vector. A positive-negative selection cassette containing the neomycin resistance gene (+) and the HSV thymidine kinase gene (−) is flanked by loxP sites. FIG. 3 shows the order of these components in the transgene. The cloned DNA is transduced into fetal fibroblasts, neomycin-resistant colonies isolated and expression levels of the transgene determined by Northern and Western analysis. Clones that show high level expression of the transgene are used as donors for nuclear transfer. Calves will be tested for the presence of the transgene by PCR and Southern analysis. The neomycin selectable marker flanked by loxP sites allows Cre-mediated removal of selectable markers after identification of transgenic colonies (Nagy, 2000). This ensures that any animal generated will not be expressing any antibiotic resistance markers.

C. Generation of Homozygous Knockout Animals

The bovine PrP gene was isolated from a bacterial artificial chromosome (BAC) large insert library by PCR screening of pooled clones (Cai et al., 1995). The PrP gene was amplified by long-range PCR to develop isogenic targeting constructs (Barnes, 1994; Randolph et al., 1996) and the gene was disrupted by insertion of a promoterless puromycin-resistance gene into the open reading frame of exon 3. The PrP gene is expressed at high levels in fetal fibroblasts allowing for the very effective promoter trap gene targeting approach (Hasty et al., 1999). Successful targeting of the PrP locus was achieved using a combination of conventional enrichment schemes (promoter trap, isogenic DNA, extensive homology). The targeting scheme used to create the PrP knockout is illustrated in FIG. 4.

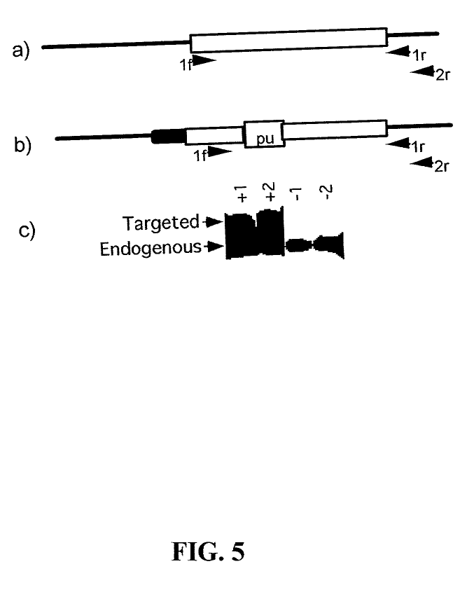

Targeted cells were identified by long range PCR. Long-range PCR is carried out using primers that amplify both targeted and endogenous genes. Two independent sets of PCR primers are used for amplification. PCR products are transferred to nylon by the Southern procedure and hybridized with ³²P-labelled probe to the test loci and puromycin probes. Only DNA displaying a band characteristic of a targeted gene for all PCR products is scored as positive (targeted) (see FIG. 5).

Targeted cells were then used as nuclear donors for somatic cell nuclear transfer. Reconstructed embryos are currently being transferred to recipient cows. To complete the inactivation of the remaining PrP allele, cell are collected from 50 days PrP+/− fetuses generated as described above and are utilized in an analogous targeting procedure, but using hygromycin as the selectable marker (see, e.g., Brown et al., 1997). In both cases the selectable markers are flanked by loxP sites allowing for the removal of the selectable markers once the cells carrying a fully deleted PrP is identified. Using the culture system described by Vasquez et al. (1998), sufficient cell divisions were obtained prior to senescence to undergo two rounds of selection prior to cloning by nuclear transfer. This indicates that it will be possible to remove the selectable markers prior to cloning without the need for an addition round of fetal fibroblast collection.

D. Generating Cervid Transgenes.

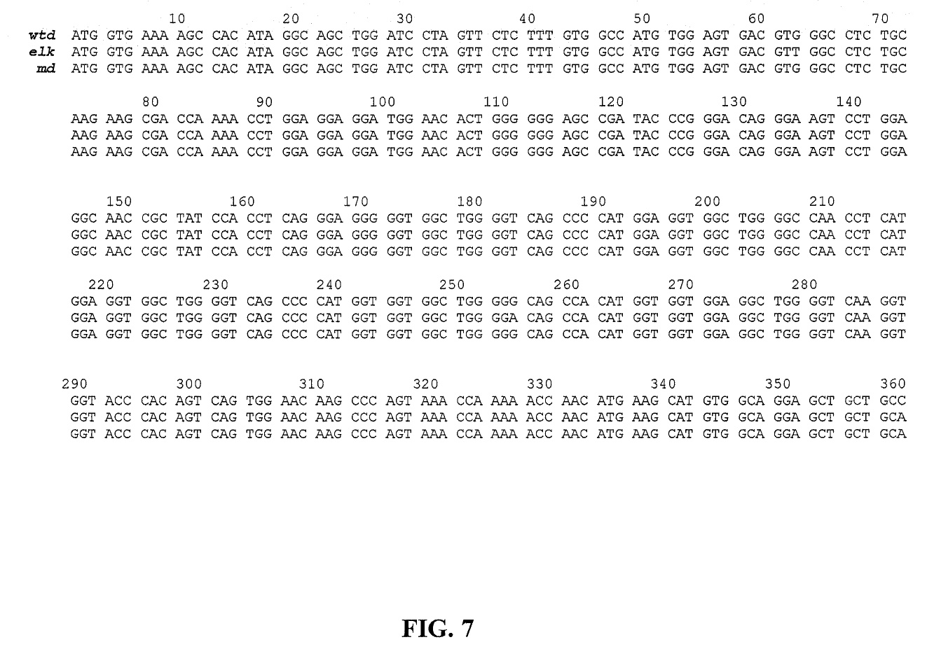

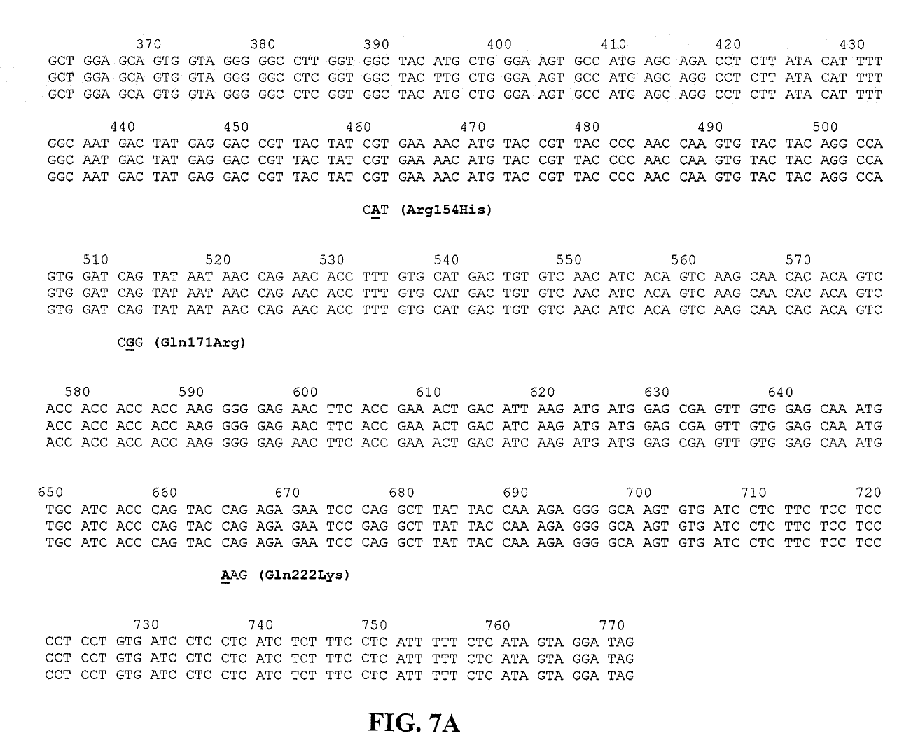

Using the published genomic sequence of sheep and bovine PrP genes (Genbank Accession numbers U67922 (SEQ ID NO:8) and AJ298878 (SEQ ID NO:9), respectively), consensus primers were designed to amplify cervid (deer and elk) genomic DNA by long-range PCR from cervid DNA collected from cultured fibroblasts from each species. Since the DNA from the coding region of white-tail deer (Odocoileus virginianus), mule deer (Odocoileus hemionus) and Rocky Mountain elk (Cervus elaphus nelsoni) have been published (Genbank Accession numbers AF156184 (SEQ ID NO:10), AF009181 (SEQ ID NO:11) and AF156182 (SEQ ID NO:12), respectively), this information is used to create, for example, R154H, Q171R and Q222K alleles by single base alterations in each genomic DNA by site-directed mutagenesis using the same technique used to alter bovine PrP DNA (see FIG. 7). Similarly, consensus primers are used to amplify cervid promoter sequence. Transgenes are introduced into cervid fibroblasts and transgenic animals expressing high levels of each transgene are produced by somatic cell nuclear transfer.

E. Generating Homozygous Knockout Cervids.

Targeting constructs similar to those used to target the bovine PrP gene are constructed using cloned cervid PrP DNA already isolated for the production of cervid transgenes. The methods used to target the cervid PrP locus are identical to that used in bovine targeting. Again, as with bovine, knockout cell lines are used for transfection of appropriate transgenes to generate fully resistant animals expressing the transgene in a null PrP genetic background.

F. Production of Nuclear Transfer Embryos:

For nuclear transplantation bovine acolytes are matured in vitro as described in Hill et al., (2000). Oocytes are removed from medium and placed for 15 minutes in HEPES buffered SOF with 4 mg/ml BSA that contains 7.5 μg/ml cytochalasin B and 5 μg/ml Hoechst 33342. Oocytes will be enucleated using micromanipulation. Only those in which removal of both the polar body and metaphase chromosomes is confirmed, by observation under UV light, will be utilized. Fibroblasts will be prepared by trypsinization of cells at 60-80% confluence and combined with enucleated oocytes using a 30 μm outside diameter glass pipette. Doublets will then be placed into TCM199+10% FCS. The oocyte-fibroblast couplets will be manually aligned and fused in a 3.2 mm fusion chamber that contains Zimmermans cell fusion medium using 2×20 μsec, 1.6 KV/cm DC fusion pulses delivered by a BTX Electrocell Manipulator 200 (BTX Inc. San Diego, Calif.). Oocyte activation will be performed 3-5 hours after fusion, by a 4 minute incubation in 5 μM ionomycin followed by 4 minutes in 3% BSA in H-SOF then 4 minutes in H-SOF. Fusion will be assessed at this time by light microscopy prior to transfer into 100 μM Butyrolactone (Motlik et al., 1998) in SOF for 4 hours. NT embryos will then be cultured in cSOFMaa for 7 days. Embryos will be transferred to synchronized recipients, and pregnancy closely monitored by ultrasonography starting at day 30. These techniques are routinely utilized by the inventors to produce NT blastocysts, cloned fetuses and live cloned calves.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.

U.S. Pat. No. 5,698,763

U.S. Pat. No. 5,789,655

U.S. Pat. No. 5,834,593

U.S. Pat. No. 5,792,901

U.S. Pat. No. 5,834,593

U.S. Pat. No. 5,527,695

U.S. Pat. No. 5,658,772

U.S. Pat. No. 5,440,013

U.S. Pat. No. 5,618,914

U.S. Pat. No. 5,670,155

U.S. Pat. No. 5,446,128

U.S. Pat. No. 5,710,245

U.S. Pat. No. 5,840,833

U.S. Pat. No. 5,859,184

U.S. Pat. No. 5,929,237

U.S. Pat. No. 5,475,085

U.S. Pat. No. 5,672,681

U.S. Pat. No. 5,674,976

U.S. Pat. No. 4,554,101

Araki et al., “Site-specific recombinase, R, encoded by yeast plasmid pSR1,” J. Mol. Biol. 225:25-37, 1992.

Belay, E. D., Gambetti, P., Schonberger, L. B., Parchi, P., Lyon, D. R., Capellari, S., McQuiston, J. H., Bradley, K., Dowdle, G., Crutcher, J. M. and Nichols, C. R. (2001). Creutzfeldt-Jacob disease in unusually young patients who consumed venison. Arch. Neurol. 58, 1673-167 .

Belt, P. B. G. M., Muileman, I. H., Schreuder, B. E. C., Bos-de Ruijter, J., Gielkens, A. L. J., Smits, M. A. (1995). Identification of five allelic variants of the sheep PrP gene and their association with natural scrapie. J. Gen. Virol. 76: 509-517.

Brown JP, Wei W, Sedivy J M. Bypass of senescence after disruption of p21CIP1/WAF1 gene in normal diploid human fibroblasts. Science 1997, 277: 831-834.

Bueler, H, Aguzzi, A., Sailer, A., Greiner, R. A., Autereid, P., Aguet, M. and C. Weissmann. (1993) Mice devoid of PrP are resistant to Scrapie. Cell 73: 1339-1347.

Cai, L., Taylor, J. F., Wing R. A., Gallagher, D. S., Woo, S. S., Davis, S. K.(1995) Construction and characterization of a bovine bacterial artificial chromosome library. Genomics. 29: 413-25.

Clouscard et a., J. Gen. Virol. (76)2097-2101, 1995.

Enomoto et al., “Mapping of the pin locus coding for a site-specific recombinase that causes flagellar-phase variation in Escherichia coli K-12,” J. Bacteriol., 156:663-668, 1983.

Foster J. Goldmann W. Parnham D. Chong A. Hunter N. (2001). Partial dissociation of PrP(Sc) deposition and vacuolation in the brains of scrapie and BSE experimentally affected goats. Journal of General Virology. 82::267-73.

Gabizon, R.; Rosenmann, H.; Meiner, Z.; Kahana, I.; Kahana, E.; Shugart, Y.; Ott, J.; and Prusiner, S. B., Am. J. Hum. Genet., 53, 828-835, 1993.

Goldmann, W., Hunter, N., Martin, T., Dawson, M., Hope, J. (1991). Different forms of the bovine PrP gene have five or six copies of a short, G-C-rich element withinthe protein-coding exon. J. Gen. Virol. 72: 201-204.

Goldmann, W., Hunter, N., Smith, G., Foster, J., Hope, J. (1994). PrP genotype and agent effects in scrapie: change in allelic interaction with different agents in sheep, a natural host of scrapie. J. Gen. Virol. 75: 989-995.

Golic and Lindquist, “The FLP recombinase of yeast catalyses site-specific recombination in the Drosophila genome,” Cell, 59:499-509, 1989.

Hasty P, Abuin A, Bradley A. (1999) Gene targeting, principles, and practice in mammalian cells. In Gene Targeting, 2^{nd} Edition. Edited by A. L. Joyner. Oxford U. P., pp 1-35.

Hill J R, Winger Q A, Long C R, Looney C R, Thompson J A, Westhusin M E. Development rates of male bovine nuclear transfer embryos derived from adult and fetal cells. Biology of Reproduction 2000; 62:1135-1140.

Hunter et al., Vet. Rec., (135):400-403, 1994.

Hunter, N., Goldmann, W., Smith, G., Hope, J. (1994). Frequencies of PrP gene variants in healthy cattle and cattle with BSE in Scotland. Vet. Rec. 135: 400-403.

Inoue, S., Tanaka, M., Horiuchi, M., Ishiguro, N., Shinagawa, M. (1997). Characterization of the bovine prion protein gene: the expression requires interaction between the promoter and intron. J. Vet. Med. Sci. 59: 175-183.

Johannesson et al., 1999, “Bicyclic tripeptide mimetics with reverse turn inducing properties.” J. Med. Chem. 42:601-608.

LaPlanche, J. L., Chatelain, J., Westaway, D., Thomas, S., Dussaucy, M., Brugere-Picoux, J., Launey, J. M. (1993). PrP polymorphisms associated with natural scrapie discovered by denaturing gradient gel electrophoresis. Genomics 15: 30-37.

Maeser and Kahmann, “The GIN recombinase of phage Mu can catalyse site-specific recombination in plant protoplasts,” Mol. Gen. Genet., 230:170-176, 1991.

Motlik J. Pavlok A. Kubelka M. Kalous J. Kalab P. Interplay between CDC2 kinase and MAP kinase pathway during maturation of mammalian oocytes. Theriogenology. 49(2):461-9, Jan 15, 1998

Nagy A. Cre-recombinase: the universal reagent for genome tailoring. Genesis 2000, 26: 99-109.

Peretz, D.; Williamson, R. A.; Matsunaga, Y.; Serban, H.; Pinilla, C.; Bastidas, R.; Rozenshteyn, R.; James, T. L.; Houghten, R. A.; Cohen, F. E., et al.; J. Mol. Biol., 173, 614-622, 1997.

Piedrahita J A, Wells D N, Miller A L, Oliver J E, Berg M C , Peterson A J, Tervit R H. 2001. Successful cloning in cattle with cytoplasts obtained from follicles of 1-3 mm in diameter. Mol. Reprod. Dev. In Press.

Prusiner, S. B. Proc Natl Acad Sci USA (23) 13363-83 (1995)

Prusiner, S. B., Fuzi, M., Scott, M., Serban, D., Taraboulos, A., Gabriel, J -M., Wells, G. A. H., Wilesmith, J. W. Bradley, R., DeArmond, S. J., Kristensson, K. (1993). Immunologic and molecular biologic studies of prion proteins in bovine spongiform encephalopathy. J. Infectious Diseases. 167: 602-613.

Raymond, G. J., Bossers, A., Raymond, L. D., o'Rourke, K. I., McHolland, L. E., Bryant, P. K., Miller, M. W., Williams, E. S., Smits, M. and Caughy, B.(2000). Evidence of a molecular barrier limiting susceptibility of humans, cattle and sheep to chronic wasting disease. EMBO J. 19: 4425-4430.

Ryan and Womack, Animal Genet., (24):23-26, 1993.

Sauer, “Functional expression of the cre-lox site-specific recombination system in the yeast Saccharomyces cerevisiae,” Mol. and Cell. Biol., 7: 2087-2096, 1987.

Shibuya, S., Higuchi, J., Shin, R. -W., Tateishi, J., Kitamoto, T. (1998). Protective prion protein polymorphisms against sporadic Creutzfeldt-Jakob disease. The Lancet 352: 419.

Tobler, I. Gaus, S. E., DeBoer, T., Achermann, P., Fischer, M., Rulicke, T., Moser, M., Oesch, B., McBride, P. A. and J. C. Manson. (1996) Altered circadian activity rhythms and sleep in mice devoid of prion protein. Nature 380:639-642.

Vasquez, J. C., Nogues, C., Rucker, E. B., Piedrahita, J. A (1998). Factors affecting the efficiency of introducing precise genetic changes in ES cells by homologous recombination: tag-and-exchange versus the cre-loxP system. Transgenic Research 7: 181-193.

Vita et al., (1998), “Novel miniproteins engineered by the transfer of active sites to small natural scaffolds. 47(1):93-100.

Weisshoff et al., (1999), “Mimicry of beta II’-turns of proteins in cyclic pentapeptides with one and without D-amino acids. Eur J Biochem. 259(3):776-88

Westaway, D., Zuliani, V., Cooper, C. M., Da Costa, M., Neuman, S., Jenny, A. L., Detwiler, L., Prusiner, S. B. (1994). Homozygosity for prion protein alleles encoding glutamine 171 renders sheep susceptible to natural scrapie. Genes Devel. 8: 959-969.

Westhusin M, Burghardt R C, Ruglia T N, Willingham L A, Liu L, Shin T, Howe L M, Kraemer D C. Potential for cloning dogs. J. Reprod. Fert 2000; In press.

Zulianello, L., Kanecko, K., Scott, M., Erpel, S., Han, D., Cohen, F. E., Prusiner, S. B. (2000). Dominant-negative inhibition of prion formation diminished by deletion mutagenesis of the prion protein. J. Virol. 74: 4351-4360.

What is claimed is:

1. A transgenic bovine comprising a transgene encoding a mutant PrP polypeptide comprising the polypeptide sequence of SEQ ID NO:2 in which an amino acid substitution has been made at position 171 of said sequence that renders said bovine resistant to bovine spongiform encephalopathy disease.

2. The transgenic bovine of claim 1, wherein the mutant PrP polypeptide further comprises an amino acid substitution at a position of said sequence selected from the group consisting of 154 and 222.

3. The transgenic bovine of claim 2, wherein the amino acid substitution comprises substitution with an amino acid selected from the group consisting of histidine, lysine or arginine.

4. The transgenic bovine of claim 1, wherein the glutamine residue at said position 171 has been substituted with histidine, lysine or arginine.

5. The transgenic bovine of claim 4, wherein the glutamine residue at said position 171 has been substituted with arginine.

6. The transgenic bovine of claim 1, further defined as produced by a method comprising introducing a transgene encoding said mutant PrP polypeptide into the genome of a bovine embryo and allowing the embryo to develop into a bovine whose somatic and germ cells comprise said transgene.

7. A progeny of any generation of the transgenic bovine of claim 6, wherein said progeny comprises said transgene.

8. A fertilized embryo of the transgenic bovine of claim 1, wherein said embryo comprises said transgene.

9. The transgenic bovine of claim 1, further defined as lacking a functional wild type PrP gene.

10. The transgenic bovine of claim 9, wherein said wild type PrP gene has been replaced with a null allele by homologous recombination.

11. A method of producing a transgenic bovine resistant to BSE comprising:
a) introducing into a bovine embryo a transgene encoding a mutant PrP polypeptide comprising the polypeptide sequence of SEQ ID NO:2 in which an amino acid substitution has been made at position 171 of said sequence; and
b) allowing the embryo to develop into a bovine the somatic and germ cells of which express said transgene, thereby rendering the transgenic bovine resistant to BSE.

12. The method of claim 11, wherein the mutant PrP polypeptide further comprises an amino acid substitution at a position of said sequence selected from the group consisting of 154 and 222.

13. The transgenic bovine of claim 12, wherein the amino acid substitution comprises substitution with an amino acid selected from the group consisting of histidine, lysine or arginine.

14. The method of claim 11, wherein the glutamine residue at said position 171 has been substituted with histidine, lysine or arginine.

15. The method of claim 14, wherein the glutamine residue at said position 171 has been substituted with arginine.

16. The method of claim 11, wherein the transgenic bovine is further defined as lacking a functional wild type PrP gene.

17. The method of claim 16, wherein said wild type PrP gene has been replaced with a null allele by homologous recombination.

18. A transgenic cervid comprising a transgene encoding a mutant PrP polypeptide comprising the polypeptide sequence of SEQ ID NO:2 in which an amino acid substitution has been made at position 171 of said sequence that renders said cervid resistant to transmissible spongiform encephalopathy disease.

19. The transgenic cervid of claim 18, wherein the mutant PrP polypeptide further comprises an amino acid substitution at a position of said sequence selected from the group consisting of 154 and 222.

20. The transgenic cervid of claim 19, wherein the amino acid substitution comprises substitution with an amino acid selected from the group consisting of histidine, lysine or arginine.

21. The transgenic cervid of claim 18, wherein the glutamine residue at said position 171 has been substituted with histidine, lysine or arginine.

22. The transgenic cervid of claim 21, wherein the glutamine residue at said position 171 has been substituted with arginine.

23. The transgenic cervid of claim 18, further defined as produced by a method comprising introducing a transgene encoding said mutant PrP polypeptide into the genome of a cervid embryo and allowing the embryo to develop into a cervid whose somatic and germ cells comprise said transgene.

24. A progeny of any generation of the transgenic cervid of claim 23, wherein said progeny comprises said transgene.

25. A fertilized embryo of the transgenic cervid of claim 18, wherein said embryo comprises said transgene.

26. The transgenic cervid of claim 18, further defined as lacking a functional wild type PrP gene.

27. The transgenic cervid of claim 26, wherein said wild type PrP gene has been replaced with a null allele by homologous recombination.

28. A method of producing a transgenic cervid resistant to transmissible spongiform encephalopathy comprising:
a) introducing into a cervid embryo a transgene encoding a mutant PrP polypeptide comprising the polypeptide sequence of SEQ ID NO:2 in which an amino acid substitution has been made at position 171 of said sequence; and
b) allowing the embryo to develop into a cervid the somatic and germ cells of which express said transgene, thereby rendering the transgenic cervid resistant to transmissible spongiform encephalopathy.

29. The transgenic cervid of claim 28, wherein the mutant PrP polypeptide further comprises an amino acid substitution at a position of said sequence selected from the group consisting of 154 and 222.

30. The transgenic cervid of claim 29, wherein the amino acid substitution comprises substitution with an amino acid selected from the group consisting of histidine, lysine or arginine.

31. The transgenic cervid of claim 28, wherein the glutamine residue at said position 171 has been substituted with histidine, lysine or arginine.

32. The transgenic cervid of claim 31, wherein the glutamine residue at said position 171 has been substituted with arginine.

33. The method of claim 28, wherein the transgenic cervid is further defined as lacking a functional wild type PrP gene.

34. The method of claim 33, wherein said wild type PrP gene has been replaced with a null allele by homologous recombination.
